SEQUENCE LISTING (2 sequences)

SEQ ID NO: 1 - DNA, 584 bp, synthetic construct
  source 1..584: mol_type = other DNA
ccactacgggtctaggctgcccatgtaaggaggcaaggcctggggacacccgagatgcctggttataattaacccagacatgtggctgcccccccccccccaacacctgctgcctgagcctcacccccaccccggtgcctgggtcttaggctctgtacaccatggaggagaagctcgctctaaaaataaccctgtccctggtggatcccctgcatgcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 2 - protein, 952 aa, synthetic construct
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

(19) United States
(12) Patent Application Publication
(10) Pub. No.: US 2025/0135038 A1
(43) Pub. Date: May 1, 2025

(54) COMPOSITIONS AND METHODS FOR IMPROVED TREATMENT OF POMPE DISEASE

(71) Applicant: Astellas Gene Therapies, Inc., South San Francisco, CA (US)

(72) Inventors: Tahseen MOZAFFAR, Orange, CA (US); Angela SMITH, San Francisco, CA (US)

(21) Appl. No.: 18/835,480

(22) PCT Filed: Feb. 3, 2023
(86) PCT No.: PCT/US2023/061951

§ 371 Date: Aug. 2, 2024

Related U.S. Application Data
(60) Provisional application No. 63/402,850, filed on Aug. 31, 2022.
(60) Provisional application No. 63/306,361, filed on Feb. 3, 2022.

(51) Int. Cl. A61K 48/00; A61K 31/573; A61K 38/47; A61P 3/08; C12N 15/86

(52) U.S. Cl. CPC A61K 48/0058; A61K 31/573; A61K 38/47; A61P 3/08; C12N 15/86; C12Y 302/0102; C12N 2750/14143

(57) ABSTRACT

The present invention provides methods for treating co-morbid transaminasemia associated with a glycogen storage disorder. In certain embodiments, the invention provides methods for assessing readiness of a subject with Pompe disease for combination therapy with an anti-transaminitis agent.

### SEQUENCE LISTING

The present application is being filed along with a Sequence Listing in electronic format. The Sequence Listing is provided as a file entitled 51037-066WO3_Sequence_Listing_2_2_23.xml created on Feb. 2, 2023, which is 4.1 bytes in size. The information in electronic format of the sequence listing is incorporated herein by reference in its entirety.

### FIELD OF THE INVENTION

The present disclosure relates to the field of gene therapy and provides compositions and methods for ameliorating genetic disorders.

### BACKGROUND OF THE INVENTION

Pompe disease is a lysosomal storage disorder caused by mutations in the acid alpha-glucosidase (GAA) gene, which encodes an enzyme responsible for processing lysosomal glycogen. Patients with Pompe disease exhibit clinical phenotypes across a variety of tissues, including glycogen buildup in cells, deficits in cardiac, respiratory, and skeletal muscle function, and central nervous system pathology. Some of these deficits are significantly ameliorated by enzyme replacement therapy (ERT) using recombinant human GAA (rhGAA). Clinical efficacy has been limited by the immunogenicity of hGAA ERT and the lack of uptake of rhGAA into some affected tissues. Recently, gene therapy approaches involving the delivery of GAA have been developed for the treatment of Pompe disease. However, there is a need in the art for improved methods of administering gene therapy to patients having Pompe disease.

### SUMMARY OF THE INVENTION

The present disclosure provides compositions and methods that can be used for treating glycogen storage disorders, such as type II glycogen storage disorder, which is also referred to herein as Pompe disease. Using the compositions and methods of the disclosure, a patient (e.g., a mammalian patient, such as a human patient) having Pompe disease may be administered a viral vector, such as an adeno-associated viral (AAV) vector, that contains a transgene encoding acid alpha-glucosidase (GAA). The AAV vector may be, for example, a pseudotyped AAV vector, such as an AAV vector containing AAV2 inverted terminal repeats packaged within capsid proteins from AAV8 (AAV2/8) or AAV9 (AAV2/9). The transgene may, for example, be operably linked to a transcription regulatory element, such as a promoter that induces gene expression in a muscle cell and/or a neuronal cell. Exemplary promoters that may be used in conjunction with the compositions and methods of the disclosure are a muscle creatine kinase (MCK) promoter and cytomegalovirus (CMV) promoter, among others. The AAV vector may be administered to the patient in a therapeutically effective amount, such as in an amount of from 1×10¹³ vector genomes (vg) per kg of body weight of the subject (vg/kg) to 3×10¹⁴ vg/kg (e.g., in an amount of from 3×10¹³ vg/kg to 2×10¹⁴ vg/kg, 4×10¹³ vg/kg to 1×10¹⁴ vg/kg, 4×10¹³ vg/kg, 5×10¹³ vg/kg, 6×10¹³ vg/kg, 7×10¹³ vg/kg, 8×10¹³ vg/kg, 9×10¹³ vg/kg, or 1×10¹⁴ vg/kg).

In one aspect, the disclosure provides a method of treating Pompe disease in a human patient in need thereof, the method including administering to the patient (i) a therapeutically effective amount of a viral vector including a transgene encoding GAA and (ii) an anti-transaminitis agent.

In another aspect, the disclosure provides a method of reducing glycogen accumulation in muscle tissue and/or in neuronal tissue in a human patient diagnosed as having Pompe disease, the method including administering to the patient (i) a therapeutically effective amount of a viral vector including a transgene encoding GAA and (ii) an anti-transaminitis agent.

In another aspect, the disclosure provides a method of improving pulmonary function in a human patient diagnosed as having Pompe disease (e.g., LOPD), the method including administering to the patient (i) a therapeutically effective amount of a viral vector including a transgene encoding GAA and (ii) an anti-transaminitis agent.

In another aspect, the disclosure provides a method of increasing GAA expression in a human patient diagnosed as having Pompe disease (e.g., LOPD), the method including administering to the patient (i) a therapeutically effective amount of a viral vector including a transgene encoding GAA and (ii) an anti-transaminitis agent.

In some embodiments of the foregoing aspects, the anti-transaminitis agent is administered to the patient in one or more doses that commence within 48 weeks of administration of the viral vector to the patient (e.g., 36 weeks, 24 weeks, 12 weeks, 10 weeks, 8 weeks, 6 weeks, or 4 weeks) of administration of the viral vector to the patient. In some embodiments of the foregoing aspects, the anti-transaminitis agent is administered to the patient in one or more doses that commence within 12 weeks of administration of the viral vector to the patient (e.g., 10 weeks, 8 weeks, 6 weeks, or 4 weeks) of administration of the viral vector to the patient.

In another aspect, the disclosure provides a method of treating Pompe disease in a human patient in need thereof and who has been previously administered an anti-transaminitis agent, the method including administering to the patient a therapeutically effective amount of a viral vector including a transgene encoding GAA.

In another aspect, the disclosure provides a method of reducing glycogen accumulation in muscle tissue and/or in neuronal tissue in a human patient diagnosed as having Pompe disease and who has been previously administered an anti-transaminitis agent, the method including administering to the patient a therapeutically effective amount of a viral vector including a transgene encoding GAA.

In another aspect, the disclosure provides a method of improving pulmonary function in a human patient diagnosed as having Pompe disease and who has been previously administered an anti-transaminitis agent, the method including administering to the patient a therapeutically effective amount of a viral vector including a transgene encoding GAA.

In another aspect, the disclosure provides a method of increasing GAA expression in a human patient diagnosed as having Pompe disease and who has been previously administered an anti-transaminitis agent, the method including administering to the patient a therapeutically effective amount of a viral vector including a transgene encoding GAA.

In another aspect, the disclosure provides a method of treating Pompe disease in a human patient in need thereof, the method including: (a) administering to the patient a viral vector including a transgene encoding GAA, (b) monitoring the patient for development of transaminasemia, hyperbilirubinemia, or one or more symptoms thereof, and, if the patient exhibits transaminasemia, hyperbilirubinemia, or one or more symptoms thereof, (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of reducing glycogen accumulation in muscle tissue and/or in neuronal tissue in a human patient diagnosed as having Pompe disease (e.g., LOPD), the method including: (a) administering to the patient a viral vector including a transgene encoding GAA, (b) monitoring the patient for development of transaminasemia, hyperbilirubinemia, or one or more symptoms thereof, and, if the patient exhibits transaminasemia, hyperbilirubinemia, or one or more symptoms thereof, (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of improving pulmonary function in a human patient diagnosed as having Pompe disease (e.g., LOPD), the method including: (a) administering to the patient a viral vector including a transgene encoding GAA, (b) monitoring the patient for development of transaminasemia, hyperbilirubinemia, or one or more symptoms thereof, and, if the patient exhibits transaminasemia, hyperbilirubinemia, or one or more symptoms thereof, (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of increasing GAA expression in a human patient diagnosed as having Pompe disease (e.g., LOPD), the method including: (a) administering to the patient a viral vector including a transgene encoding GAA, (b) monitoring the patient for development of transaminasemia, hyperbilirubinemia, or one or more symptoms thereof, and, if the patient exhibits transaminasemia, hyperbilirubinemia, or one or more symptoms thereof, (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of treating Pompe disease in a human patient in need thereof, the method including: (a) administering to the patient a viral vector including a transgene encoding GAA, (b) determining that the patient exhibits transaminasemia, hyperbilirubinemia, or one or more symptoms thereof, and (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of reducing glycogen accumulation in muscle tissue and/or in neuronal tissue in a human patient diagnosed as having Pompe disease (e.g., LOPD), the method including: (a) administering to the patient a viral vector including a transgene encoding GAA, (b) determining that the patient exhibits transaminasemia, hyperbilirubinemia, or one or more symptoms thereof, and (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of improving pulmonary function in a human patient diagnosed as having Pompe disease (e.g., LOPD), the method including: (a) administering to the patient a viral vector including a transgene encoding GAA, (b) determining that the patient exhibits transaminasemia, hyperbilirubinemia, or one or more symptoms thereof, and (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of increasing GAA expression in a human patient diagnosed as having Pompe disease (e.g., LOPD), the method including: (a) administering to the patient a viral vector including a transgene encoding GAA, (b) determining that the patient exhibits transaminasemia, hyperbilirubinemia, or one or more symptoms thereof, and (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of treating Pompe disease in a human patient in need thereof that is one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older), the method including: (a) administering to the patient a therapeutically effective amount of a viral vector including a transgene encoding GAA, (b) monitoring the patient for development of transaminasemia, hyperbilirubinemia, or one or more symptoms thereof, and, if the patient exhibits transaminasemia, hyperbilirubinemia, or one or more symptoms thereof, (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of reducing glycogen accumulation in muscle tissue and/or in neuronal tissue in a human patient diagnosed as having Pompe disease (e.g., LOPD), the method including: (a) administering to the patient a therapeutically effective amount of a viral vector including a transgene encoding GAA, (b) monitoring the patient for development of transaminasemia, hyperbilirubinemia, or one or more symptoms thereof, and, if the patient exhibits transaminasemia, hyperbilirubinemia, or one or more symptoms thereof, (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of improving pulmonary function in a human patient diagnosed as having Pompe disease (e.g., LOPD), the method including: (a) administering to the patient a therapeutically effective amount of a viral vector including a transgene encoding GAA, (b) monitoring the patient for development of transaminasemia, hyperbilirubinemia, or one or more symptoms thereof, and, if the patient exhibits transaminasemia, hyperbilirubinemia, or one or more symptoms thereof, (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of increasing GAA expression in a human patient diagnosed as having Pompe disease (e.g., LOPD), the method including: (a) administering to the patient a therapeutically effective amount of a viral vector including a transgene encoding GAA, (b) monitoring the patient for development of transaminasemia, hyperbilirubinemia, or one or more symptoms thereof, and, if the patient exhibits transaminasemia, hyperbilirubinemia, or one or more symptoms thereof, (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of treating Pompe disease in a human patient in need thereof that is one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older), the method including: (a) administering to the patient a therapeutically effective amount of a viral vector including a transgene encoding GAA, (b) determining that the patient exhibits transaminasemia, hyperbilirubinemia, or one or more symptoms thereof, and (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of reducing glycogen accumulation in muscle tissue and/or in neuronal tissue in a human patient diagnosed as having Pompe disease that is one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older), the method including: (a) administering to the patient a therapeutically effective amount of a viral vector including a transgene encoding GAA, (b) determining that the patient exhibits transaminasemia, hyperbilirubinemia, or one or more symptoms thereof, and (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of improving pulmonary function in a human patient diagnosed as having Pompe disease that is one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older), the method including: (a) administering to the patient a therapeutically effective amount of a viral vector including a transgene encoding GAA, (b) determining that the patient exhibits transaminasemia, hyperbilirubinemia, or one or more symptoms thereof, and (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of increasing GAA expression in a human patient diagnosed as having Pompe disease that is one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older), the method including: (a) administering to the patient a therapeutically effective amount of a viral vector including a transgene encoding GAA, (b) determining that the patient exhibits transaminasemia, hyperbilirubinemia, or one or more symptoms thereof, and (c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

In another aspect, the disclosure provides a method of treating or preventing transaminasemia or hyperbilirubinemia in a human patient that has Pompe disease and who has been previously administered a viral vector including a transgene encoding GAA in an amount of from 1×10¹³ vg/kg to 3×10¹⁴ vg/kg (e.g., from 1×10¹³ vg/kg to 6×10¹³ vg/kg, from 1×10¹³ vg/kg to 5×10¹³ vg/kg, from 1×10³ vg/kg to 4×10³ vg/kg, from 1×10³ vg/kg to 3×10³ vg/kg, from 2×10³ vg/kg to 6×10¹³ vg/kg, from 2×10¹³ vg/kg to 5×10¹³ vg/kg, or from 2×10¹³ vg/kg to 4×10¹³ vg/kg), the method including administering to the patient an anti-transaminitis agent.

In another aspect, the disclosure provides a method of treating or preventing transaminasemia or hyperbilirubinemia in a human patient that has Pompe disease (e.g., LOPD), has been previously administered a viral vector including a transgene encoding GAA, and that was one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older) at the time of administration of the viral vector, the method including administering to the patient an anti-transaminitis agent. In some embodiments, the patient is 18 years old or older (e.g., 19 years old or older, 20 years old or older, 25 years old or older, 30 years old or older, 40 years old or older, or 50 years old or older) at the time of administration of the viral vector.

In some embodiments of the foregoing aspect, the patient is or was one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older) at the time of administration of the viral vector. In some embodiments, the patient is or was 18 years old or older (e.g., 19 years old or older, 20 years old or older, 25 years old or older, 30 years old or older, 40 years old or older, or 50 years old or older) at the time of administration of the viral vector.

In some embodiments of any of the foregoing aspects, the patient is or was from one year old to 40 years old (e.g., 1 year old to 35 years old, 2 years old to 30 years old, 3 years old to 25 years old, 4 years old to 20 years old, or 18 years old) at the time of administration of the viral vector.

In some embodiments of any of the foregoing aspects, the viral vector is or was administered to the patient in an amount of from 1×10¹³ vg/kg to 3×10¹⁴ vg/kg (e.g., from 1×10¹³ vg/kg to 6×10¹³ vg/kg, from 1×10¹³ vg/kg to 5×10¹³ vg/kg, from 1×10¹³ vg/kg to 4×10¹³ vg/kg, from 1×10¹³ vg/kg to 3×10¹³ vg/kg, from 2×10¹³ vg/kg to 6×10¹³ vg/kg, from 2×10¹³ vg/kg to 5×10¹³ vg/kg, or from 2×10¹³ vg/kg to 4×10¹³ vg/kg). In some embodiments, the viral vector is or was administered to the patient in an amount of from 3×10¹³ vg/kg to 6×10¹³ (e.g., from 4×10¹³ vg/kg to 5×10¹³ vg/kg).

In some embodiments of the foregoing aspects, the viral vector is or was administered to the patient in a single dose including the amount.

In some embodiments of the foregoing aspects, the viral vector is or was administered to the patient in two or more doses that, together, include the amount. In some embodiments, the viral vector is or was administered to the patient in two or more doses that each, individually, include the amount. In some embodiments, the two or more doses are separated from one another by one year or more. In some embodiments, the two or more doses are administered to the patient within 12 months of one another.

In some embodiments of any of the foregoing aspects, the viral vector is selected from the group consisting of AAV, adenovirus, lentivirus, retrovirus, poxvirus, baculovirus, herpes simplex virus, vaccinia virus, and a synthetic virus. In some embodiments, the viral vector is an AAV. In some embodiments, the AAV is an AAV1, AAV2, AAV3, AAV4, AAV5, AAV6, AAV7, AAV8, AAV9, AAVrh10, or AAVrh74 serotype. In some embodiments, the viral vector is a pseudotyped AAV. In some embodiments, the pseudotyped AAV is AAV2/8. In some embodiments, the pseudotyped AAV is AAV2/9.

In some embodiments of any of the foregoing aspects, the transgene encoding GAA is operably linked to a promoter that induces expression of the transgene in a muscle and/or neuronal cell. In some embodiments, the promoter is a muscle MCK promoter, MCK promoter, chicken beta actin promoter, CMV promoter, myosin light chain-2 promoter, alpha actin promoter, troponin 1 promoter, Na⁺/Ca²⁺ exchanger promoter, dystrophin promoter, alpha7 integrin promoter, brain natriuretic peptide promoter, alpha B-crystallin/small heat shock protein promoter, alpha myosin heavy chain promoter, or atrial natriuretic factor promoter.

In some embodiments of any of the foregoing aspects, the GAA is operably linked to an enhancer that induces expression of the transgene in a muscle and/or neuronal cell. In some embodiments, the enhancer is a CMV enhancer, a MEF2 enhancer, or a MyoD enhancer.

In some embodiments of any of the foregoing aspects, the viral vector is or was administered to the patient by way of intravenous, intrathecal, intracisternal, intracerebroventricular, or intramuscular administration to the patient administration.

In some embodiments of any of the foregoing aspects, the anti-transaminitis agent is selected from the group consisting of a corticosteroid, a farnesoid X receptor (FXR) ligand, a fibroblast growth factor 19 (FGF-19) mimetic, a Takeda-G-protein-receptor-5 (TGR5) agonist, a peroxisome proliferator-activated receptor (PPAR) agonist, a PPAR-alpha agonist, a PPAR-delta agonist, a dual PPAR-alpha and PPAR-delta agonist, an apical sodium-dependent corticosteroid transporter (ASBT) inhibitor, an immunomodulatory drug, an antifibrotic therapy, and a nicotinamide adenine dinucleotide phosphate oxidase (NOX) inhibitor. In some embodiments, (i) the corticosteroid is cortisone, prednisone, prednisolone, methylprednisolone, dexamethasone, betamethasone, or hydrocortisone; (ii) the bile acid is ursodeoxycholic acid or nor-ursodeoxycholic acid; (iii) the FXR ligand is obeticholic acid, cilofexor, tropifexor, tretinoin, or EDP-305; (iv) the FGF-19 mimetic is aldafermin; (v) the TGR5 agonist is INT-777 or INT-767; (vi) the PPAR agonist is bezafibrate, seladelpar, or elafibrinor; (vii) the PPAR-alpha agonist is fenofibrate; (viii) the PPAR-delta agonist is seladelpar; (ix) the dual PPAR-alpha and PPAR-delta agonist is elafibranor; (x) the ASBT inhibitor is odevixibat, maralixibat, or linerixibat; (xi) the immunomodulatory drug is rituximab, abatacept, ustekinumab, infliximab, baricitinib, or FFP-104; (xii) the antifibrotic therapy is a vitamin D receptor agonist or simtuzumab; and/or (xiii) the NOX inhibitor is setanaxib. In some embodiments, the corticosteroid is prednisolone.

In some embodiments of the foregoing aspect, the corticosteroid is administered to the patient in a single dose.

In some embodiments of the foregoing aspect, the corticosteroid is administered to the patient in a plurality of doses.

In some embodiments of the foregoing aspect, the corticosteroid is administered to the patient in an amount of from 0.1 mg/kg/dose to 2 mg/kg/dose (e.g., 0.2 mg/kg/dose to 1.9 mg/kg/dose, 0.3 mg/kg/dose to 1.8 mg/kg/dose, 0.4 mg/kg/dose to 1.7 mg/kg/dose, 0.5 mg/kg/dose to 1.6 mg/kg/dose, 1 mg/kg/dose to 1.5 mg/kg/dose). In some embodiments the corticosteroid is administered to the patient in an amount of 0.5 mg/kg/dose. In some embodiments the corticosteroid is administered to the patient in an amount of 1 mg/kg/dose. In some embodiments the corticosteroid is administered to the patient in an amount of 2 mg/kg/dose.

In some embodiments of the foregoing aspect, the corticosteroid is administered to the patient in an amount of from 1 mg to 120 mg (e.g., 2 mg to 119 mg, 3 mg to 118 mg, 4 mg to 117 mg, 5 mg to 116 mg, 10 mg to 115 mg, 20 mg to 110 mg, 30 mg to 100 mg, 40 mg to 90 mg, 50 mg to 80 mg, or 60 mg to 70 mg). In some embodiments, the corticosteroid is administered to the patient in an amount of 30 mg. In some embodiments, the corticosteroid is administered to the patient in an amount of 60 mg. In some embodiments, the corticosteroid is administered to the patient in an amount of 120 mg.

In some embodiments of the foregoing aspect, the corticosteroid is administered to the patient in one or more (e.g., one or more, two or more, three or more, four or more, five or more, six or more, seven or more, eight or more, nine or more, or ten or more) doses per day, week, or month. In some embodiments of the foregoing aspect, the corticosteroid is administered to the patient in one or more (e.g., one or more, two or more, three or more, four or more, five or more, six or more, seven or more, eight or more, nine or more, or ten or more) doses per day, (e.g., one dose per day, in two doses per day, three doses per day, four doses per day, or five doses per day). In some embodiments, the corticosteroid is administered to the patient in one dose per day.

In some embodiments of the foregoing aspect, the corticosteroid is administered to the patient in an amount of from 1 mg/day to 120 mg/day (e.g., 2 mg/day to 119 mg/day, 3 mg/day to 118 mg/day, 4 mg/day to 117 mg/day, 5 mg/day to 116 mg/day, 10 mg/day to 115 mg/day, 20 mg/day to 110 mg/day, 30 mg/day to 100 mg/day, 40 mg/day to 90 mg/day, 50 mg/day to 80 mg/day, or 60 mg/day to 70 mg/day). In some embodiments, the corticosteroid is administered to the patient in an amount of 30 mg/day. In some embodiments, the corticosteroid is administered to the patient in an amount of 60 mg/day. In some embodiments, the corticosteroid is administered to the patient in an amount of 120 mg/day.

In some embodiments of the foregoing aspect, the corticosteroid is administered to the patient by way of a unit dosage form including 5 mg of the corticosteroid.

In some embodiments of the foregoing aspect, the corticosteroid is administered to the patient by way of a unit dosage form including 10 mg of the corticosteroid.

In some embodiments of the foregoing aspect, the corticosteroid is administered to the patient by way of a unit dosage form including 15 mg of the corticosteroid.

In some embodiments of the foregoing aspect, the corticosteroid is administered to the patient by way of a unit dosage form including 30 mg of the corticosteroid.

In some embodiments of the foregoing aspect, the corticosteroid is administered to the patient by way of oral administration.

In some embodiments of any of the foregoing aspects, the patient does not have a history of transaminasemia or hyperbilirubinemia. In some embodiments, the patient does not have a history of any underlying liver disease.

In some embodiments of any of the foregoing aspects, the patient exhibits a symptom selected from feeding difficulties, failure to thrive, hypotonia, progressive weakness, respiratory distress, severe enlargement of the tongue, and thickening of the heart muscle.

In some embodiments of any of the foregoing aspects, the patient is undergoing GAA enzyme replacement therapy.

In some embodiments of any of the foregoing aspects, upon administering the viral vector to the patient, the patient exhibits endogenous GAA activity of from 50% to 200% of the endogenous GAA activity of a human of the same gender and similar body mass index that does not have Pompe disease.

In some embodiments of any of the foregoing aspects, upon administering the viral vector to the patient, the patient exhibits a reduction in glycogen in skeletal muscle, cardiac muscle, and/or neuronal tissue.

In some embodiments of the foregoing aspects, the method further including monitoring the patient for development of transaminasemia, hyperbilirubinemia, or one or more symptoms thereof.

In some embodiments, the patient is monitored for the development of transaminasemia, hyperbilirubinemia, or one or more symptoms thereof by evaluating a parameter in a blood sample obtained from the patient, wherein a finding that the parameter is above a reference level identifies the patient as having transaminasemia, hyperbilirubinemia, or one or more symptoms thereof. In some embodiments, the parameter includes the level of aspartate aminotransferase, alanine aminotransferase and/or bilirubin in the blood sample.

In some embodiments of any of the foregoing aspects, the patient is determined to exhibit transaminasemia or one or more symptoms thereof by a finding that the patient exhibits one or more transaminases in a liver function test that is increased relative to a reference level. In some embodiments, the one or more transaminases includes the level of aspartate aminotransferase and/or alanine aminotransferase.

In some embodiments of the foregoing aspects, the patient is determined to exhibit transaminasemia or one or more symptoms thereof by a finding that the patient exhibits an alanine transaminase level that is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L) in a liver function test.

In some embodiments of the foregoing aspects, upon administering the viral vector to the patient, the patient displays an aspartate aminotransferase level that is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L) in a liver function test.

In some embodiments of any of the foregoing aspects, the Pompe disease is late-onset Pompe disease (LOPD).

In another aspect, the disclosure provides a kit including a viral vector including a transgene encoding GAA and a package insert, wherein the package insert instructs a user of the kit to administer the viral vector to a patient having Pompe disease in accordance with the method of any one of the foregoing aspects.

In another aspect, the disclosure provides a kit including an anti-transaminitis agent and a package insert, wherein the package insert instructs a user of the kit to administer the anti-transaminitis agent to a patient to treat or prevent transaminasemia or hyperbilirubinemia in accordance with the method of any one of any one of the foregoing aspects.

### BRIEF DESCRIPTION OF THE DRAWINGS

The application file contains at least one drawing executed in color. Copies of this patent or patent application with color drawings will be provided by the Office upon request and payment of the necessary fee.

FIG. 1 is a schematic depicting the experimental outline of clinical trial FORTIS (NCT04174105), which is an ongoing multicenter, open-label, ascending dose Phase I/II first-in-human clinical trial to determine if an AAV vector encoding GAA, described herein, is safe and tolerable in adult subjects with Pompe disease. Subjects enrolled in FORTIS receive a one-time peripheral intravenous infusion of the vector described herein, followed by one year of frequent monitoring of clinical and biochemical endpoints including GAA activity and protein level in muscle and four years of long-term safety monitoring.

FIG. 2 is a set of graphs showing change in aspartate aminotransferase (AST), alanine aminotransferase (ALT), and total bilirubin (TBil) levels in individual subjects 2001 (“01”), 2002 (“02”), 2003 (“03”), and 2009 (“09”) over time relative to their treatment with a corticosteroid, prednisolone, as well as an AAV2/8 vector containing an acid alpha-glucosidase (GAA) transgene operably linked to a muscle creatine kinase (MCK) promoter. Abbreviations: L, liter; U/L, units per liter; ULM, upper limit of normal.

FIG. 3 is a graph showing change in AST, ALT, and TBil levels in subject 2001 over time relative to their treatment with a corticosteroid, prednisolone, as well as an AAV2/8 vector containing a GAA transgene operably linked to a MCK promoter (AAV-MCK-GAA). Abbreviations: L, liter; U/L, units per liter; ULM, upper limit of normal.

FIG. 4 is a set of graphs showing change in blood pressure as well as hemoglobin, platelets, creatine, and lactate dehydrogenase levels in individual subjects overtime relative to their treatment with an AAV2/8 vector containing a GAA transgene operably linked to a MCK promoter.

FIG. 5 is a set of graphs showing change in troponin I, troponin T, and B-type natriuretic peptide levels in individual subjects over time relative to their treatment with an AAV2/8 vector containing a GAA transgene operably linked to a MCK promoter.

FIG. 6 is a set of graphs showing change in creatine kinase and urinary Hex4 levels in individual subjects over time relative to their treatment with an AAV2/8 vector containing a GAA transgene operably linked to a MCK promoter (AAV-MCK-GAA).

FIG. 7 is a set of graphs showing the quantity of vector copies in the urine and saliva samples of individual subjects over time relative to their treatment with an AAV2/8 vector containing a GAA transgene operably linked to a MCK promoter.

FIG. 8 is a set of graphs showing the humoral immune response to treatment with an AAV2/8 vector containing a GAA transgene operably linked to a MCK promoter, as quantified (e.g., with an enzyme-linked immunosorbent assay (ELISA)) with an antibody titer test for anti-GAA or anti-AAV8 antibodies, respectively, of individual subjects over time relative to their treatment with an AAV2/8 vector containing a GAA transgene operably linked to a MCK promoter.

FIG. 9 is a graph showing the viral genome per diploid genome, as quantified with a vector copy number (VCN) assay in individual subjects before (pre-dose) and after (Week 12 and Week 36) their treatment with an AAV2/8 vector containing a GAA transgene operably linked to a MCK promoter.

FIG. 10 is a graph showing protein expression in target tissue, as quantified by GAA activity in the muscle biopsies of individual subjects overtime relative to their treatment with an AAV2/8 vector containing a GAA transgene operably linked to a MCK promoter.

FIG. 11 is a graph showing change in AST, ALT, and TBil levels in subject 2002 over time relative to their treatment with a corticosteroid, prednisolone, as well as an AAV2/8 vector containing a GAA transgene operably linked to a MCK promoter (AAV-MCK-GAA). Abbreviations: L, liter; U/L, units per liter; ULM, upper limit of normal.

FIG. 12 is a graph showing change in AST, ALT, and TBil levels in subject 2003 overtime relative to their treatment with a corticosteroid, prednisolone, as well as an AAV2/8 vector containing a GAA transgene operably linked to a MCK promoter (AAV-MCK-GAA). Abbreviations: L, liter; U/L, units per liter; ULM, upper limit of normal.

FIG. 13 is a graph showing change in AST, ALT, and TBil levels in subject 2009 overtime relative to their treatment with a corticosteroid, prednisolone, as well as an AAV2/8 vector containing a GAA transgene operably linked to a MCK promoter (AAV-MCK-GAA). Abbreviations: L, liter; U/L, units per liter; ULM, upper limit of normal.

### DEFINITIONS

As used herein, the term “about” refers to a value that is within 5% above or below the value being described. For example, “about 1×10¹³ vg/kg” as used in the context of a viral vector described herein includes quantities that are within 5% above or below 1×10¹³ vg/kg. Additionally, when used in the context of a list of numerical quantities, it is to be understood that the term “about,” when preceding a list of numerical quantities, applies to each individual quantity recited in the list. For example, “about 1×10¹³ vg/kg, 2×10¹³ vg/kg, or 3×10¹³ vg/kg” is to be construed as equivalent to individually reciting “about 1×10¹³ vg/kg,” “about 2×10¹³ vg/kg,” and “about 3×10¹³ vg/kg.”

As used herein in the context of a protein of interest, such as acid alpha-glucosidase (GAA), the term “activity” refers to the biological functionality that is associated with a wild-type form of the protein. For example, in the context of an enzyme, the term “activity” refers to the ability of the protein to effectuate substrate turnover in a manner that yields the product of a corresponding chemical reaction. Activity levels of enzymes, such as GAA, can be detected and quantitated, for example, using substrate turnover assays known in the art.

As used herein, the terms “administering,” “administration,” and the like refer to directly giving a patient a therapeutic agent (e.g., a viral vector) by any effective route. Exemplary routes of administration are described herein and include systemic administration routes, such as intravenous injection, as well as routes of administration directly to the central nervous system of the patient, such as by way of intrathecal injection or intracerebroventricular injection, among others.

As used herein, the terms “alanine aminotransferase” and “ALT” refer to a protein whose amino acid sequence comprises or consists of an amino acid sequence of a naturally occurring wild-type ALT protein (e.g., ALT1 and ALT2) as well as proteins whose amino acid sequence comprises or consists of an amino acid sequence of a naturally occurring allelic variants of ALT (GPT or GPT2 e.g., splice variants or allelic variants). Human GPT nucleic acid sequence is provided in NCBI RefSeq Acc. No. NM_005309.2, and an exemplary wild-type ALT1 amino acid sequence is provided in NCBI RefSeq Acc. No. NP_005300. Human GPT2 nucleic acid sequence is provided in NCBI RefSeq Acc. No. NM_001142466.2, and an exemplary wild-type ALT2 amino acid sequence is provided in NCBI RefSeq Acc. No. NP_001135938.1.

As used herein, the term “anti-transaminitis agent” refers to a substance, such as a small molecule (e.g., a corticosteroid) that acts to decrease, directly or indirectly, the level of one or more liver transaminases.

As used herein, the terms “aspartate aminotransferase” and “AST” refer to a protein whose amino acid sequence comprises or consists of an amino acid sequence of a naturally occurring wild-type AST protein as well as proteins whose amino acid sequence comprises or consists of an amino acid sequence of a naturally occurring allelic variants of AST (e.g., splice variants or allelic variants). Human AST nucleic acid sequence is provided in NCBI RefSeq Acc. No NM_002079.2, and an exemplary wild-type ASP amino acid sequence is provided in NCBI RefSeq Acc. No. NP_002070.1.

As used herein, the term “transaminasemia” and “transaminitis” refer synonymously to a condition where levels of liver enzymes, called transaminases, are elevated. “Transaminase elevation,” “elevated liver enzymes,” and “hypertransaminasemia” are other terms sometimes referring to the same thing.

As used herein, a “combination therapy” means that two (or more) different agents or treatments are administered to a subject as part of a defined treatment regimen for a particular disease or condition (e.g., a glycogen storage disorder). In some embodiments, a “combination therapy” may include a procedure. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be affected by any appropriate route including, but not limited to, oral routes, intravenous routes, intramuscular routes, and direct absorption through mucous membrane tissues. Therapeutic agents can be administered by the same route or by different routes. For example, a first therapeutic agent of the combination may be administered by intravenous injection while a second therapeutic agent of the combination may be administered enterally. In another example, an agent of the therapeutic combination may be administered by intravenous injection and a procedure of the therapeutic combination may be performed.

As used herein, the term “dose” refers to the quantity of a therapeutic agent, such as a viral vector described herein, that is administered to a subject at a particular instant for the treatment of a disorder or condition, such as to treat or ameliorate one or more symptoms of a glycogen storage disorder described herein (e.g., Pompe disease). A therapeutic agent as described herein may be administered in a single dose or in multiple doses over the course of a treatment period, as defined herein. In each case, the therapeutic agent may be administered using one or more unit dosage forms of the therapeutic agent, a term that refers to a one or more discrete compositions containing a therapeutic agent that collectively constitute a single dose of the agent. For instance, a single dose of 1×10¹³ vector genomes (vg) of a viral vector may be administered using, e.g., two 0.5×10¹³ vg unit dosage forms of the viral vector.

As used herein, the terms “effective amount,” “therapeutically effective amount,” and the like, when used in reference to a therapeutic composition, such as a vector construct described herein, refer to a quantity that, when administered to a subject (e.g., a mammal, such as a human) suffering from a disease or condition described herein, is sufficient to effect beneficial or desired results in treating the disease or condition. For example, in the context of treating glycogen storage disorders, such as Pompe disease, these terms refer to an amount of the composition sufficient to achieve a treatment response as compared to the response obtained without administration of the composition of interest. An “effective amount,” “therapeutically effective amount,” or the like, of a composition, such as a vector construct of the present disclosure, also include an amount that results in a beneficial or desired result in a subject as compared to a control.

As used herein, the terms “enzyme replacement therapy” or “ERT” refer to the administration to a subject (e.g., a mammalian subject, such as a human) suffering from a genetic loss-of-function disease of the protein that is naturally defective or deficient in the subject. For example, in the context of a subject having Pompe disease, enzyme replacement therapy refers to administration of GAA protein to such a subject. Typically, enzyme replacement therapy involves administration of the therapeutic protein to the subject chronically, over the course of multiple doses throughout the subject's life.

As used herein, the terms “express” and “expression” in the context of a gene refer to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. In the context of a gene that encodes a protein product, the terms “gene expression” and the like are used interchangeably with the terms “protein expression” and the like. Expression of a gene or protein of interest in a subject can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the subject. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. Additionally, two portions of a transcription regulatory element are operably linked to one another if they are joined such that the transcription-activating functionality of one portion is not adversely affected by the presence of the other portion. Two transcription regulatory elements may be operably linked to one another by way of a linker nucleic acid (e.g., an intervening non-coding nucleic acid) or may be operably linked to one another with no intervening nucleotides present.

As used herein, the term “hyperbilirubinemia” refers to a condition in which there is a higher-than-normal level of bilirubin in the blood. As used herein, the term “bilirubin” refers to a compound that occurs in the normal catabolic pathway that breaks down heme in vertebrates. This catabolism is a necessary process in the body's clearance of waste products that arise from the destruction of aged or abnormal red blood cells. As used herein, a “bilirubin test” refers to a measurement of the amount of bilirubin in a patient's blood.

As used herein, the term “level” refers to a level of a protein, as compared to a reference. The reference can be any useful reference, as defined herein. By a “decreased level” and an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2.0-fold, about 3.0-fold, about 3.5-fold, about 4.5-fold, about 5.0-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, μg/mL, or ng/mL) or percentage relative to total protein in a sample.

As used herein, the terms “liver function test” and “LFT” refers to a hepatic panel (e.g., a group of blood tests that provide information about the state of a patient's liver). A hepatic panel may include measurement of the level of gamma-glutamyl transferase, the level of alkaline phosphatase, the level of aspartate aminotransferase, the level of alanine aminotransferase, the level of albumin, the level of bilirubin, the prothrombin time, the activated partial thromboplastin time, or a combination thereof.

As used herein, the term “pharmaceutical composition” refers to a mixture containing a therapeutic compound to be administered to a subject, such as a mammal, e.g., a human, in order to prevent, treat or control a particular disease or condition affecting or that may affect the subject.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements. Additionally, the term “promoter” may refer to a synthetic promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of transgenes, vectors, and target cell types.

As used herein, a therapeutic agent is considered to be “provided” to a patient if the patient is directly administered the therapeutic agent or if the patient is administered a substance that is processed or metabolized in vivo to yield the therapeutic agent endogenously. For example, a patient, such as a patient having a glycogen storage disorder described herein, may be provided a nucleic acid molecule encoding a therapeutic protein (e.g., GAA) by direct administration of the nucleic acid molecule or by administration of a substance (e.g., viral vector or cell) that is processed in vivo to yield the desired nucleic acid molecule.

As used herein, the terms “subject,” “patient,” and “participant” refer to an organism that receives treatment for a particular disease or condition as described herein (such as a lysosomal storage disorder, e.g., Pompe disease). Examples of subjects, patients, and participants include mammals, such as humans, receiving treatment for a disease or condition described herein.

By a “reference” is meant any useful reference used to compare protein levels related to transaminasemia, hyperbilirubinemia, or one or more symptoms thereof. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a blood sample) from a subject not having transaminasemia, hyperbilirubinemia, or one or more symptoms thereof; a sample from a subject that is diagnosed with transaminasemia, hyperbilirubinemia, or one or more symptoms thereof; a sample from a subject that has been treated for transaminasemia, hyperbilirubinemia, or one or more symptoms thereof; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having transaminasemia, hyperbilirubinemia, or one or more symptoms thereof. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, or cells) isolated from a subject. The subject may be, for example, a patient suffering from a disease described herein, such as a lysosomal storage disorder (e.g., Pompe disease).

As used herein, the term “transgene” refers to a recombinant nucleic acid (e.g., DNA or cDNA) encoding a gene product (e.g., a gene product described herein). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s), and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the terms “treat” or “treatment” refer to therapeutic treatment, in which the object is to prevent or slow down (lessen) an undesired physiological change or disorder, such as the progression of a lysosomal storage disorder, such as Pompe disease, among others. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. In the context of lysosomal storage disorders, such as Pompe disease, treatment of a patient may manifest in one or more detectable changes, such as an increase in the concentration of GAA protein or nucleic acids (e.g., DNA or RNA, such as mRNA) encoding GAA, or an increase in GAA activity (e.g., by 5%, 10%, 20%, 30%, 40%, 50%, 60%, 70%, 80%, 90%, 100%, 200%, 300%, 400%, 500%, 600%, 700%, 800%, 900%, or more. The concentration of GAA protein may be determined using protein detection assays known in the art, including ELISA assays described herein. The concentration of GAA-encoding nucleic acids may be determined using nucleic acid detection assays (e.g., RNA Seq assays) described herein. Additionally, treatment of a patient suffering from a lysosomal storage disorder, such as Pompe disease, may manifest in improvements in a patient's muscle function (e.g., cardiac or skeletal muscle function) as well as improvements in muscle coordination.

As used herein, the term “Pompe disease” refers to the genetically inherited glycogen storage disorder that is caused by mutations of the GAA gene and is characterized by symptoms including mild to profound muscle weakness, hypotonia (diminished muscle tone), an enlarged liver, failure to gain weight and/or grow at the expected rate, trouble breathing, feeding problems, infections of the respiratory system, and/or problems with hearing. An exemplary wild-type human GAA amino acid sequence is provided in SEQ ID NO: 2, below.

As used herein, the term “vector” refers to a nucleic acid, e.g., DNA or RNA, that may function as a vehicle for the delivery of a gene of interest into a cell (e.g., a mammalian cell, such as a human cell), such as for purposes of replication and/or expression. Exemplary vectors useful in conjunction with the compositions and methods described herein are plasmids, DNA vectors, RNA vectors, virions, or another suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO94/11026, the disclosure of which is incorporated herein by reference. Expression vectors described herein contain a polynucleotide sequence as well as, e.g., additional sequence elements used for the expression of proteins and/or the integration of these polynucleotide sequences into the genome of a mammalian cell. Certain vectors that can be used for the expression of transgenes described herein include plasmids that contain regulatory sequences, such as promoter and enhancer regions, which direct gene transcription. Other useful vectors for expression of transgenes contain polynucleotide sequences that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, or nourseothricin.

As used herein in the context of a therapeutic protein, such as GAA, the use of the protein name refers to the gene encoding the protein or the corresponding protein product, depending upon the context, as will be appreciated by one of skill in the art. The term “GAA” includes wild-type forms of the GAA gene or protein, as well as variants (e.g., splice variants, truncations, concatemers, and fusion constructs, among others) of wild-type GAA proteins that retain therapeutic activity of the wild-type GAA protein, as well as nucleic acids encoding the same. Examples of such variants are proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.5% identity, or more) to an amino acid sequence of a wild-type GAA protein, such as SEQ ID NO: 2, below:

(SEQ ID NO: 2)
MGVRHPPCSHRLLAVCALVSLATAALLGHILLHDFLLVPRELSGSSPVLE
ETHPAHQQGASRPGPRDAQAHPGRPRAVPTQCDVPPNSRFDCAPDKAITQ
EQCEARGCCYIPAKQGLQGAQMGQPWCFFPPSYPSYKLENLSSSEMGYTA
TLTRTTPTFFPKDILTLRLDVMMETENRLHFTIKDPANRRYEVPLETPHV
HSRAPSPLYSVEFSEEPFGVIVRRQLDGRVLLNTTVAPLFFADQFLQLST
SLPSQYITGLAEHLSPLMLSTSWTRITLWNRDLAPTPGANLYGSHPFYLA
LEDGGSAHGVFLLNSNAMDVVLQPSPALSWRSTGGILDVYIFLGPEPKSV
VQQYLDVVGYPFMPPYWGLGFHLCRWGYSSTAITRQVVENMTRAHFPLDV
QWNDLDYMDSRRDFTFNKDGFRDFPAMVQELHQGGRRYMMIVDPAISSSG
PAGSYRPYDEGLRRGVFITNETGQPLIGKVWPGSTAFPDFTNPTALAWWE
DMVAEFHDQVPFDGMWIDMNEPSNFIRGSEDGCPNNELENPPYVPGVVGG
TLQAATICASSHQFLSTHYNLHNLYGLTEAIASHRALVKARGTRPFVISR
STFAGHGRYAGHWTGDVWSSWEQLASSVPEILQFNLLGVPLVGADVCGFL
GNTSEELCVRWTQLGAFYPFMRNHNSLLSLPQEPYSFSEPAQQAMRKALT
LRYALLPHLYTLFHQAHVAGETVARPLFLEFPKDSSTWTVDHQLLWGEAL
LITPVLQAGKAEVTGYFPLGTWYDLQTVPVEALGSLPPPPAAPREPAIHS
EGQWVTLPAPLDTINVHLRAGYIIPLQGPGLTTTESRQQPMALAVALTKG
GEARGELFWDDGESLEVLERGAYTQVIFLARNNTIVNELVRVTSEGAGLQ
LQKVTVLGVATAPQQVLSNGVPVSNFTYSPDTKVLDICVSLLMGEQFLVS
WC

Similarly, as used herein in the context of a transcription regulatory element, the term “MCK promoter” refers to a wild-type MCK promoter, such as a wild-type human or murine MCK promoter, as well as variants (e.g., variants containing insertions, deletions, and/or substitutions of one or more nucleic acid residues) to the extent that the promoter retains the ability to induce expression of an operably linked gene in a muscle and/or neuronal cell. An exemplary MCK promoter that may be used in conjunction with the compositions and methods of the disclosure is shown in SEQ ID NO: 1, below:

(SEQ ID NO: 1)
CCACTACGGGTCTAGGCTGCCCATGTAAGGAGGCAAGGCCTGGGGACACC
CGAGATGCCTGGTTATAATTAACCCAGACATGTGGCTGCCCCCCCCCCCC
CAACACCTGCTGCCTGAGCCTCACCCCCACCCCGGTGCCTGGGTCTTAGG
CTCTGTACACCATGGAGGAGAAGCTCGCTCTAAAAATAACCCTGTCCCTG
GTGGATCCCCTGCATGCCCAATCAAGGCTGTGGGGGACTGAGGGCAGGCT
GTAACAGGCTTGGGGGCCAGGGCTTATACGTGCCTGGGACTCCCAAAGTA
TTACTGTTCCATGTTCCCGGCGAAGGGCCAGCTGTCCCCCGCCAGCTAGA
CTCAGCACTTAGTTTAGGAACCAGTGAGCAAGTCAGCCCTTGGGGCAGCC
CATACAAGGCCATGGGGCTGGGCAAGCTGCACGCCTGGGTCCGGGGTGGG
CACGGTGCCCGGGCAACGAGCTGAAAGCTCATCTGCTCTCAGGGGCCCCT
CCCTGGGGACAGCCCCTCCTGGCTAGTCACACCCTGTAGGCTCCTCTATA
TAACCCAGGGGCACAGGGGCTGCCCCCGGGTCAC

### Chemical Terms

The chemical terminology used herein is for the purpose of describing various aspects and embodiments of the disclosure and is not intended to be limiting.

In the following chemical definitions, a notation in which an integral number immediately follows an atomic symbol indicates the quantity of atoms of that element that are present in a particular chemical moiety. As will be understood, other atoms, such as hydrogen atoms, or substituent groups described herein, may be present, as necessary, to satisfy the valence of a particular atom. For example, an unsubstituted “C₂ alkyl group” has the formula —CH₂CH₃. When used in conjunction with the groups defined herein, a reference to a number of carbon atoms includes the divalent carbon in acetal and ketal groups but does not include the carbonyl carbon in acyl, ester, carbonate, amide, or carbamate groups. A reference to a number of oxygen, nitrogen, or sulfur atoms in a heteroaryl group only includes those atoms that form a part of a heterocyclic ring.

As used herein, a phrase of the form “optionally substituted X” (e.g., optionally substituted alkyl) is intended to be equivalent to “X, wherein X is optionally substituted” (e.g., “alkyl, wherein the alkyl is optionally substituted”). It is not intended to mean that the feature “X” (e.g., alkyl) per se is optional. As described herein, certain compounds may contain one or more “optionally substituted” moieties. In general, the term “substituted”, whether preceded by the term “optionally” or not, means that one or more hydrogens of the designated moiety are replaced with a suitable substituent, such as any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents that may be used in conjunction with the compounds of the disclosure are preferably those that result in the formation of stable or chemically feasible compounds. The term “stable,” as used herein, refers to compounds that are not substantially altered when subjected to conditions that allow for their production, detection, and, in certain embodiments, recovery, purification, and use for one or more of the purposes disclosed herein.

As used herein, the term “aliphatic” refers to a saturated or unsaturated, straight, branched, or cyclic hydrocarbon. The term “aliphatic” includes, but is not limited to, alkyl, alkenyl, alkynyl, cycloalkyl, cycloalkenyl, and cycloalkynyl moieties, and thus incorporates each of these definitions. In some embodiments, “aliphatic” is used to indicate those aliphatic groups having from 1 to 20 carbon atoms. The aliphatic chain may be, for example, mono-unsaturated, di-unsaturated, tri-unsaturated, or polyunsaturated, or alkynyl. Unsaturated aliphatic groups can be in a cis or trans configuration. In some embodiments, the aliphatic group contains from 1 to about 12 carbon atoms, such as from 1 to about 6 carbon atoms or from 1 to about 4 carbon atoms. In some embodiments, the aliphatic group contains from 1 to about 8 carbon atoms. In some embodiments, the aliphatic group is C₁-C₂, C₁-C₃, C₁-C₄, C₁-C₅, or C₁-C₆. The specified ranges used herein indicate an aliphatic group having each member of the range described as an independent species. For example, the term “C₁-C₆ aliphatic” as used herein indicates a straight or branched alkyl, alkenyl, or alkynyl group having from 1, 2, 3, 4, 5, or 6 carbon atoms and is intended to mean that each of these is described as an independent species. For example, the term “C₁-C₄ aliphatic” as used herein indicates a straight or branched alkyl, alkenyl, or alkynyl group having from 1, 2, 3, or 4 carbon atoms and is intended to mean that each of these is described as an independent species. In some embodiments, the aliphatic group is substituted with one or more functional groups that results in the formation of a stable moiety.

As used herein, the term “heteroaliphatic” refers to an aliphatic moiety that contains at least one heteroatom in its chain, such as an amine, carbonyl, carboxy, oxo, thio, phosphate, phosphonate, nitrogen, phosphorus, silicon, or boron atom in place of a carbon atom. In some embodiments, the heteroatom present is nitrogen. In some embodiments, the heteroatom present is oxygen. In some embodiments, the heteroatom present is sulfur. The term “heteroaliphatic” includes, but is not limited to, heteroalkyl, heteroalkenyl, heteroalkynyl, heterocycloalkyl, heterocycloalkenyl, and heterocycloalkynyl moieties. In some embodiments, “heteroaliphatic” is used to indicate a heteroaliphatic group (cyclic, acyclic, substituted, unsubstituted, branched or unbranched) having from 1 to 20 carbon atoms. In some embodiments, the heteroaliphatic group is optionally substituted in a manner that results in the formation of a stable moiety. Nonlimiting examples of heteroaliphatic moieties are polyethylene glycol, polyalkylene glycol, amide, polyamide, glycolide, polylactide, polyglycolide, thioether, ether, alkyl-heterocycle-alkyl, —O-alkyl-O-alkyl, and alkyl-O-haloalkyl.

As used herein, the term “acyl” refers to a carbonyl substituent, such as a carbonyl substituent in which the carbonyl carbon is bound to an alkyl group, an alkenyl group, an alkynyl group, an optionally substituted oxygen moiety, an optionally substituted nitrogen moiety, and the like. Exemplary acyl groups include, without limitation, formyl (i.e., a carboxyaldehyde group), acetyl, trifluoroacetyl, propionyl, and butanoyl. Exemplary unsubstituted acyl groups include from 1 to 6, from 1 to 11, or from 1 to 21 carbons.

As used herein, the term “acyloxy” refers to the chemical moiety —OC(O)R in which R is C₁-C₆ alkyl, aryl, heteroaryl, C₁-C₆ alkyl aryl, or C₁-C₆ alkyl heteroaryl.

As used herein, the term “alkyl” refers to a branched or straight-chain monovalent saturated aliphatic hydrocarbon radical of 1 to 20 carbon atoms (e.g., 1 to 16 carbon atoms, 1 to 10 carbon atoms, 1 to 6 carbon atoms, or 1 to 3 carbon atoms). As used herein, the term “alkylene” refers to a divalent alkyl group.

As used herein, the term “alkenyl,” whether recited alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6, or 2 carbon atoms). As used herein, the term “alkenylene” refers to a divalent alkenyl group.

As used herein, the term “alkynyl,” whether recited alone or in combination with other groups, refers to a straight chain or branched hydrocarbon residue having a carbon-carbon triple bond and having 2 to 20 carbon atoms (e.g., 2 to 16 carbon atoms, 2 to 10 carbon atoms, 2 to 6, or 2 carbon atoms). As used herein, the term “alkynylene” refers to a divalent alkynyl group.

As used herein, the term “amino” represents —N(R^{N1})₂, wherein each R^{N1} is, independently, H, OH, NO₂, N(R^{N2})₂, SO₂OR^{N2}, SO₂R^{N2}, SOR^{N2}, an N-protecting group, alkyl, alkoxy, aryl, arylalkyl, cycloalkyl, acyl (e.g., acetyl, trifluoroacetyl, or others described herein), wherein each of these recited R^{N1} groups can be optionally substituted; or two R^{N1} combine to form an alkylene or heteroalkylene, and wherein each R^{N2} is, independently, H, alkyl, or aryl. The amino groups of the compounds described herein can be an unsubstituted amino (i.e., —NH₂) or a substituted amino (i.e., —N(R^{N1})₂).

As used herein, the term “aryl” refers to an aromatic mono- or polycarbocyclic radical of, e.g., 6 to 12, carbon atoms having at least one aromatic ring. Examples of such groups include, but are not limited to, phenyl, naphthyl, 1,2,3,4-tetrahydronaphthyl, 1,2-dihydronaphthyl, indanyl, and 1H-indenyl.

As used herein, the term “arylalkyl” represents an alkyl group substituted with an aryl group. Exemplary unsubstituted arylalkyl groups are from 7 to 30 carbons (e.g., from 7 to 16 or from 7 to 20 carbons, such as C₁-C₆ alkyl C₆-C₁₀ aryl, C₁-C₁₀ alkyl C₆-C₁₀ aryl, or C₁-C₂₀ alkyl C₆-C₁₀ aryl), such as, benzyl and phenethyl. In some embodiments, the alkyl and the aryl each can be further substituted with 1, 2, 3, or 4 substituent groups as defined herein for the respective groups.

As used herein, the term “bridged cyclyl” refers to a bridged polycyclic group of 5 to 20 atoms, containing from 1 to 3 bridges. Bridged cyclyl includes bridged carbocyclyl (e.g., norbornyl) and bridged heterocyclyl (e.g., 1,4-diazabicyclo[2.2.2]octane).

As used herein, the term “carbocyclyl” refers to a non-aromatic C₃-C₁₂, monocyclic or polycyclic (e.g., bicyclic or tricyclic) structure in which the rings are formed by carbon atoms. Carbocyclyl structures include cycloalkyl groups (e.g., cyclohexyl) and unsaturated carbocyclyl radicals (e.g., cyclohexenyl). Polycyclic carbocyclyl includes spirocyclic carbocyclyl, bridged carbocyclyl, and fused carbocyclyl. As used herein, the term “carbocyclylene” refers to a divalent carbocyclyl group.

As used herein, the term “cycloalkyl” refers to a saturated, non-aromatic, monovalent mono- or polycarbocyclic radical of 3 to 10, preferably 3 to 6 carbon atoms. This term is further exemplified by radicals such as cyclopropyl, cyclobutyl, cyclopentyl, cyclohexyl, cycloheptyl, norbornyl, and adamantyl.

As used herein, the terms “halo” and “halogen” mean a fluorine (fluoro), chlorine (chloro), bromine (bromo), or iodine (iodo) radical.

As used herein, the term “heteroalkyl” refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers to alkyl-O— (e.g., methoxy and ethoxy), and an “alkylamino” which, as used herein, refers to —N(alkyl)R^{Na}, where R^{Na} is H or alkyl (e.g., methylamino). As used herein, the term “heteroalkylene” refers to a divalent heteroalkyl group.

As used herein, the term “heteroalkenyl” refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers to alkenyl-O—. As used herein, the term “heteroalkenylene” refers to a divalent heteroalkenyl group.

As used herein, the term “heteroalkynyl” refers to an alkynyl group, as defined herein, in which one or more of the constituent carbon atoms have been replaced by nitrogen, oxygen, or sulfur. In some embodiments, the heteroalkynyl group is further substituted with 1, 2, 3, or 4 substituent groups as described herein for alkynyl groups. Examples of heteroalkynyl groups are an “alkynoxy” which, as used herein, refers to alkynyl-O—. As used herein, the term “heteroalkynylene” refers to a divalent heteroalkynyl group.

As used herein, the term “heteroaryl” refers to an aromatic monocyclic or polycyclic structure of 5 to 12 atoms having at least one aromatic ring containing 1, 2, or 3 ring atoms selected from nitrogen, oxygen, and sulfur, with the remaining ring atoms being carbon. In some embodiments, one or two ring carbon atoms of the heteroaryl group are replaced with a carbonyl group. Examples of heteroaryl groups are pyridyl, pyrazoyl, benzooxazolyl, benzoimidazolyl, benzothiazolyl, imidazolyl, oxaxolyl, and thiazolyl. As used herein, the term “heteroarylene” refers to a divalent heteroaryl group.

As used herein, the term “heteroarylalkyl” represents an alkyl group substituted with a heteroaryl group. Exemplary unsubstituted heteroarylalkyl groups are from 7 to 30 carbons (e.g., from 7 to 16 or from 7 to 20 carbons, such as C₁-C₆ alkyl C₂-C₉ heteroaryl, C₁-C₁₀ alkyl C₂-C₉ heteroaryl, or C₁-C₂₀ alkyl C₂-C₉ heteroaryl). In some embodiments, the alkyl and the heteroaryl each can be further substituted with 1, 2, 3, or 4 substituent groups as defined herein for the respective groups.

As used herein, the term “heterocyclyl” refers a monocyclic or polycyclic radical (e.g., bicyclic or tricyclic) having 3 to 12 atoms having at least one non-aromatic ring containing 1, 2, 3, or 4 ring atoms selected from N, O, or S, and no aromatic ring containing any N, O, or S atoms. Polycyclic heterocyclyl includes spirocyclic heterocyclyl, bridged heterocyclyl, and fused heterocyclyl. Examples of heterocyclyl groups include, but are not limited to, morpholinyl, thiomorpholinyl, furyl, piperazinyl, piperidinyl, pyranyl, pyrrolidinyl, tetrahydropyranyl, tetrahydrofuranyl, and 1,3-dioxanyl. As used herein, the term “heterocyclylene” refers to a divalent heterocyclyl group.

As used herein, the term “heterocyclylalkyl” represents an alkyl group substituted with a heterocyclyl group. Exemplary unsubstituted heterocyclylalkyl groups are from 7 to 30 carbons (e.g., from 7 to 16 or from 7 to 20 carbons, such as C₁-C₆ alkyl C₂-C₉ heterocyclyl, C₁-C₁₀ alkyl C₂-C₉ heterocyclyl, or C₁-C₂₀ alkyl C₂-C₉ heterocyclyl). In some embodiments, the alkyl and the heterocyclyl each can be further substituted with 1, 2, 3, or 4 substituent groups as defined herein for the respective groups.

As used herein, the term “hydroxyalkyl” refers to an alkyl group substituted with an —OH group.

As used herein, the term “hydroxyl” refers to an —OH group.

As used herein, the term “imine” refers to a ═NR^{N} group, where RN is, e.g., H or alkyl.

As used herein, the term “N-protecting group” refers to those groups intended to protect an amino group against undesirable reactions during synthetic procedures. Commonly used N-protecting groups are disclosed in Greene, “Protective Groups in Organic Synthesis,” 3rd Edition (John Wiley & Sons, New York, 1999). N-protecting groups include, but are not limited to, acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, 4-nitrobenzoyl, and chiral auxiliaries such as protected or unprotected D, L, or D, L-amino acids such as alanine, leucine, and phenylalanine; sulfonyl-containing groups such as benzenesulfonyl, and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-20 dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, and phenylthiocarbonyl, arylalkyl groups such as benzyl, triphenylmethyl, and benzyloxymethyl, and silyl groups, such as trimethylsilyl. Preferred N-protecting groups are alloc, formyl, acetyl, benzoyl, pivaloyl, t-butylacetyl, alanyl, phenylsulfonyl, benzyl, t-butyloxycarbonyl (Boc), and benzyloxycarbonyl (Cbz).

As used herein, the term “nitro” refers to an —NO₂ group.

As used herein, the term “oxo” refers to an ═O group.

As used herein, the term “sulfonyl” refers to chemical moiety —SO₂—R in which R is hydrogen, aryl, heteroaryl, C₁-C₆ alkyl, C₁-C₆ alkyl substituted with one or more halogens, such as a —SO₂—CF₃ substituent, C₁-C₆ alkyl aryl, or C₁-C₆ alkyl heteroaryl.

As used herein, the term “sulfonylamino” refers to the chemical moiety —NRSO₂—R′ in which each of R and R′ is independently hydrogen, C₁-C₆ alkyl, aryl, heteroaryl, C₁-C₆ alkyl aryl, or C₁-C₆ alkyl heteroaryl.

As used herein, the term “sulfonyloxy” refers to the chemical moiety —OSO₂—R in which R is hydrogen, C₁-C₆ alkyl, C₁-C₆ alkyl substituted with one or more halogens, such as a —OSO₂—CF₃ substituent, aryl, heteroaryl, C₁-C₆ alkyl aryl, or C₁-C₆ alkyl heteroaryl.

As used herein, the term “thiol” refers to an —SH group.

The alkyl, alkenyl, alkynyl, heteroalkyl, heteroalkenyl, heteroalkynyl, carbocyclyl (e.g., cycloalkyl), aryl, heteroaryl, and heterocyclyl groups described herein may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halogen (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, oxo, sulfonyl, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

### Depictions of Chemical Structures

Compounds of the disclosure may have one or more asymmetric carbon atoms and may exist in the form of optically pure enantiomers, mixtures of enantiomers (e.g., racemates), optically pure diastereoisomers, mixtures of diastereoisomers, diastereoisomeric racemates, or mixtures of diastereoisomeric racemates. The optically active forms can be obtained, for example, by resolution of the racemates, by asymmetric synthesis or asymmetric chromatography (chromatography with a chiral adsorbent or eluant). Accordingly, the compounds disclosed herein may exist in various stereoisomeric forms.

Stereoisomers are compounds that differ only in their spatial arrangement. Enantiomers are pairs of stereoisomers whose mirror images are not superimposable, most commonly because they contain an asymmetrically substituted carbon atom that acts as a chiral center. The term “enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms.

Enantiomers of a compound can be prepared, for example, by separating an enantiomer from a racemate using one or more well-known techniques and methods, such as, for example, chiral chromatography and separation methods based thereon. The terms “racemate” and “racemic mixture” refer to a compound containing two enantiomers, wherein such mixtures exhibit no optical activity, i.e., they do not rotate the plane of polarized light. The term “geometric isomer” refers to isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side of the carbon-carbon double bond) configuration. “R,” “S,” “S*,” “R*,” “E,” “Z,” “cis,” and “trans,” indicate configurations relative to the core molecule.

When the stereochemistry of a compound disclosed herein is named or depicted by structure, the named or depicted stereoisomer is greater than 50% by weight (e.g., at least 60%, 70%, 80%, 90%, 99%, or 99.9% by weight) relative to its other stereoisomers. For example, when a single enantiomer is named or depicted by structure, the depicted or named enantiomer is greater than 50% by weight (e.g., at least 60%, 70%, 80%, 90%, 99%, or 99.9% by weight) optically pure. Similarly, when a single diastereomer is named or depicted by structure, the depicted or named diastereomer is greater than 50% by weight (e.g., at least 60%, 70%, 80%, 90%, 99%, or 99.9% by weight) pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers.

Additionally, when the stereochemistry of a compound disclosed herein is named or depicted by structure, the named or depicted stereoisomer is greater than 50% by mole fraction (e.g., at least 60%, 70%, 80%, 90%, 99%, or 99.9% by mole fraction) relative to its other stereoisomers. For example, when a single enantiomer is named or depicted by structure, the depicted or named enantiomer is greater than 50% by mole fraction (e.g., at least 60%, 70%, 80%, 90%, 99%, or 99.9% by mole fraction) relative to the other enantiomer. When a single diastereomer is named or depicted by structure, the depicted or named diastereomer is greater than 50% by mole fraction (e.g., at least 60%, 70%, 80%, 90%, 99%, or 99.9% by mole fraction) relative to the other diastereomer(s) of the indicated compound. For enantiomeric compounds, percent purity by mole fraction is calculated as the ratio of the molar quantity of the enantiomer of interest relative to the sum of the molar quantities of (i) the enantiomer of interest and (ii) the optical isomer. Similarly, for diastereomeric compounds, percent purity by moles fraction is calculated as the ratio of the molar quantity of the diastereomer of interest relative to the total molar quantities of all diastereomers present for the indicated compound.

When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer.

When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The present disclosure embraces all of these forms.

### Polymorphic Compounds

As will be appreciated by one of skill in the art, many chemical entities can adopt a variety of different solid forms such as, for example, amorphous forms or crystalline forms (e.g., polymorphs, hydrates, solvate). In some embodiments, compounds of the present disclosure may be utilized in any such form, including in any solid form. In some embodiments, compounds described or depicted herein may be provided or utilized in hydrate or solvate form.

### DETAILED DESCRIPTION

The present disclosure provides compositions and methods that can be used for treating glycogen storage disorders, particularly, type II glycogen storage disorder, also known as Pompe disease. In accordance with the compositions and methods described herein, a patient (e.g., a human patient) having Pompe disease may be administered a viral vector, such as an adeno-associated viral (AAV) vector, that contains a transgene encoding acid alpha-glucosidase (GAA). The AAV vector may be, for example, a pseudotyped AAV vector, such as an AAV vector containing AAV2 inverted terminal repeats packaged within capsid proteins from AAV8 (AAV2/8) or AAV9 (AAV2/9). In some embodiments, the transgene is operably linked to a transcription regulatory element, such as a promoter that induces gene expression in a muscle cell and/or a neuronal cell. Exemplary promoters that may be used in conjunction with the compositions and methods of the disclosure are a muscle creatine kinase (MCK) promoter and cytomegalovirus (CMV) promoter, among others. The viral vector may be administered in combination with an anti-transaminitis agent (e.g., a corticosteroid).

The present disclosure is based, at least in part, on the discovery of methods of therapeutic and prophylactic treatment that address a significant medical need associated with the existing gene therapy approaches involving the delivery of GAA to patients in need thereof (e.g., patients with Pompe disease). The present disclosure is also based, in part, on the discovery that the existing gene therapy approaches involving the delivery of GAA to patients in need thereof (e.g., patients with Pompe disease) are associated with risks, including liver-related syndromes, such as transaminasemia, hyperbilirubinemia, or one or more symptoms thereof. More particularly, the present invention relates to the discovery of a method including administration of a viral vector including a transgene encoding GAA (e.g., the viral vector) and an anti-transaminitis agent (e.g., a corticosteroid, a bile acid, a farnesoid X receptor (FXR) ligand, a fibroblast growth factor 19 (FGF-19) mimetic, a Takeda-G-protein-receptor-5 (TGR5) agonist, a peroxisome proliferator-activated receptor (PPAR) agonist, a PPAR-alpha agonist, a PPAR-delta agonist, a dual PPAR-alpha and PPAR-delta agonist, an apical sodium-dependent corticosteroid transporter (ASBT) inhibitor, an immunomodulatory drug, an antifibrotic therapy, and a nicotinamide adenine dinucleotide phosphate oxidase (NOX) inhibitor) as a prophylactic treatment for liver syndromes associated with the existing gene therapy approaches involving the delivery of GAA to patients in need thereof (e.g., patients with Pompe disease). In some embodiments, the anti-transaminitis agent is a corticosteroid. In some embodiments, the corticosteroid is prednisolone.

In some embodiments, the disclosure describes a method of treating or preventing transaminasemia or hyperbilirubinemia in a human patient that has Pompe disease and who has been administered a therapeutically effective amount of a viral vector including a transgene encoding GAA includes administering to the patient an anti-transaminitis agent.

In some embodiments, the disclosure describes a method of treating Pompe disease in a human patient in need thereof and who has been previously administered an anti-transaminitis agent, the method including administering to the patient a therapeutically effective amount of a viral vector including a transgene encoding GAA.

In some embodiments, the disclosure describes a method of reducing glycogen accumulation in muscle tissue and/or in neuronal tissue in a human patient diagnosed as having Pompe disease, the method including administering to the patient a viral vector including a transgene encoding GAA and (an anti-transaminitis agent.

In some embodiments, the disclosure describes a method of improving pulmonary function in a human patient diagnosed as having Pompe disease, the method including administering to the patient a viral vector including a transgene encoding GAA and (an anti-transaminitis agent.

In some embodiments, the disclosure describes a method of increasing GAA expression in a human patient diagnosed as having Pompe disease, the method including administering to the patient a viral vector including a transgene encoding GAA and (an anti-transaminitis agent.

In some embodiments, the disclosure describes a method of reducing glycogen accumulation in muscle tissue and/or in neuronal tissue in a human patient diagnosed as having Pompe disease and who has been previously administered an anti-transaminitis agent, the method including administering to the patient a therapeutically effective amount of a viral vector including a transgene encoding GAA.

In some embodiments, the disclosure describes a method of improving pulmonary function in a human patient diagnosed as having Pompe disease and who has been previously administered an anti-transaminitis agent, the method including administering to the patient a therapeutically effective amount of a viral vector including a transgene encoding GAA.

In some embodiments, the disclosure describes a method of increasing GAA expression in a human patient diagnosed as having Pompe disease and who has been previously administered an anti-transaminitis agent, the method including administering to the patient a therapeutically effective amount of a viral vector including a transgene encoding GAA.

The sections that follow provide a description of therapeutic agents and parameters for assessing transaminasemia, hyperbilirubinemia, or one or more symptoms thereof that result in the administration of an anti-transaminitis agent described herein. The following sections also describe various transduction agents that may be used in conjunction with the compositions and methods of the disclosure.

### Methods of Treatment

### Pompe Disease

Pompe disease (also known as glycogen storage disease type II, or GSD II) is caused by deficiency of the lysosomal enzyme GAA. The disease is an inborn error of metabolism in which a GAA deficiency ultimately results in glycogen accumulation in all tissues, especially striated muscle cells. In addition, the effect of glycogen accumulation within the central nervous system and its effect on skeletal muscle function have been documented.

Three clinical forms of this disorder are known: infantile, juvenile, and adult. Infantile Pompe disease has its onset shortly after birth and presents with progressive muscular weakness and cardiac failure. Infantile forms of Pompe are also characterized by a rapid development of cardiomyopathy, and patients often display myopathy and neuropathy leading to death typically in the first year of life. Symptoms in adult and juvenile patients occur later in life, and skeletal muscles and neurons are primarily involved. Patients exhibiting this form of Pompe disease eventually die due to respiratory insufficiency. Patients may exceptionally survive for more than six decades. There is a correlation between the severity of the disease and the residual acid α-glucosidase activity, the activity being 10-20% of normal in late onset and less than 2% in early onset forms of the disease.

In some embodiments, the patient is a newborn (e.g., 0-4 months old), an infant (e.g., 0-5 months old) or a toddler (e.g., 6-12 months old) at the time of administration of the viral vector.

In some embodiments, the patient is a newborn (e.g., 0-4 months old) at the time of administration of the viral vector. For example, in some embodiments, the patient is a newborn that is about 0 to about 4 months old (e.g., 0 months old to about 4 months old, 1 month old to about 4 months old, 2 months old to about 4 months old, or 3 months old to about 4 months old). In some embodiments, the patient is 0 months old. In some embodiments, the patient is 1 month old. In some embodiments, the patient is 2 months old. In some embodiments, the patient is 3 months old. In some embodiments, the patient is 4 months old.

In some embodiments, the patient is a newborn (e.g., less than about 4 months old) at the time of administration of the viral vector. For example, in some embodiments, the patient is a newborn that is less than about 4 months old. In some embodiments, the patient is less than about 4 months old. In some embodiments, the patient is less than about 3 months old. In some embodiments, the patient is less than about 2 months old. In some embodiments, the patient is less than about 1 month old.

In some embodiments, the patient is an infant (e.g., 0-5 months old) at the time of administration of the viral vector. For example, in some embodiments, the patient is an infant that is about 0 months old to about 5 months old (e.g., 0 months old to about 5 months old, 1 month old to about 5 months old, 2 months old to about 5 months old, 3 months old to about 5 months old, or 4 months old to about 5 months old). In some embodiments, the patient is 0 months old. In some embodiments, the patient is 1 month old. In some embodiments, the patient is 2 months old. In some embodiments, the patient is 3 months old. In some embodiments, the patient is 4 months old. In some embodiments, the patient is 3 months old. In some embodiments, the patient is 5 months old.

In some embodiments, the patient is an infant (e.g., less than about 5 months old) at the time of administration of the viral vector. For example, in some embodiments, the patient is an infant that is less than about 5 months old. In some embodiments, the patient is less than about 5 months old. In some embodiments, the patient is less than about 4 months old. In some embodiments, the patient is less than about 3 months old. In some embodiments, the patient is less than about 2 months old. In some embodiments, the patient is less than about 1 month old.

In some embodiments, the patient is a toddler (e.g., 6-12 months old) at the time of administration of the viral vector. For example, in some embodiments, the patient is an infant that is about 6 months old to about 12 months old (e.g., 6 months old to about 12 months old, 7 months old to about 12 months old, 8 months old to about 12 months old, 9 months old to about 12 months old, 10 months old to about 12 months old, or 11 months old to about 12 months old). In some embodiments, the patient is 6 months old. In some embodiments, the patient is 7 months old. In some embodiments, the patient is 8 months old. In some embodiments, the patient is 9 months old.

In some embodiments, the patient is 10 months old. In some embodiments, the patient is 11 months old. In some embodiments, the patient is 12 months old.

In some embodiments, the patient is a toddler (e.g., less than about 12 months old) at the time of administration of the viral vector. For example, in some embodiments, the patient is a toddler that is less than about 12 months old. In some embodiments, the patient is less than about 12 months old. In some embodiments, the patient is less than about 11 months old. In some embodiments, the patient is less than about 10 months old. In some embodiments, the patient is less than about 9 months old. In some embodiments, the patient is less than about 8 months old. In some embodiments, the patient is less than about 7 months old. In some embodiments, the patient is less than about 6 months old. In some embodiments, the patient is less than about 5 months old. In some embodiments, the patient is less than about 4 months old. In some embodiments, the patient is less than about 3 months old. In some embodiments, the patient is less than about 2 months old. In some embodiments, the patient is less than about 1 month old.

In some embodiments, the patient was from about 1 month old to about 1 year old (e.g., about 1 month old to about 1 year old, about 2 months old to about 1 year old, about 3 months old to about 1 year old, about 4 months old to about 1 year old, about 5 months old to about 1 year old, or about 6 months old to about 1 year old) at the time of administration of the viral vector.

In some embodiments, the patient was from about 1 month old to about 5 years old (e.g., about 1 month old to about 5 years old, about 2 months old to about 5 years old, about 3 months old to about 5 years old, about 4 months old to about 5 years old, about 5 months old to about 5 years old, about 6 months old to about 5 years old, about 1 year old to about 5 years old, about 2 years old to about 5 years old, about 3 years old to about 5 years old, or about 4 years old to about 5 years old) at the time of administration of the transgene or viral vector.

In some embodiments, the patient is 5 years old or older (e.g., 5 years old or older, 6 years old or older, 7 years old or older, 8 years old or older, 9 years old or older, 10 years old or older, 15 years old or older, 20 years old or older, 25 years old or older, 30 years old or older, 40 years old or older, 50 years old or older, 60 years old or older, or 70 years old or older) at the time of administration of the transgene or viral vector. For example, in some embodiments, the patient is greater than 5 years old. In some embodiments, the patient is greater than 6 years old. In some embodiments, the patient is greater than 7 years old. In some embodiments, the patient is greater than 8 years old. In some embodiments, the patient is greater than 9 years old. In some embodiments, the patient is greater than 10 years old. In some embodiments, the patient is greater than 15 years old. In some embodiments, the patient is greater than 20 years old. In some embodiments, the patient is greater than 25 years old. In some embodiments, the patient is greater than 30 years old. In some embodiments, the patient is greater than 35 years old. In some embodiments, the patient is greater than 40 years old. In some embodiments, the patient is greater than 45 years old. In some embodiments, the patient is greater than 50 years old. In some embodiments, the patient is greater than 55 years old. In some embodiments, the patient is greater than 60 years old. In some embodiments, the patient is greater than 70 years old.

The patient may be of any age.

In some embodiments, the patient is male.

In some embodiments, the patient is female.

### Human Acid Alpha-Glucosidase

The amino acid sequence of an exemplary wild-type GAA is set forth in SEQ ID NO: 2, below:

(SEQ ID NO: 2)
MGVRHPPCSHRLLAVCALVSLATAALLGHILLHDFLLVPRELSGSSPVLE
ETHPAHQQGASRPGPRDAQAHPGRPRAVPTQCDVPPNSRFDCAPDKAITQ
EQCEARGCCYIPAKQGLQGAQMGQPWCFFPPSYPSYKLENLSSSEMGYTA
TLTRTTPTFFPKDILTLRLDVMMETENRLHFTIKDPANRRYEVPLETPHV
HSRAPSPLYSVEFSEEPFGVIVRRQLDGRVLLNTTVAPLFFADQFLQLST
SLPSQYITGLAEHLSPLMLSTSWTRITLWNRDLAPTPGANLYGSHPFYLA
LEDGGSAHGVFLLNSNAMDVVLQPSPALSWRSTGGILDVYIFLGPEPKSV
VQQYLDVVGYPFMPPYWGLGFHLCRWGYSSTAITRQVVENMTRAHFPLDV
QWNDLDYMDSRRDFTFNKDGFRDFPAMVQELHQGGRRYMMIVDPAISSSG
PAGSYRPYDEGLRRGVFITNETGQPLIGKVWPGSTAFPDFTNPTALAWWE
DMVAEFHDQVPFDGMWIDMNEPSNFIRGSEDGCPNNELENPPYVPGVVGG
TLQAATICASSHQFLSTHYNLHNLYGLTEAIASHRALVKARGTRPFVISR
STFAGHGRYAGHWTGDVWSSWEQLASSVPEILQFNLLGVPLVGADVCGFL
GNTSEELCVRWTQLGAFYPFMRNHNSLLSLPQEPYSFSEPAQQAMRKALT
LRYALLPHLYTLFHQAHVAGETVARPLFLEFPKDSSTWTVDHQLLWGEAL
LITPVLQAGKAEVTGYFPLGTWYDLQTVPVEALGSLPPPPAAPREPAIHS
EGQWVTLPAPLDTINVHLRAGYIIPLQGPGLTTTESRQQPMALAVALTKG
GEARGELFWDDGESLEVLERGAYTQVIFLARNNTIVNELVRVTSEGAGLQ
LQKVTVLGVATAPQQVLSNGVPVSNFTYSPDTKVLDICVSLLMGEQFLVS
WC

Exemplary genes encoding a GAA polypeptide that may be used in conjunction with the compositions and methods described herein include genes encoding the wild-type GAA protein set forth in SEQ ID NO: 2, as well as functional GAA enzymes having at least 85% identical (e.g., 85%, 86%, 87%, 88%, 89%, 90%, 91%, 92%, 93%, 94%, 95%, 96%, 97%, 98%, 99%, 99.9%, or 100% identical) to the amino acid sequence of SEQ ID NO: 2. Genes encoding a GAA polypeptide that may be used in conjunction with the compositions and methods described herein further include those that have one or more amino acid substitutions, such as those that have one or more conservative amino acid substitutions, with respect to the amino acid sequence set forth in SEQ ID NO: 2. For instance, GAA polypeptides that may be used in conjunction with the compositions and methods described herein include those that have 1, 2, 3, 4, 5, 6, 7, 8, 9, 10, 11, 12, 13, 14, 15, 16, 17, 18, 19, 20, 25, or more, conservative amino acid substitutions with respect to the amino acid sequence of SEQ ID NO: 2.

The transcription regulatory elements described herein can be operably linked to a transgene, such as GAA, that is deficient in lysosomal storage disease patients, such as those suffering from Pompe disease. Constructs containing a lysosomal enzyme under the transcriptional control of a regulatory element described herein can be incorporated into a vector (or other transfection agent described herein) and administered to a patient to treat a lysosomal storage disorder. Advantageously, the therapeutic agents (e.g., viral vectors) containing a transgene described herein may promote transcription of the gene encoding the deficient lysosomal enzyme (e.g., GAA) in those cells that are affected by the disease, such as muscle cells and cells of the central nervous system. Further, the therapeutic agents described herein impart the additional benefit of avoiding toxicity that may be associated with overexpression of GAA or administration of high quantities of a viral vector encoding the same.

### Transaminasemia and Hyperbilirubinemia

Transaminasemia is any condition in which liver transaminases are elevated, while hyperbilirubinemia is a condition in which there is an accumulation of bilirubin in the blood though serum corticosteroids appear to remain normal.

In some embodiments, the patient is monitored for the development of transaminasemia. In some embodiments, the patient is monitored for the development of hyperbilirubinemia. In some embodiments, the patient is monitored for the development of transaminasemia, hyperbilirubinemia, or one or more symptoms thereof. In some embodiments, the patient is monitored for the development of transaminasemia, hyperbilirubinemia, or one or more symptoms thereof by evaluating a parameter in a blood sample obtained from the patient, wherein a finding that the parameter is above a reference level identifies the patient as having transaminasemia, hyperbilirubinemia, or one or more symptoms thereof

In some embodiments, the patient is monitored for the development of hyperbilirubinemia and if the patient exhibits transaminasemia, hyperbilirubinemia, or one or more symptoms thereof, the patient is administered an anti-transaminitis agent.

In some embodiments, it is determined that the patient exhibits transaminasemia, hyperbilirubinemia, or one or more symptoms thereof and the patient is administered an anti-transaminitis agent.

In some embodiments, a patient is determined to exhibit transaminasemia or one or more symptoms thereof when the patient exhibits one or more parameters (e.g., aspartate aminotransferase (AST) level and/or alanine aminotransferase (ALT) level), as measured in a blood test (e.g., a liver function test (LFT)), that is greater than or less than the age-adjusted norm.

In some embodiments, a patient is determined to exhibit hyperbilirubinemia or one or more symptoms thereof when the patient exhibits a bilirubin level, as measured in a blood test (e.g., a bilirubin test), that is greater than the norm.

In some embodiments, the disclosure provides a method of treating transaminasemia in a human patient that has Pompe disease and who has been previously administered a viral vector including a transgene encoding GAA (e.g., the viral vector), the method including administering to the patient an anti-transaminitis agent.

In some embodiments, the disclosure provides a method of treating hyperbilirubinemia in a human patient that has Pompe disease and who has been previously administered a viral vector including a transgene encoding GAA (e.g., the viral vector), the method including administering to the patient an anti-transaminitis agent.

In some embodiments, the disclosure provides a method of preventing transaminasemia in a human patient that has Pompe disease and who has been previously administered a viral vector including a transgene encoding GAA (e.g., the viral vector), the method including administering to the patient an anti-transaminitis agent.

In some embodiments, the disclosure provides a method of preventing hyperbilirubinemia in a human patient that has Pompe disease and who has been previously administered a viral vector including a transgene encoding GAA (e.g., the viral vector), the method including administering to the patient an anti-transaminitis agent.

In some embodiments, the patient does not have a history of transaminasemia or hyperbilirubinemia. In some embodiments, the patient does not have a history of any underlying liver disease.

### Vectors for Delivery of Exogenous Nucleic Acids to Target Cells

### Viral Vectors for Nucleic Acid Delivery

Viral genomes provide a rich source of vectors that can be used for the efficient delivery of a gene of interest (e.g., a transgene encoding GAA) into the genome of a target cell (e.g., a mammalian cell, such as a human cell). Viral genomes are particularly useful vectors for gene delivery because the polynucleotides contained within such genomes are typically incorporated into the genome of a target cell by generalized or specialized transduction. These processes occur as part of the natural viral replication cycle, and do not require added proteins or reagents to induce gene integration. Examples of viral vectors include AAV, retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding antibody light and heavy chains or antibody fragments of the invention include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D-type viruses, HTLV-BLV group, lentivirus, spumavirus

(Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the disclosure of which is incorporated herein by reference as it pertains to viral vectors for use in gene therapy.

### AAV Vectors for Nucleic Acid Delivery

In some embodiments, nucleic acids of the compositions and methods described herein are incorporated into recombinant AAV (rAAV) vectors and/or virions to facilitate their introduction into a cell. rAAV vectors useful in the invention are recombinant nucleic acid constructs that include (1) a transgene to be expressed (e.g., a polynucleotide encoding a GAA protein) and (2) viral nucleic acids that facilitate integration and expression of the heterologous genes. The viral nucleic acids may include those sequences of AAV that are required in cis for replication and packaging (e.g., functional inverted terminal repeats (ITRs)) of the DNA into a virion. In typical applications, the transgene encodes GAA, which is useful for correcting a GAA mutation in patients suffering from glycogen storage disorders, such as Pompe disease. Such rAAV vectors may also contain marker or reporter genes. Useful rAAV vectors have one or more of the AAV wild type genes deleted in whole or in part but retain functional flanking ITR sequences. The AAV ITRs may be of any serotype (e.g., derived from serotype 2) suitable for a particular application. Methods for using rAAV vectors are described, for example, in Tal et al., J. Biomed. Sci. 7:279-291 (2000), and Monahan and Samulski, Gene Delivery 7:24-30 (2000), the disclosures of each of which are incorporated herein by reference as they pertain to AAV vectors for gene delivery.

The nucleic acids and vectors described herein can be incorporated into a rAAV virion to facilitate introduction of the nucleic acid or vector into a cell. The capsid proteins of AAV compose the exterior, non-nucleic acid portion of the virion and are encoded by the AAV cap gene. The cap gene encodes three viral coat proteins, VP1, VP2 and VP3, which are required for virion assembly. The construction of rAAV virions has been described, for example, in U.S. Pat. Nos. 5,173,414; 5,139,941; 5,863,541; 5,869,305; 6,057,152; and 6,376,237; as well as in Rabinowitz et al., J. Virol. 76:791-801 (2002) and Bowles et al., J. Virol. 77:423-432 (2003), the disclosures of each of which are incorporated herein by reference as they pertain to AAV vectors for gene delivery. rAAV virions useful in conjunction with the compositions and methods described herein include those derived from a variety of AAV serotypes including AAV 1, 2, 3, 4, 5, 6, 7, 8 and 9. For targeting muscle cells, rAAV virions that include at least one serotype 1 capsid protein may be particularly useful. rAAV virions that include at least one serotype 6 capsid protein may also be particularly useful, as serotype 6 capsid proteins are structurally similar to serotype 1 capsid proteins, and thus are expected to also result in high expression of GAA in muscle cells. rAAV serotype 9 has also been found to be an efficient transducer of muscle cells. Construction and use of AAV vectors and AAV proteins of different serotypes are described, for example, in Chao et al., Mol. Ther. 2:619-623 (2000); Davidson et al., Proc. Natl. Acad. Sci. USA 97:3428-3432 (2000); Xiao et al., J. Virol. 72:2224-2232 (1998); Halbert et al., J. Virol. 74:1524-1532 (2000); Halbert et al., J. Virol. 75:6615-6624 (2001); and Auricchio et al., Hum. Molec. Genet. 10:3075-3081 (2001), the disclosures of each of which are incorporated herein by reference as they pertain to AAV vectors for gene delivery.

Also useful in conjunction with the compositions and methods described herein are pseudotyped rAAV vectors. Pseudotyped vectors include AAV vectors of a given serotype (e.g., AAV9) pseudotyped with a capsid gene derived from a serotype other than the given serotype (e.g., AAV1, AAV2, AAV3, AAV4, AAV5, AAV6, AAV7, AAV8, etc.). For example, a representative pseudotyped vector is an AAV8 vector encoding a therapeutic protein pseudotyped with a capsid gene derived from AAV serotype 2. Techniques involving the construction and use of pseudotyped rAAV virions are known in the art and are described, for example, in Duan et al., J. Virol. 75:7662-7671 (2001); Halbert et al., J. Virol. 74:1524-1532 (2000); Zolotukhin et al., Methods, 28:158-167 (2002); and Auricchio et al., Hum. Molec. Genet., 10:3075-3081 (2001).

AAV virions that have mutations within the virion capsid may be used to infect particular cell types more effectively than non-mutated capsid virions. For example, suitable AAV mutants may have ligand insertion mutations for the facilitation of targeting AAV to specific cell types. The construction and characterization of AAV capsid mutants including insertion mutants, alanine screening mutants, and epitope tag mutants are described in Wu et al., J. Virol. 74:8635-45 (2000). Other rAAV virions that can be used in methods of the invention include those capsid hybrids that are generated by molecular breeding of viruses as well as by exon shuffling. See, e.g., Soong et al., Nat. Genet., 25:436-439 (2000) and Kolman and Stemmer, Nat. Biotechnol. 19:423-428 (2001).

### Methods for the Delivery of Exogenous Nucleic Acids to Target Cells

### Transfection Techniques

Techniques that can be used to introduce a transgene, such as a GAA transgene described herein, into a target cell are known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids (e.g., nucleic acids capable of expression in e.g., neurons, glial cells, or non-neural cells, such as colon and kidney cells). Electroporation of mammalian cells is described in detail, e.g., in Chu et al., Nucleic Acids Research 15:1311 (1987), the disclosure of which is incorporated herein by reference. A similar technique, NUCLEOFECTION™, utilizes an applied electric field to stimulate the uptake of exogenous polynucleotides into the nucleus of a eukaryotic cell. NUCLEOFECTION™ and protocols useful for performing this technique are described in detail, e.g., in Distler et al., Experimental Dermatology 14:315 (2005), as well as in US 2010/0317114, the disclosures of each of which are incorporated herein by reference.

An additional technique useful for the transfection of target cells is the squeeze-poration methodology. This technique induces the rapid mechanical deformation of cells to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., J. Vis. Exp. 81:e50980 (2013), the disclosure of which is incorporated herein by reference.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386, the disclosure of which is incorporated herein by reference. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers (described, e.g., in Dennig, Top Curr Chem. 228:227 (2003), the disclosure of which is incorporated herein by reference) polyethylenimine, and DEAE-dextran, the use of which as a transfection agent is described in detail, for example, in Gulick et al., Curr Protoc Mol Biol. 40:1:9.2:9.2.1 (1997), the disclosure of which is incorporated herein by reference.

Another useful tool for inducing the uptake of exogenous nucleic acids by target cells is laserfection, also called optical transfection, a technique that involves exposing a cell to electromagnetic radiation of a particular wavelength to gently permeabilize the cells and allow polynucleotides to penetrate the cell membrane. The bioactivity of this technique is like, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s). An example of this technique is described in Shalek et al., PNAS 107:25 1870 (2010), the disclosure of which is incorporated herein by reference.

MAGNETOFECTION™ can also be used to deliver nucleic acids to target cells. The principle of MAGNETOFECTION™ is to associate nucleic acids with cationic magnetic nanoparticles. The magnetic nanoparticles are made of iron oxide, which is fully biodegradable, and coated with specific cationic proprietary molecules varying upon the applications. Their association with the gene vectors (DNA, siRNA, viral vector, etc.) is achieved by salt-induced colloidal aggregation and electrostatic interaction. The magnetic particles are then concentrated on the target cells by the influence of an external magnetic field generated by magnets. This technique is described in detail in Scherer et al., Gene Ther. 9:102 (2002), the disclosure of which is incorporated herein by reference. Magnetic beads are another tool that can be used to transfect target cells in a mild and efficient manner, as this methodology utilizes an applied magnetic field to direct the uptake of nucleic acids. This technology is described in detail, for example, in US2010/0227406, the disclosure of which is incorporated herein by reference.

Another useful tool for inducing the uptake of exogenous nucleic acids by target cells is sonoporation, a technique that involves the use of sound (typically ultrasonic frequencies) for modifying the permeability of the cell plasma membrane permeabilize the cells and allow polynucleotides to penetrate the cell membrane. This technique is described in detail, e.g., in Rhodes et al., Methods Cell Biol. 82:309 (2007), the disclosure of which is incorporated herein by reference.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Incorporation of Target Genes by Gene Editing Techniques

In addition to the above, a variety of tools have been developed that can be used for the incorporation of a gene of interest into a target cell, such as a human cell. One such method that can be used for incorporating polynucleotides encoding target genes into target cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In some instances, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the target gene is first transcribed to an RNA product and then reverse-transcribed to DNA before incorporation in the mammalian cell genome. Exemplary transposon systems are the piggybac transposon (described in detail in, e.g., WO 2010/085699) and the sleeping beauty transposon (described in detail in, e.g., US 2005/0112764), the disclosures of each of which are incorporated herein by reference as they pertain to transposons for use in gene delivery to a cell of interest.

Another tool for the integration of target genes into the genome of a target cell is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas9 nuclease to this site. In this manner, highly site-specific cas9-mediated DNA cleavage can be engendered in a foreign polynucleotide because the interaction that brings cas9 within close proximity of the target DNA molecule is governed by RNA:DNA hybridization. As a result, one can design a CRISPR/Cas system to cleave any target DNA molecule of interest. This technique has been exploited to edit eukaryotic genomes (Hwang et al., Nature Biotechnology 31:227 (2013)) and can be used as an efficient means of site-specifically editing target cell genomes in order to cleave DNA prior to the incorporation of a gene encoding a target gene. The use of CRISPR/Cas to modulate gene expression has been described in, for example, U.S. Pat. No. 8,697,359, the disclosure of which is incorporated herein by reference as it pertains to the use of the CRISPR/Cas system for genome editing. Alternative methods for site-specifically cleaving genomic DNA prior to the incorporation of a gene of interest in a target cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al., Nat. Rev. Genet. 11:636 (2010); and in Joung et al., Nat. Rev. Mol. Cell Biol. 14:49 (2013), the disclosure of each of which are incorporated herein by reference as they pertain to compositions and methods for genome editing.

Additional genome editing techniques that can be used to incorporate polynucleotides encoding target genes into the genome of a target cell include the use of ARCUS™ meganucleases that can be rationally designed to site-specifically cleave genomic DNA. The use of these enzymes for the incorporation of genes encoding target genes into the genome of a mammalian cell is advantageous in view of the defined structure-activity relationships that have been established for such enzymes. Single chain meganucleases can be modified at certain amino acid positions to create nucleases that selectively cleave DNA at desired locations, enabling the site-specific incorporation of a target gene into the nuclear DNA of a target cell. These single-chain nucleases have been described extensively in, for example, U.S. Pat. Nos. 8,021,867 and 8,445,251, the disclosures of each of which are incorporated herein by reference as they pertain to compositions and methods for genome editing.

### Dosing Regimens

### Dosing Regimens Involving AAV-GAA Vectors

Using the compositions and methods of the disclosure, a patient having a glycogen storage disorder (e.g., Pompe disease) may be administered an AAV vector containing a transgene encoding GAA (e.g., the viral vector) in an amount of from about 1×10¹³ vg/kg to about 3×10¹⁴ vg/kg. Administration of the vector to the patient in such a quantity can achieve the beneficial effect of augmenting GAA expression in the patient, e.g., to within 50% or 200% of wild-type levels, without inducing toxic side effects.

In some embodiments, the AAV vector is administered to the patient in an amount of from about of from about 1×10¹³ vg/kg to about 3×10¹⁴ vg/kg (e.g., from about 1×10¹³ vg/kg to about 6×10¹³ vg/kg, from about 1×10¹³ vg/kg to about 5×10¹³ vg/kg, from about 1×10¹³ vg/kg to about 4×10¹³ vg/kg, from about 1×10¹³ vg/kg to about 3×10¹³ vg/kg, from about 2×10¹³ vg/kg to about 6×10¹³ vg/kg, from about 2×10¹³ vg/kg to about 5×10¹³ vg/kg, or from about 2×10¹³ vg/kg to about 4×10¹³ vg/kg). For example, the AAV vector may be administered to the patient in an amount of about 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, or 3×10¹⁴ vg/kg. Administration of the vector to the patient in such a quantity can achieve the beneficial effect of augmenting GAA expression in the patient, e.g., to within 50% or 200% of wild-type levels, without inducing toxic side effects.

For example, in some embodiments, the AAV vector is administered to the patient in an amount of from about 2×10¹³ vg/kg to about 2×10¹⁴ vg/kg, such as an in amount of about 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg. In some embodiments, the AAV vector is administered to the patient in an amount of from about 2×10¹³ vg/kg to about 7×10¹³ vg/kg, such as in an amount of from about 2×10¹³ vg/kg to about 4×10¹³ vg/kg (e.g., about 3×10¹³ vg/kg) or in an amount of from about 5×10¹³ vg/kg to about 7×10¹³ vg/kg (e.g., about 6×10¹³ vg/kg).

In some embodiments, the AAV vector is administered to the patient in an amount of from about 3×10¹³ vg/kg to about 2×10¹⁴ vg/kg, such as an in amount of about 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 4×10¹³ vg/kg to about 2×10¹⁴ vg/kg, such as an in amount of about 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 5×10¹³ vg/kg to about 2×10¹⁴ vg/kg, such as an in amount of about 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 6×10¹³ vg/kg to about 2×10¹⁴ vg/kg, such as an in amount of about 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 7×10¹³ vg/kg to about 2×10¹⁴ vg/kg, such as an in amount of about 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 8×10¹³ vg/kg to about 2×10¹⁴ vg/kg, such as an in amount of about 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 9×10¹³ vg/kg to about 2×10¹⁴ vg/kg, such as an in amount of 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of from about 1×10¹⁴ vg/kg to about 2×10¹⁴ vg/kg, such as an in amount 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg.

In some embodiments, the AAV vector is administered to the patient in an amount of 6×10¹³ vg/kg. In some embodiments, the AAV vector is administered to the patient in an amount of 7×10¹³ vg/kg. In some embodiments, the AAV vector is administered to the patient in an amount of 8×10¹³ vg/kg. In some embodiments, the AAV vector is administered to the patient in an amount of 9×10¹³ vg/kg. In some embodiments, the AAV vector is administered to the patient in an amount of 1×10¹⁴ vg/kg. In some embodiments, the AAV vector is administered to the patient in an amount of 1.1×10¹⁴ vg/kg. In some embodiments, the AAV vector is administered to the patient in an amount of 1.2×10¹⁴ vg/kg. In some embodiments, the AAV vector is administered to the patient in an amount of 1.3×10¹⁴ vg/kg. In some embodiments, the AAV vector is administered to the patient in an amount of 1.4×10¹⁴ vg/kg. In some embodiments, the AAV vector is administered to the patient in an amount of 1.5×10¹⁴ vg/kg. In some embodiments, the AAV vector is administered to the patient in an amount of 1.6×10¹⁴ vg/kg. In some embodiments, the AAV vector is administered to the patient in an amount of 1.7×10¹⁴ vg/kg. In some embodiments, the AAV vector is administered to the patient in an amount of 1.8×10¹⁴ vg/kg. In some embodiments, the AAV vector is administered to the patient in an amount of 1.9×10¹⁴ vg/kg. In some embodiments, the AAV vector is administered to the patient in an amount of 2×10¹⁴ vg/kg.

In some embodiments, the AAV vector is administered to the patient in a single dose including the amount (e.g., from about 1×10¹³ vg/kg to about 3×10¹⁴ vg/kg).

In some embodiments, the AAV vector is administered to the patient in two or more (e.g., two, three, four, five, six, seven, eight, nine, or ten) doses that, together, include the amount (e.g., from about 1×10¹³ vg/kg to about 3×10¹⁴ vg/kg).

In some embodiments, the AAV vector is administered to the patient in two or more (e.g., two, three, four, five, six, seven, eight, nine, or ten) doses that each, individually, include the amount (e.g., from about 1×10³ vg/kg to about 3×10¹⁴ vg/kg).

In some embodiments, the two or more (e.g., two, three, four, five, six, seven, eight, nine, or ten) doses are separated from one another by one year or more (e.g., one year, one year and one day, one year and one month, one year and six months, two years, three years, four years, or five years).

In some embodiments, the two or more (e.g., two, three, four, five, six, seven, eight, nine, or ten) doses are administered to the patient within about 12 months (e.g., about 12 months, about 11 months, about 10 months, about 9 months, about 8 months, about 7 months, about 6 months, about 5 months, about 4 months, about 3 months, about 2 months, or about 1 month) of one another.

### Pharmaceutical Compositions and Routes of Administration

The gene therapy agents described herein may contain a transgene, such as a transgene encoding GAA and may be incorporated into a vehicle for administration into a patient, such as a human patient suffering from a glycogen storage disorder (for example, Pompe disease). Pharmaceutical compositions containing vectors, such as viral vectors, that contain the transcription regulatory elements (e.g., a MCK promoter) described herein operably linked to a therapeutic transgene can be prepared using methods known in the art. For example, such compositions can be prepared using, e.g., physiologically acceptable carriers, excipients or stabilizers (Remington's Pharmaceutical Sciences 16th edition, Osol, A. Ed. (1980); incorporated herein by reference), and in a desired form, e.g., in the form of lyophilized formulations or aqueous solutions.

Viral vectors, such as AAV vectors and others described herein, containing the transcription regulatory element operably linked to a therapeutic transgene may be administered to a patient (e.g., a human patient) by a variety of routes of administration. The route of administration may vary, for example, with the onset and severity of disease, and may include, e.g., intradermal, transdermal, parenteral, intravenous, intramuscular, intranasal, subcutaneous, percutaneous, intratracheal, intraperitoneal, intraarterial, intravascular, inhalation, perfusion, lavage, and oral administration. Intravascular administration includes delivery into the vasculature of a patient. In some embodiments, the administration is into a vessel considered to be a vein (intravenous), and in some administration, the administration is into a vessel considered to be an artery (intraarterial). Veins include, but are not limited to, the internal jugular vein, a peripheral vein, a coronary vein, a hepatic vein, the portal vein, great saphenous vein, the pulmonary vein, superior vena cava, inferior vena cava, a gastric vein, a splenic vein, inferior mesenteric vein, superior mesenteric vein, cephalic vein, and/or femoral vein. Arteries include, but are not limited to, coronary artery, pulmonary artery, brachial artery, internal carotid artery, aortic arch, femoral artery, peripheral artery, and/or ciliary artery. It is contemplated that delivery may be through or to an arteriole or capillary.

Mixtures of the nucleic acids and viral vectors described herein may be prepared in water suitably mixed with one or more excipients, carriers, or diluents. Dispersions may also be prepared in glycerol, liquid polyethylene glycols, and mixtures thereof and in oils. Under ordinary conditions of storage and use, these preparations may contain a preservative to prevent the growth of microorganisms. The pharmaceutical forms suitable for injectable use include sterile aqueous solutions or dispersions and sterile powders for the extemporaneous preparation of sterile injectable solutions or dispersions (described in U.S. Pat. No. 5,466,468, the disclosure of which is incorporated herein by reference). In any case the formulation may be sterile and may be fluid to the extent that easy syringability exists. Formulations may be stable under the conditions of manufacture and storage and may be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, using a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For example, a solution containing a pharmaceutical composition described herein may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion. Some variation in dosage will necessarily occur depending on the condition of the subject being treated. The person responsible for administration will, in any event, determine the appropriate dose for the individual subject. Moreover, for human administration, preparations may meet sterility, pyrogenicity, general safety, and purity standards as required by FDA Office of Biologics standards.

### Combination Therapies

An AAV vector containing a transgene encoding GAA (e.g., the viral vector) described herein can be administered in combination with a one or more additional therapeutic procedures and/or agents (e.g., an anti-transaminitis agent) for the treatment of a glycogen storage disorder (e.g., Pompe disease).

### Additional Therapeutic Agent

In some embodiments, the one or more additional therapeutic agents is an anti-transaminitis agent (e.g., a corticosteroid, a bile acid, a farnesoid X receptor (FXR) ligand, a fibroblast growth factor 19 (FGF-19) mimetic, a Takeda-G-protein-receptor-5 (TGR5) agonist, a peroxisome proliferator-activated receptor (PPAR) agonist, a PPAR-alpha agonist, a PPAR-delta agonist, a dual PPAR-alpha and PPAR-delta agonist, an apical sodium-dependent corticosteroid transporter (ASBT) inhibitor, an immunomodulatory drug, an antifibrotic therapy, and a nicotinamide adenine dinucleotide phosphate oxidase (NOX) inhibitor) or a combination thereof.

In some embodiments, the anti-transaminitis agent is administered to the patient in one or more (e.g., one, two, three, four, five, six, seven, eight, nine, ten, 15, 20, 30, 40, fifty, sixty, and seventy) doses that commence within about 48 weeks before or after (e.g., about 48 weeks before or after, about 36 weeks before or after, about 24 weeks before or after, about 12 weeks before or after, about 10 weeks before or after, about 8 weeks before or after, or about 4 weeks before or after) administration of the viral vector to the patient.

In some embodiments, the anti-transaminitis agent is administered to the patient in one or more (e.g., one, two, three, four, five, six, seven, eight, nine, ten, 15, 20, 30, 40, fifty, sixty, and seventy) doses that commence within about five weeks before or after (e.g., about five weeks before or after, about four weeks before or after, about three weeks before or after, about two weeks before or after, or about one week before or after) administration of the viral vector to the patient.

In some embodiments, the anti-transaminitis agent is administered to the patient in one or more (e.g., one, two, three, four, five, six, seven, eight, nine, ten, 15, 20, 30, 40, fifty, sixty, and seventy) doses that commence within about one week before or after (e.g., about one week before or after, about six days before or after, about five days before or after, about four days before or after, about three days before or after, about two days before or after, or about one day before or after) administration of the viral vector to the patient.

In some embodiments, the anti-transaminitis agent is administered to the patient in one or more (e.g., one, two, three, four, five, six, seven, eight, nine, ten, 15, 20, 30, 40, fifty, sixty, and seventy) doses that commence on the same day (e.g., 24^{th} hour, on the 23^{rd} hour, on the 22^{nd} hour, on the 21^{st} hour, on the 20^{th} hour, on the 19^{th} hour, on the 18^{th} hour, on the 17^{th} hour, on the 16^{th} hour, on the 15^{th} hour, on the 14^{th} hour, on the 13^{th} hour, on the 12^{th} hour, on the 11^{th} hour, on the 10^{th} hour, on the 9^{th} hour, on the 8^{th} hour, on the 7^{th} hour, on the 6^{th} hour, on the 5^{th} hour, on the 4^{th} hour, on the 3^{rd} hour, on the 2^{nd} hour, on the 1^{st} hour, on the 60^{th} minute, on the 59^{th} minute, on the 58^{th} minute, on the 57^{th} minute, on the 56^{th} minute, on the 55^{th} minute, on the 50^{th} minute, on the 40^{th} minute, on the 30^{th} minute, on the 20^{th} minute, on the 10^{th} minute, or on the same minute) of administration of the viral vector to the patient.

Using the compositions and methods of the disclosure, a patient having a glycogen storage disorder (e.g., Pompe disease) may be administered an AAV vector containing a transgene encoding GAA and an anti-transaminitis agent.

In some embodiments, a patient is administered an anti-transaminitis agent.

In some embodiments, a patient is administered an anti-transaminasemia when a patient is monitored for transaminasemia, hyperbilirubinemia, or one or more symptoms thereof and it is determined that the patient exhibits transaminasemia or hyperbilirubinemia or one or more symptoms thereof.

In some embodiments, a patient is administered an anti-transaminasemia when it is determined that the patient exhibits transaminasemia or hyperbilirubinemia or one or more symptoms thereof.

In some embodiments, the anti-transaminitis agent is selected from the list including a corticosteroid, a farnesoid X receptor (FXR) ligand, a fibroblast growth factor 19 (FGF-19) mimetic, a Takeda-G-protein-receptor-5 (TGR5) agonist, a peroxisome proliferator-activated receptor (PPAR) agonist, a PPAR-alpha agonist, a PPAR-delta agonist, a dual PPAR-alpha and PPAR-delta agonist, an apical sodium-dependent corticosteroid transporter (ASBT) inhibitor, an immunomodulatory drug, an antifibrotic therapy, and a nicotinamide adenine dinucleotide phosphate oxidase (NOX) inhibitor.

In some embodiments, the anti-transaminitis agent is a corticosteroid. In some embodiments, the corticosteroid is cortisone, prednisone, prednisolone, methylprednisolone, dexamethasone, betamethasone, or hydrocortisone. In some embodiments, the corticosteroid is prednisolone.

In some embodiments, the anti-transaminitis agent is a bile acid. In some embodiments, the bile acid is ursodeoxycholic acid or a derivative thereof or nor-ursodeoxycholic acid. In some embodiments, the bile acid is ursodiol.

In some embodiments, the anti-transaminitis agent is an FXR ligand. In some embodiments, the FXR ligand is obeticholic acid, cilofexor, tropifexor, tretinoin, or EDP-305.

In some embodiments, the one or more anti-transaminitis agent is an FGF-19 mimetic. In some embodiment, the FGF-19 mimetic is aldafermin.

In some embodiments, the anti-transaminitis agent is a TGR5 agonist. In some embodiments, the TGR5 agonist is INT-777 or INT-767.

In some embodiments, the anti-transaminitis agent is a PPAR agonist. In some embodiments, the PPAR agonist is bezafibrate, seladelpar, or elafibrinor.

In some embodiments, the anti-transaminitis agent is a PPAR-alpha agonist. In some embodiments, the PPAR-alpha agonist is fenofibrate.

In some embodiments, the anti-transaminitis agent is a PPAR-delta agonist. In some embodiments, the PPAR-delta agonist is seladelpar.

In some embodiments, the anti-transaminitis agent is a dual PPAR-alpha and PPAR-delta agonist. In some embodiments, the dual PPAR-alpha-delta agonist is elafibranor.

In some embodiments, the one or more anti-transaminitis agent is an ASBT inhibitor. In some embodiments, the ASBT inhibitor is odevixibat, maralixibat, or linerixibat.

In some embodiments, the anti-transaminitis agent is an immunomodulatory drug. In some embodiments, the immunomodulatory drug is rituximab, abatacept, ustekinumab, infliximab, baricitinib, or FFP104.

In some embodiments, the anti-transaminitis agent is an antifibrotic therapy. In some embodiments, the antifibrotic therapy is a vitamin D receptor (VDR) agonist or simtuzumab.

In some embodiments, the anti-transaminitis agent is a NOX inhibitor. In some embodiments, the NOX inhibitor is setanaxib.

In some embodiments, a therapeutically effective amount of a viral vector including a transgene encoding GAA (e.g., the viral vector) and an anti-transaminitis agent are administered to a patient in need thereof. In some embodiments, a therapeutically effective amount of the viral vector and an anti-transaminitis agent are administered to a patient in need thereof. In some embodiments, a therapeutically effective amount of the viral vector and an anti-transaminitis agent are administered to a patient in need thereof, wherein the anti-transaminitis agent is a corticosteroid. In some embodiments, a therapeutically effective amount of the viral vector and an anti-transaminitis agent are administered to a patient in need thereof, wherein the anti-transaminitis agent is prednisolone. In some embodiments, a therapeutically effective amount of the viral vector and prednisolone are administered to a patient in need thereof.

### I. Corticosteroid

Using the methods described herein, a corticosteroid can be administered to the subject. In some embodiments, the corticosteroid is cortisone, prednisone, prednisolone, methylprednisolone, dexamethasone, betamethasone, or hydrocortisone. In some embodiments, the corticosteroid is prednisolone.

Ia. Cortisone

Using the methods described herein, cortisone can be administered to the subject. Cortisone has the chemical structure depicted below.

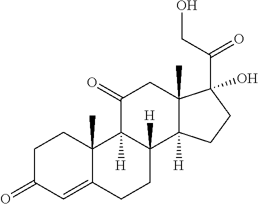

Ib. Prednisone

Using the methods described herein, prednisone can be administered to the subject. Prednisone has the chemical structure depicted below.

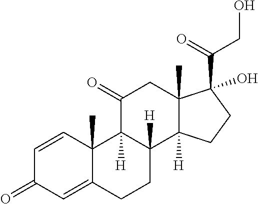

Ic. Prednisolone

Using the methods described herein, prednisolone can be administered to the subject. Prednisone has the chemical structure depicted below.

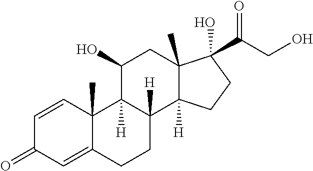

Ici. Dosing Regimens Involving Prednisolone

Prednisolone described herein may be administered in an amount of from about 0.1 mg/kg/dose to about 2 mg/kg/dose (e.g., about e.g., 0.2 mg/kg/dose to about 1.9 mg/kg/dose, 0.3 mg/kg/dose to about 1.8 mg/kg/dose, 0.4 mg/kg/dose to about 1.7 mg/kg/dose, 0.5 mg/kg/dose to about 1.6 mg/kg/dose, 1 mg/kg/dose to about 1.5 mg/kg/dose). For example, in some embodiments, prednisolone is administered to the patient in an amount of about 0.2 mg/kg/dose to about 1.9 mg/kg/dose. In some embodiments, prednisolone is administered to the patient in an amount of about 0.3 mg/kg/dose to about 1.8 mg/kg/dose. In some embodiments, prednisolone is administered to the patient in an amount of about 0.4 mg/kg/dose to about 1.7 mg/kg/dose. In some embodiments, prednisolone is administered to the patient in an amount of about 0.5 mg/kg/dose to about 1.6 mg/kg/dose. In some embodiments, prednisolone is administered to the patient in an amount of about 1 mg/kg/dose to about 1.5 mg/kg/dose.

In some embodiments, prednisolone is administered to the patient in an amount of about 0.5 mg/kg/dose. In some embodiments, prednisolone is administered to the patient in an amount of about 1 mg/kg/dose. In some embodiments, prednisolone is administered to the patient in an amount of about 2 mg/kg/dose.

For example, in some embodiments, prednisolone is administered to the patient in an amount of from about 1 mg to about 120 mg (e.g. about 2 mg to about 119 mg, 3 mg to about 118 mg, 4 mg to about 117 mg, 5 mg to about 116 mg, 10 mg to about 115 mg, 20 mg to about 110 mg, 30 mg to about 100 mg, 40 mg to about 90 mg, 50 mg to about 80 mg, or 60 mg to about 70 mg). In some embodiments, prednisolone is administered to the patient in an amount of from about 2 mg to about 119 mg. In some embodiments, prednisolone is administered to the patient in an amount of from about 3 mg to about 118 mg. In some embodiments, prednisolone is administered to the patient in an amount of from about 4 mg to about 117 mg. In some embodiments, prednisolone is administered to the patient in an amount of from about 5 mg to about 116 mg. In some embodiments, prednisolone is administered to the patient in an amount of from about 10 mg to about 115 mg. In some embodiments, prednisolone is administered to the patient in an amount of from about 20 mg to about 110 mg. In some embodiments, prednisolone is administered to the patient in an amount of from about 30 mg to about 100 mg. In some embodiments, prednisolone is administered to the patient in an amount of from about 40 mg to about 90 mg. In some embodiments, prednisolone is administered to the patient in an amount of from about 50 mg to about 80 mg. In some embodiments, prednisolone is administered to the patient in an amount of from about 60 mg to about 70 mg.

In some embodiments, prednisolone is administered to the patient in an amount of about 5 mg. In some embodiments, prednisolone is administered to the patient in an amount of about 10 mg. In some embodiments, prednisolone is administered to the patient in an amount of about 15 mg. In some embodiments, prednisolone is administered to the patient in an amount of about 30 mg. In some embodiments, prednisolone is administered to the patient in an amount of about 60 mg. In some embodiments, prednisolone is administered to the patient in an amount of about 120 mg.

In some embodiments, prednisolone is administered to the patient in a single dose.

In some embodiments, prednisolone is administered to the patient in a plurality of doses.

In some embodiments, the prednisolone is administered to the patient in one or more doses per day (one dose per day, two doses per day, three doses per day, four doses per day, five doses per day, six doses per day, seven doses per day, eight doses per day, nine doses per day, and ten doses per day), week (one dose per week, two doses per week, three doses per week, four doses per week, five doses per week, six doses per week, seven doses per week, eight doses per week, nine doses per week, and ten doses per week, 11 doses per week, 12 doses per week, 13 doses per week, and 14 doses per week), or month (one dose per month, two doses per month, three doses per month, four doses per month, five doses per month, six doses per month, seven doses per month, eight doses per month, nine doses per month, and ten doses per month, 11 doses per month, 12 doses per month, 13 doses per month, 14 doses per month, 15 doses per month, 16 doses per month, 17 doses per month, 18 doses per month, 19 doses per month, 20 doses per month, 21 doses per month, 22 doses per month, 23 doses per month, 24 doses per month, 25 doses per month, 26 doses per month, 27 doses per month, 28 doses per month, 29 doses per month, and 30 doses per month).

For example, in some embodiments, prednisolone is administered to the patient in one or more doses per day, such as in one dose per day, two doses per day, three doses per day, four doses per day, five doses per day, six doses per day, seven doses per day, eight doses per day, nine doses per day, or ten doses per day.

In some embodiments, the two or more doses of prednisolone that, together, total the specified amount are separated from one another, for example, by an hour or more. In some embodiments, the two or more doses are administered to the patient within about 24 hours of one another (e.g., within about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, or 24 hours of one another).

In some embodiments, prednisolone is administered to the patient in one dose per day, two doses per day, three doses per day, four doses per day, or five doses per day.

In some embodiments, prednisolone is administered to the patient in one dose per day.

In some embodiments, prednisolone is administered in an amount of from about 1 mg/day to about 120 mg/day (e.g., in an amount of from about 2 mg/day to about 119 mg/day, 3 mg/day to about 118 mg/day, 4 mg/day to about 117 mg/day, 5 mg/day to about 116 mg/day, 10 mg/day to about 115 mg/day, 20 mg/day to about 110 mg/day, 30 mg/day to about 100 mg/day, 40 mg/day to about 90 mg/day, 50 mg/day to about 80 mg/day, or 60 mg/day to about 70 mg/day). For example, in some embodiments, prednisolone is administered to the patient in an amount of about 2 mg/day to about 119 mg/day. In some embodiments, prednisolone is administered to the patient in an amount of about 3 mg/day to about 118 mg/day. In some embodiments, prednisolone is administered to the patient in an amount of about 4 mg/day to about 117 mg/day. In some embodiments, prednisolone is administered to the patient in an amount of about 5 mg/day to about 116 mg/day. In some embodiments, prednisolone is administered to the patient in an amount of about 10 mg/day to about 115 mg/day. In some embodiments, prednisolone is administered to the patient in an amount of about 20 mg/day to about 110 mg/day. In some embodiments, prednisolone is administered to the patient in an amount of about 30 mg/day to about 100 mg/day. In some embodiments, prednisolone is administered to the patient in an amount of about 40 mg/day to about 90 mg/day. In some embodiments, prednisolone is administered to the patient in an amount of about 50 mg/day to about 80 mg/day. In some embodiments, prednisolone is administered to the patient in an amount of about 60 mg/day to about 70 mg/day.

For example, in some embodiments, prednisolone is administered to the patient in an amount of about 1 mg/day. In some embodiments, prednisolone is administered to the patient in an amount of about 2 mg/day. In some embodiments, prednisolone is administered to the patient in an amount of about 3 mg/day. In some embodiments, prednisolone is administered to the patient in an amount of about 4 mg/day. In some embodiments, prednisolone is administered to the patient in an amount of about 5 mg/day. In some embodiments, prednisolone is administered to the patient in an amount of about 10 mg/day. In some embodiments, prednisolone is administered to the patient in an amount of about 20 mg/day. In some embodiments, prednisolone is administered to the patient in an amount of about 30 mg/day. In some embodiments, prednisolone is administered to the patient in an amount of about 60 mg/day. In some embodiments, prednisolone is administered to the patient in an amount of about 120 mg/day.

In some embodiments, the corticosteroid is administered to the patient in an amount of about 30 mg/day. In some embodiments, the corticosteroid is administered to the patient in an amount of about 60 mg/day. In some embodiments, the corticosteroid is administered to the patient in an amount of about 120 mg/day.

In prednisolone is administered to the patient in a dose that tapers down. For example, in some embodiment, the tapering down occurs over days. In some embodiments, the tapering down occurs over weeks.

In some embodiments, prednisolone is administered to the patient in one or more doses per week, such as in one dose per week, two doses per week, three doses per week, four doses per week, five doses per week, ten doses per week, 15 doses per week, 20 doses per week, 30 doses per week, fifty doses per week, sixty doses per week, and seventy doses per week.

In some embodiments, prednisolone is administered to the patient in one or more doses per month, such as in one dose per month, two doses per month, three doses per month, four doses per month, five doses per month, ten doses per month, 15 doses per month, 20 doses per month, 30 doses per month, fifty doses per month, sixty doses per month, seventy doses per month, eighty doses per month, ninety doses per month, one hundred doses per month, two hundred doses per month, and three hundred doses per month.

In some embodiments, prednisolone is administered to the patient by way of a unit dosage form including 5 mg of the prednisolone.

In some embodiments, prednisolone is administered to the patient by way of a unit dosage form including 10 mg of the prednisolone.

In some embodiments, prednisolone is administered to the patient by way of a unit dosage form including 15 mg of the prednisolone.

In some embodiments, prednisolone is administered to the patient by way of a unit dosage form including 30 mg of the prednisolone.

Id. Methylprednisolone

Using the methods described herein, methylprednisolone can be administered to the subject. Prednisone has the chemical structure depicted below.

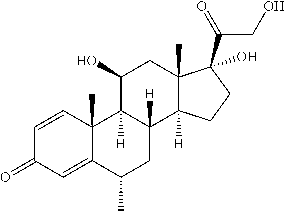

Ie. Dexamethasone

Using the methods described herein, dexamethasone can be administered to the subject. Prednisone has the chemical structure depicted below.

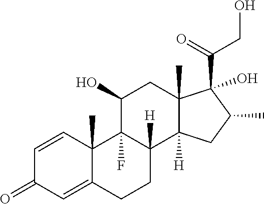

If. Hydrocortisone

Using the methods described herein, hydrocortisone can be administered to the subject. Prednisone has the chemical structure depicted below.

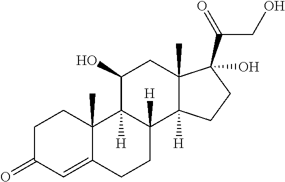

### II. Bile Acid

Using the methods described herein, a bile acid can be administered to the subject. In some embodiments, the bile acid is ursodeoxycholic acid or a derivative thereof or nor-ursodeoxycholic acid. In some embodiments, the bile acid is ursodiol.

Ursodiol and other known variants have the genus structure depicted below:

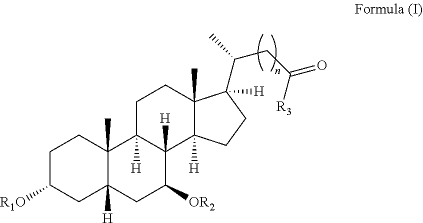

wherein each of R₁ and R₂ is, independently, hydrogen, optionally substituted alkyl, optionally substituted alkenyl, optionally substituted alkynyl, optionally substituted cycloalkyl, optionally substituted heterocyclyl, optionally substituted aryl, or optionally substituted heteroaryl;
R₃ is OR₄, NHR₄, or SR₄;
R₄ is hydrogen, optionally substituted alkyl, optionally substituted alkenyl, optionally substituted alkynyl, optionally substituted cycloalkyl, optionally substituted heterocyclyl, optionally substituted aryl, or optionally substituted heteroaryl; and
n is an integer from 0 to 4,
or a pharmaceutically acceptable salt thereof.

Such compounds are described in, e.g., U.S. Pat. No. 4,828,763, the disclosure of which is incorporated herein by reference.

Ia. Nor-Ursodeoxycholic Acid

Using the methods described herein, nor-Ursodeoxycholic acid can be administered to the subject.

Nor-Ursodeoxycholic acid is the INN for the compound with the chemical structure depicted below.

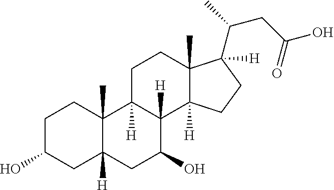

### III. FXR Ligand

Using the methods described herein, an FXR ligand can be administered to the subject. In some embodiments, the FXR ligand is obeticholic acid, cilofexor, tropifexor, tretinoin, or EDP-305.

IIIa. Obeticholic Acid

Using the methods described herein, obeticholic acid can be administered to the subject. Obeticholic acid is the INN for the compound also known by the code name of INT-747. Obeticholic acid has the chemical structure depicted below.

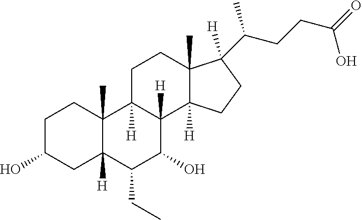

IIIb. Cilofexor

Using the methods described herein, cilofexor can be administered to the subject. Cilofexor is the INN for the compound also known by the code name of GS-9674. Cilofexor has the chemical structure depicted below.

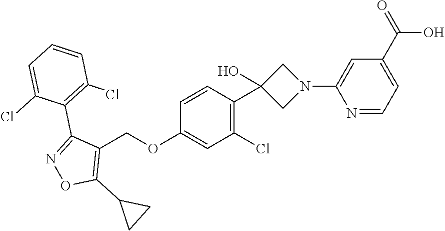 [SMILES: O=C(O)c1ccnc(N2CC(O)(c3ccc(OCc4c(-c5c(Cl)cccc5Cl)noc4C4CC4)cc3Cl)C2)c1]

IIIc. Tropifexor

Using the methods described herein, tropifexor can be administered to the subject. Tropifexor is the INN for the compound also known by the code name of LJN452. Tropifexor has the chemical structure depicted below.

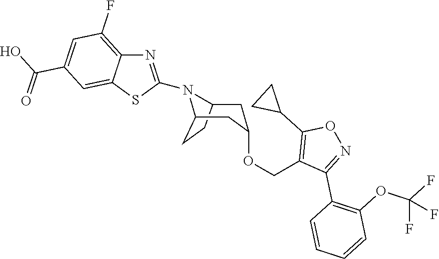 [SMILES: O=C(O)c1cc(F)c2nc(N3C4CCC3CC(OCc3c(-c5ccccc5OC(F)(F)F)noc3C3CC3)C4)sc2c1]

IIId. Tretinoin

Using the methods described herein, tretinoin can be administered to the subject. Tretinoin is the INN for the compound also known by the code name of 302-79-4. Retinoin has the chemical structure depicted below.

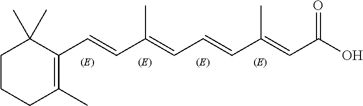 [SMILES: CC1=C(/C=C/C(C)=C/C=C/C(C)=C/C(=O)O)C(C)(C)CCC1]

IIIe. EDP-305

Using the methods described herein, EDP-305 can be administered to the subject. EDP-305 is the code name of compound with the chemical structure depicted below.

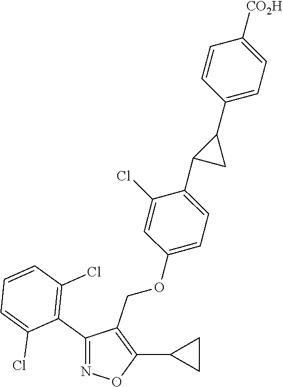 [SMILES: O=C(O)c1ccc(C2CC2c2ccc(OCc3c(-c4c(Cl)cccc4Cl)noc3C3CC3)cc2Cl)cc1]

### IV. Fibroblast Growth Factor 19 (FGF-19) Mimetic

Using the methods described herein, an FGF-19 mimetic can be administered to the subject. In some embodiment, the FGF-19 mimetic is aldafermin.

IVa. Aldafermin

Using the methods described herein, aldafermin can be administered to the subject. Aldafermin is the INN for the compound also known by the code name of NGM282 and the chemical formula of C₉₄—H₁₄₇₂N₂₆₆O₂₇₉S₁₁.

### V. Takeda-G-Protein-Receptor-5 (TGR5) Agonist

Using the methods described herein, a TGR5 agonist can be administered to the subject. In some embodiments, the TGR5 agonist is INT-777 or INT-767.

Va. INT-777

Using the methods described herein, INT-777 can be administered to the subject. INT-777 is the code name of compound also known by the name of S-EMCA.

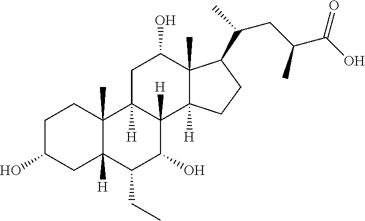

Vb. INT-767

Using the methods described herein, INT-767 can be administered to the subject. INT-767 is the code name of compound with the chemical structure depicted below.

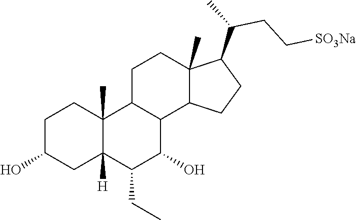

### VI. Peroxisome Proliferator-Activated Receptor (PPAR) Agonist

Using the methods described herein, a PPAR agonist can be administered to the subject. In some embodiments, the PPAR agonist is bezafibrate, seladelpar, or elafibrinor.

VIa. Bezafibrate

Using the methods described herein, Bezafibrate can be administered to the subject. Bezafibrate is the INN for the compound also known by the code name of C10AB02. Bezafibrate has the chemical structure depicted below.

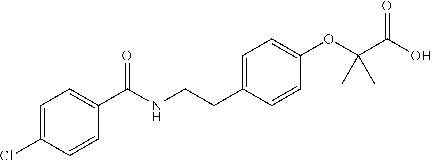 [SMILES: CC(C)(Oc1ccc(CCNC(=O)c2ccc(Cl)cc2)cc1)C(=O)O]

VIb. Seladelpar

Using the methods described herein, seladelpar can be administered to the subject. Seladelpar is the INN for the compound also known by the code name of MBX-8025. Seladelpar has the chemical structure depicted below.

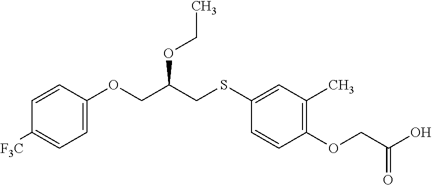 [SMILES: CCO[C@H](COc1ccc(C(F)(F)F)cc1)CSc1ccc(OCC(=O)O)c(C)c1]

VIc. Elafibrinor

Using the methods described herein, elafibrinor can be administered to the subject. Elafibrinor is the INN for the compound also known by the code name of GFT505. Elafibrinor has the chemical structure depicted below.

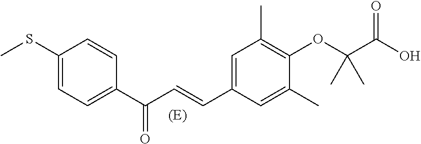 [SMILES: CSc1ccc(C(=O)/C=C/c2cc(C)c(OC(C)(C)C(=O)O)c(C)c2)cc1]

### VII. PPAR-Alpha Agonist

Using the methods described herein, a PPAR-alpha agonist can be administered to the subject. In some embodiments, the PPAR-alpha agonist is fenofibrate.

VIIa. Fenofibrate

Using the methods described herein, fenofibrate can be administered to the subject. Fenofibrate is the INN for the compound with the chemical structure depicted below.

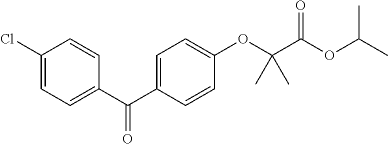 [SMILES: CC(C)OC(=O)C(C)(C)Oc1ccc(C(=O)c2ccc(Cl)cc2)cc1]

### VIII. PPAR-Delta Agonist

Using the methods described herein, a PPAR-delta agonist can be administered to the subject. In some embodiments, the PPAR-delta agonist is seladelpar.

VIIIa. Seladelpar

Using the methods described herein, seladelpar can be administered to the subject. Seladelpar is the INN for the compound also known by the code name of MBX-8025. Seladelpar has the chemical structure depicted below.

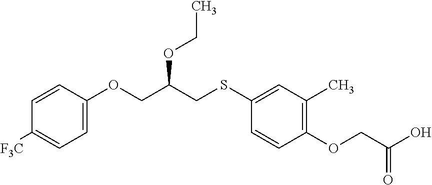 [SMILES: CCO[C@H](COc1ccc(C(F)(F)F)cc1)CSc1ccc(OCC(=O)O)c(C)c1]

### IX. Dual PPAR-Alpha and PPAR-Delta Agonist

Using the methods described herein, a dual PPAR-alpha and PPAR-delta agonist can be administered to the subject. In some embodiments, the dual PPAR-alpha-delta agonist is elafibranor.

IXa. Elafibranor

Using the methods described herein, elafibrinor can be administered to the subject. Elafibrinor is the INN for the compound also known by the code name of GFT505. Elafibrinor has the chemical structure depicted below.

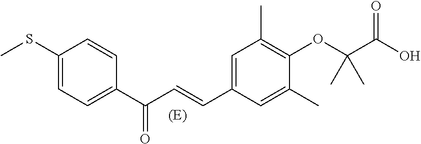 [SMILES: CSc1ccc(C(=O)/C=C/c2cc(C)c(OC(C)(C)C(=O)O)c(C)c2)cc1]

### X. Apical Sodium-Dependent Bile Acid Transporter (ASBT) Inhibitor

Using the methods described herein, an ASBT inhibitor can be administered to the subject. In some embodiments, the ASBT inhibitor is odevixibat, maralixibat, or linerixibat.

Xa. Odevixibat

Using the methods described herein, odevixibat can be administered to the subject. Odevixibat is the INN for the compound also known by the code name of A4250. Odevixibat has the chemical structure depicted below.

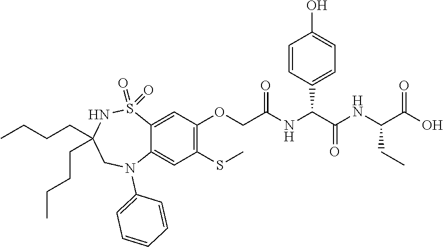 [SMILES: CCCCC1(CCCC)CN(c2ccccc2)c2cc(SC)c(OCC(=O)N[C@@H](C(=O)N[C@@H](CC)C(=O)O)c3ccc(O)cc3)cc2S(=O)(=O)N1]

Xb. Maralixibat

Using the methods described herein, maralixibat can be administered to the subject. Maralixibat is the INN for the compound with the chemical structure depicted below.

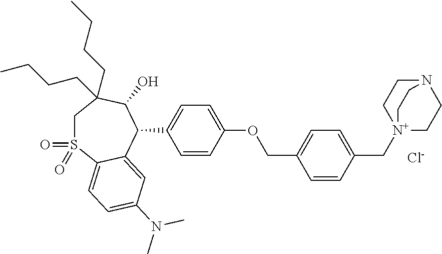 [SMILES: CCCCC1(CCCC)CS(=O)(=O)c2ccc(N(C)C)cc2[C@@H](c2ccc(OCc3ccc(C[N+]45CCN(CC4)CC5)cc3)cc2)[C@H]1O ; [Cl-]]

Xc. Linerixibat

Using the methods described herein, linerixibat can be administered to the subject. Linerixibat is the INN for the compound with the chemical structure depicted below.

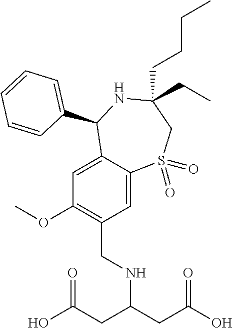 [SMILES: CCCC[C@]1(CC)CS(=O)(=O)c2cc(CNC(CC(=O)O)CC(=O)O)c(OC)cc2[C@@H](c2ccccc2)N1]

### XI. Immunomodulatory Drug

Using the methods described herein, an immunomodulatory drug can be administered to the subject. In some embodiments, the immunomodulatory drug is rituximab, abatacept, ustekinumab, infliximab, baricitinib, or FFP104.

Xca. Rituximab

Using the methods described herein, rituximab can be administered to the subject. Rituximab is the INN for the antibody with the chemical formula C₆₄₁₆—H₉₈₇₄—N₁₆₈₈—O₁₉₈₇—S₄₄.

XIb. Abatacept

Using the methods described herein, abatacept can be administered to the subject. Abatacept is the INN for the antibody with the chemical formula C₃₄₉₈H₄₈N₉₂₂O₁₀₉₀S₃₂.

XIc. Ustekinumab

Using the methods described herein, ustekinumab can be administered to the subject. Ustekinumab is the INN for the antibody with the chemical formula C₆₄₈₂H₁₀₀₀₄N₁₇₁₂O₂₀₁₆S₄₆.

XId. Infliximab

Using the methods described herein, infliximab can be administered to the subject. Infliximab is the INN for the antibody with the chemical formula C₆₄₂₈H₉₉₁₂N₁₆₉₄O₁₉₈₇S₄₆.

XIe. Baricitinib

Using the methods described herein, baricitinib can be administered to the subject. Baricitinib is the INN for the compound with the chemical structure depicted below.

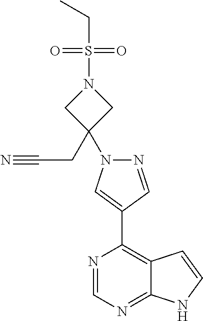 [SMILES: CCS(=O)(=O)N1CC(CC#N)(n2cc(-c3ncnc4[nH]ccc34)cn2)C1]

XIf. FFP104

Using the methods described herein, FFP104 can be administered to the subject. FFP104 is an anti-CD40 monoclonal antibody.

### XII. Antifibrotic Therapy

Using the methods described herein, an antifibrotic therapy can be administered to the subject. In some embodiments, the antifibrotic therapy is a vitamin D receptor (VDR) agonist or simtuzumab.

XIIa. VDR Agonist

Using the methods described herein, a VDR agonist can be administered to the subject. Exemplary VDR agonists include but are not limited to the compounds known by the INN names of seocalcitol, elocalcitol, and calcipotriol.

XIIai. Seocalcitol

Using the methods described herein, seocalcitol can be administered to the subject. Seocalcitol is the INN for the compound with the chemical structure depicted below.

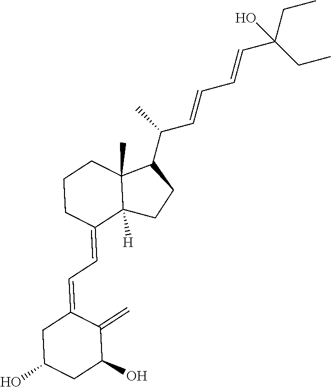

XIIaii. Elocalcitol

Using the methods described herein, elocalcitol can be administered to the subject. Elocalcitol is INN for the compound with the chemical structure depicted below.

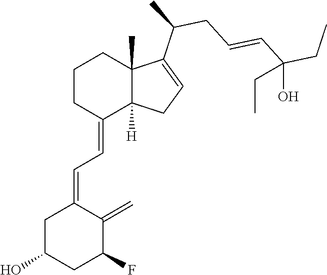 [SMILES: C=C1/C(=C\C=C2/CCC[C@]3(C)C([C@@H](C)C/C=C/C(O)(CC)CC)=CC[C@@H]23)C[C@@H](O)C[C@@H]1F]

XIIaiii. Calcipotriol

Using the methods described herein, calcipotriol can be administered to the subject. Calcipotriol is INN for the compound with the chemical structure depicted below.

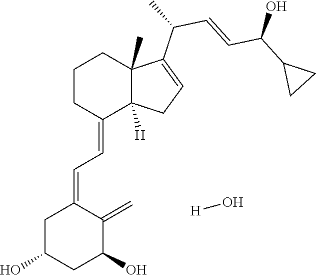 [SMILES: C=C1/C(=C\C=C2/CCC[C@]3(C)C([C@H](C)/C=C/[C@@H](O)C4CC4)=CC[C@@H]23)C[C@@H](O)C[C@@H]1O ; O]

XIIb. Simtuzumab

Using the methods described herein, simtuzumab can be administered to the subject. Simtuzumab is the INN for the antibody also known by the code name of GS-6624 and with the chemical formula of C₅₅₈H₁₀₁₃₄N₁₇₃₆O₂₀₃₇S₅₀.

### XIII. Nicotinamide Adenine Dinucleotide Phosphate Oxidase (NOX) Inhibitor

Using the methods described herein, a NOX inhibitor can be administered to the subject. In some embodiments, the NOX inhibitor is setanaxib.

XIIIa. Setanaxib

Using the methods described herein, setanaxib can be administered to the subject. Setanaxib is the INN for the compound known by the code name of GKT831. Setanaxib has the chemical structure depicted below.

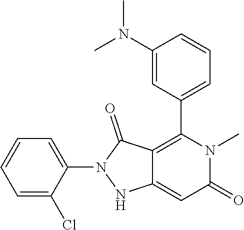 [SMILES: CN(C)c1cccc(-c2c3c(=O)n(-c4ccccc4Cl)[nH]c3cc(=O)n2C)c1]

### Kits

The compositions described herein can be provided in a kit for use in treating a glycogen storage disorder (e.g., Pompe disease). In some embodiments, the kit may include one or more viral vectors as described herein. The kit can include a package insert that instructs a user of the kit, such as a physician of skill in the art, to perform any one of the methods described herein. For example, in some embodiments, the kit may include a package insert that instructs a user of the kit to administer the viral vector to a patient. The kit may optionally include a syringe or other device for administering the composition. In some embodiments, the kit may include one or more additional therapeutic agents.

In some embodiments, the kit may include one or more anti-transaminitis agents as described herein. The kit can include a package insert that instructs a user of the kit, such as a physician of skill in the art, to perform any one of the methods described herein. For example, in some embodiments, the kit may include a package insert that instructs a user of the kit to administer the anti-transaminitis agent to a patient. The kit may optionally include a syringe or other device for administering the composition. In some embodiments, the kit may include one or more additional therapeutic agents.

### Recommended Clinical Parameters for Monitoring a Patient for Development of Transaminasemia or Hyperbilirubinemia

In some embodiments, a patient is monitored for the development of transaminasemia by a blood test (e.g., an LFT), as descried herein.

In some embodiments, a patient is monitored for the development of hyperbilirubinemia by a blood test (e.g., bilirubin test), as descried herein.

In some embodiments, a patient is monitored for the development of transaminasemia, and if the patient exhibits transaminasemia or one or more symptoms thereof, the patient is administered an anti-transaminitis agent.

In some embodiments, a patient is monitored for the development of hyperbilirubinemia, and if the patient exhibits hyperbilirubinemia or one or more symptoms thereof, the patient is administered an anti-transaminitis agent.

In some embodiments, a patient is monitored for the development of transaminasemia or hyperbilirubinemia by a blood test (e.g., a serum acid bile test or a liver function test). In some embodiments, a patient is monitored for the development of transaminasemia or hyperbilirubinemia by a blood test (e.g., a serum acid bile test or a liver function test), and if the patient exhibits transaminasemia or hyperbilirubinemia or one or more symptoms thereof, the patient is administered an anti-transaminitis agent.

In some embodiments, the patient is determined to exhibit transaminasemia, hyperbilirubinemia, or one or more symptoms thereof by a finding that the patient exhibits a parameter (e.g., a liver transaminase) in blood test (e.g., an LFT) that is increased relative to a reference level.

In some embodiments, the patient is determined to exhibit transaminasemia, hyperbilirubinemia, or one or more symptoms thereof by a finding that the patient exhibits a liver transaminase (e.g., ASP level or ALT level)) level in a blood test (e.g., LFT) that is increased relative to a reference level.

In some embodiments, the blood test is a liver function test.

In some embodiments, a patient is monitored for the development of transaminasemia or hyperbilirubinemia by a liver function test, and if the patient exhibits transaminasemia or hyperbilirubinemia or one or more symptoms thereof, the patient is administered an anti-transaminitis agent.

### Liver Function Test

In some embodiments, a patient is monitored for the development of transaminasemia or hyperbilirubinemia with an LFT. In some embodiments, a patient is monitored for the development of transaminasemia, hyperbilirubinemia, or one or more symptoms thereof, and if the patient exhibits transaminasemia or hyperbilirubinemia or one or more symptoms thereof, the patient is administered an anti-transaminitis agent. In some embodiments, a patient is monitored for the development of transaminasemia or hyperbilirubinemia with an LFT, and if the patient exhibits transaminasemia or hyperbilirubinemia or one or more symptoms thereof, the patient is administered an anti-transaminitis agent.

In some embodiments, it is determined that a patient exhibits transaminasemia, hyperbilirubinemia, or one or more symptoms thereof and is administered an anti-transaminitis agent when a parameter (e.g., ASP level or AST level) of the patient's LFT is greater than the age-adjusted norm, as descried herein.

### Aspartate Aminotransferase

In some embodiments, a patient is monitored for the development of transaminasemia or hyperbilirubinemia by measuring the patient's AST level in an LFT. In some embodiments, a patient is monitored for the development of transaminasemia, hyperbilirubinemia, or one or more symptoms thereof, and if the patient exhibits transaminasemia or hyperbilirubinemia or one or more symptoms thereof, the patient is administered an anti-transaminitis agent. In some embodiments, a patient is monitored for the development of transaminasemia or hyperbilirubinemia by measuring the patient's AST level in an LFT, and if the patient exhibits transaminasemia or hyperbilirubinemia or one or more symptoms thereof, the patient is administered an anti-transaminitis agent.

In some embodiments, a patient is monitored for the development of transaminasemia or hyperbilirubinemia by measuring the patient's AST level in an LFT and it is determined that a patient exhibits transaminasemia, hyperbilirubinemia, or one or more symptoms thereof and is administered an anti-transaminitis agent when the patient's AST level is greater than the norm.

In some embodiments, it is determined that a patient exhibits transaminasemia, hyperbilirubinemia, or one or more symptoms thereof when the patient's AST level is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

In some embodiments, it is determined that a patient exhibits transaminasemia, hyperbilirubinemia, or one or more symptoms thereof and is administered an anti-transaminitis agent when the patient's AST level is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Alanine Aminotransferase

In some embodiments, a patient is monitored for the development of transaminasemia or hyperbilirubinemia by measuring the patient's ALT level in an LFT. In some embodiments, a patient is monitored for the development of transaminasemia, hyperbilirubinemia, or one or more symptoms thereof, and if the patient exhibits transaminasemia or hyperbilirubinemia or one or more symptoms thereof, the patient is administered an anti-transaminitis agent. In some embodiments, a patient is monitored for the development of transaminasemia or hyperbilirubinemia by measuring the patient's ALT level in an LFT, and if the patient exhibits transaminasemia or hyperbilirubinemia or one or more symptoms thereof, the patient is administered an anti-transaminitis agent.

In some embodiments, a patient is monitored for the development of transaminasemia or hyperbilirubinemia by measuring the patient's ALT level in an LFT and it is determined that a patient exhibits transaminasemia, hyperbilirubinemia, or one or more symptoms thereof and is administered an anti-transaminitis agent when the patient's ALT level is greater than the norm.

In some embodiments, it is determined that a patient exhibits transaminasemia or one or more symptoms thereof when the patient's ALT level is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

In some embodiments, it is determined that a patient exhibits transaminasemia or one or more symptoms thereof and is administered an anti-transaminitis agent when the patient's ALT level is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Recommended Clinical Parameters for Monitoring a Patient for Development of Transaminasemia

### Blood Test

In some embodiments, a patient is monitored for the development of transaminasemia with a blood test (e.g., LFT or a bilirubin test). In some embodiments, a patient is monitored for the development of transaminasemia, and if the patient exhibits transaminasemia or one or more symptoms thereof, the patient is administered an anti-transaminitis agent. In some embodiments, a patient is monitored for the development of transaminasemia with an LFT, and if the patient exhibits transaminasemia or one or more symptoms thereof, the patient is administered an anti-transaminitis agent.

In some embodiments, it is determined that a patient exhibits transaminasemia or one or more symptoms thereof and is administered an anti-transaminitis agent when one or more parameters (e.g., GGT level, ASP level, AST level, ALT level, and bilirubin level) of the patient's blood test (e.g., a LFT or a bilirubin test) is greater than the age-adjusted norm, as descried herein.

### Liver Function Test

In some embodiments, a patient is monitored for the development of transaminasemia with an LFT. In some embodiments, a patient is monitored for the development of transaminasemia, and if the patient exhibits transaminasemia or one or more symptoms thereof, the patient is administered an anti-transaminitis agent. In some embodiments, a patient is monitored for the development of transaminasemia with an LFT, and if the patient exhibits transaminasemia or one or more symptoms thereof, the patient is administered an anti-transaminitis agent.

In some embodiments, it is determined that a patient exhibits transaminasemia or one or more symptoms thereof and is administered an anti-transaminitis agent when one or more parameters (e.g., GGT level, ASP level, AST level, and ALT level) of the patient's LFT is greater than the age-adjusted norm, as descried herein.

### Aspartate Aminotransferase

In some embodiments, a patient is monitored for the development of transaminasemia by measuring the patient's AST level in an LFT. In some embodiments, a patient is monitored for the development of transaminasemia, and if the patient exhibits transaminasemia or one or more symptoms thereof, the patient is administered an anti-transaminitis agent. In some embodiments, a patient is monitored for the development of transaminasemia by measuring the patient's AST level in an LFT, and if the patient exhibits transaminasemia or one or more symptoms thereof, the patient is administered an anti-transaminitis agent.

In some embodiments, a patient is monitored for the development of transaminasemia by measuring the patient's AST level in an LFT and it is determined that a patient exhibits transaminasemia or one or more symptoms thereof and is administered an anti-transaminitis agent when the patient's AST level is greater than the norm.

In some embodiments, it is determined that a patient exhibits transaminasemia or one or more symptoms thereof and is administered an anti-transaminitis agent when the patient's AST level is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Alanine Aminotransferase

In some embodiments, a patient is monitored for the development of transaminasemia by measuring the patient's ALT level in an LFT. In some embodiments, a patient is monitored for the development of transaminasemia, and if the patient exhibits transaminasemia or one or more symptoms thereof, the patient is administered an anti-transaminitis agent. In some embodiments, a patient is monitored for the development of transaminasemia by measuring the patient's ALT level in an LFT, and if the patient exhibits transaminasemia or one or more symptoms thereof, the patient is administered an anti-transaminitis agent.

In some embodiments, a patient is monitored for the development of transaminasemia by measuring the patient's ALT level in an LFT and it is determined that a patient exhibits transaminasemia or one or more symptoms thereof and is administered an anti-transaminitis agent when the patient's ALT level is greater than the norm.

In some embodiments, it is determined that a patient exhibits transaminasemia or one or more symptoms thereof and is administered an anti-transaminitis agent when the patient's ALT level is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Recommended Clinical Parameters for Monitoring a Patient for Development of Hyperbilirubinemia

### Bilirubin Test

In some embodiments, a patient is monitored for the development of hyperbilirubinemia. In some embodiments, a patient is monitored for the development of hyperbilirubinemia with a bilirubin test. In some embodiments, a patient is monitored for the development of hyperbilirubinemia, and if the patient exhibits hyperbilirubinemia or one or more symptoms thereof, the patient is administered an anti-transaminitis agent. In some embodiments, a patient is monitored for the development of hyperbilirubinemia with a bilirubin test, and if the patient exhibits hyperbilirubinemia or one or more symptoms thereof, the patient is administered an anti-transaminitis agent.

In some embodiments, it is determined that a patient exhibits hyperbilirubinemia or one or more symptoms thereof and is administered an anti-transaminitis agent when the patient exhibits a bilirubin level that is greater than the norm.

In some embodiments, it is determined that a patient exhibits hyperbilirubinemia or one or more symptoms thereof and is administered an anti-transaminitis agent when the patient's total bilirubin level is greater than 1.2 mg/dL (e.g., 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, and 100 mg/dL).

In some embodiments, it is determined that a patient exhibits hyperbilirubinemia or one or more symptoms thereof and is administered an anti-transaminitis agent when the patient's direct bilirubin level is greater than 0.2 mg/dL (e.g., 0.2 mg/dL, 0.3 mg/dL, 0.4 mg/dL, 0.5 mg/dL, 0.6 mg/dL, 0.7 mg/dL, 0.8 mg/dL, 0.9 mg/dL, 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, and 100 mg/dL).

In some embodiments, the patient is determined to exhibit hyperbilirubinemia or one or more symptoms thereof and is administered an anti-transaminitis agent when the patient exhibits a bilirubin level that is greater than 1 mg/dL (e.g., greater than 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, or 100 mg/dL) in a bilirubin test.

Recommended Clinical Parameters for Determining that a Patient Exhibits Transaminasemia or Hyperbilirubinemia or a Symptom Thereof

In some embodiments, it is determined that the patient exhibits transaminasemia, hyperbilirubinemia, or one or more symptoms thereof by determining that one or more parameters (e.g., GGT level, ASP level, AST level, and ALT level) of the patient's blood test (e.g., an LFT) is greater than or less than the age-adjusted norm, as descried herein, and the patient is administered an anti-transaminitis agent.

In some embodiments, it is determined that the patient exhibits transaminasemia, hyperbilirubinemia, or one or more symptoms thereof by determining that one or more parameters (e.g., bilirubin level) of the patient's blood test (e.g., bilirubin test) is greater than the norm, as descried herein, and the patient is administered an anti-transaminitis agent.

In some embodiments, it is determined that a patient exhibits transaminasemia, hyperbilirubinemia, or one or more symptoms thereof and is administered an anti-transaminitis agent when a parameter (e.g., ASP level or AST level) of the patient's LFT is greater than the age-adjusted norm, as descried herein.

### Liver Function Test

In some embodiments, it is determined that a patient exhibits transaminasemia, hyperbilirubinemia, or one or more symptoms thereof and is administered an anti-transaminitis agent when a parameter (e.g., ASP level or AST level) of the patient's LFT is greater than the age-adjusted norm, as descried herein.

### Aspartate Aminotransferase

In some embodiments, a patient is monitored for the development of transaminasemia or hyperbilirubinemia by measuring the patient's AST level in an LFT and it is determined that a patient exhibits transaminasemia, hyperbilirubinemia, or one or more symptoms thereof and is administered an anti-transaminitis agent when the patient's AST level is greater than the norm.

In some embodiments, it is determined that a patient exhibits transaminasemia, hyperbilirubinemia, or one or more symptoms thereof when the patient's AST level is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

In some embodiments, it is determined that a patient exhibits transaminasemia, hyperbilirubinemia, or one or more symptoms thereof and is administered an anti-transaminitis agent when the patient's AST level is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Alanine Aminotransferase

In some embodiments, a patient is monitored for the development of transaminasemia or hyperbilirubinemia by measuring the patient's ALT level in an LFT and it is determined that a patient exhibits transaminasemia, hyperbilirubinemia, or one or more symptoms thereof and is administered an anti-transaminitis agent when the patient's ALT level is greater than the norm.

In some embodiments, it is determined that a patient exhibits transaminasemia or one or more symptoms thereof when the patient's ALT level is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

In some embodiments, it is determined that a patient exhibits transaminasemia or one or more symptoms thereof and is administered an anti-transaminitis agent when the patient's ALT level is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

Recommended Clinical Parameters for Determining that a Patient Exhibits Transaminasemia or a Symptom Thereof

### Blood Test

In some embodiments, a patient is determined to exhibit transaminasemia or one or more symptoms thereof and is administered an anti-transaminitis agent when one or more parameters (e.g., GGT level, ASP level, AST level, ALT level, and bilirubin level) of the patient's blood test (e.g., a LFT or a bilirubin test) is greater than the age-adjusted norm, as descried herein.

a. Liver Function Test

In some embodiments, a patient is determined to exhibit transaminasemia or one or more symptoms thereof and is administered an anti-transaminitis agent when one or more parameters (e.g., GGT level, ASP level, AST level, and ALT level) of the patient's LFT is greater than the age-adjusted norm, as descried herein.

### Aspartate Aminotransferase

In some embodiments, a patient is determined to exhibit transaminasemia or one or more symptoms thereof and is administered an anti-transaminitis agent when the patient exhibits an AST level, as measured in a LFT, that is greater than the norm.

In some embodiments, a is determined to exhibit transaminasemia or one or more symptoms thereof and is administered an anti-transaminitis agent when the patient's AST level is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

### Alanine Aminotransferase

In some embodiments, a patient is determined to exhibit transaminasemia or one or more symptoms thereof and is administered an anti-transaminitis agent when the patient exhibits an ALT level, as measured in a LFT, that is greater than the norm.

In some embodiments, a patient is determined to exhibit transaminasemia or one or more symptoms thereof and is administered an anti-transaminitis agent when the patient's ALT level is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L).

Recommended Clinical Parameters for Determining that a Patient Exhibits Hyperbilirubinemia or a Symptom Thereof

### Bilirubin Test

In some embodiments, a patient is determined to exhibit hyperbilirubinemia or one or more symptoms thereof and is administered an anti-transaminitis agent when the patient exhibits a bilirubin level, as measured in a blood test (e.g., a bilirubin test), that is greater than the norm.

In some embodiments, a patient is determined to exhibit hyperbilirubinemia or one or more symptoms thereof and is administered an anti-transaminitis agent when the patient's total bilirubin level is greater than 1.2 mg/dL (e.g., 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, and 100 mg/dL).

In some embodiments, a patient is determined to exhibit hyperbilirubinemia or one or more symptoms thereof and is administered an anti-transaminitis agent when the patient's direct bilirubin level is greater than 0.2 mg/dL (e.g., 0.2 mg/dL, 0.3 mg/dL, 0.4 mg/dL, 0.5 mg/dL, 0.6 mg/dL, 0.7 mg/dL, 0.8 mg/dL, 0.9 mg/dL, 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, and 100 mg/dL).

In some embodiments, the patient is determined to exhibit hyperbilirubinemia or one or more symptoms thereof and is administered an anti-transaminitis agent when the patient exhibits a bilirubin level that is greater than 1 mg/dL (e.g., greater than 1 mg/dL, 1.1 mg/dL, 1.2 mg/dL, 1.3 mg/dL, 1.4 mg/dL, 1.5 mg/dL, 1.6 mg/dL, 1.7 mg/dL, 1.8 mg/dL, 1.9 mg/dL, 2 mg/dL, 2.1 mg/dL, 2.2 mg/dL, 2.3 mg/dL, 2.4 mg/dL, 2.5 mg/dL, 2.6 mg/dL, 2.7 mg/dL, 2.8 mg/dL, 2.9 mg/dL, 3 mg/dL, 3.1 mg/dL, 3.2 mg/dL, 3.3. mg/dL, 3.4 mg/dL, 3.5 mg/dL, 3.6 mg/dL, 3.7 mg/dL, 3.8 mg/dL, 3.9 mg/dL, 4 mg/dL, 4.1 mg/dL, 4.2 mg/dL, 4.3 mg/dL, 4.4 mg/dL, 4.5 mg/dL, 4.6 mg/dL, 4.7 mg/dL, 4.8 mg/dL, 4.9 mg/dL, 5 mg/dL, 10 mg/dL, 15 mg/dL, 20 mg/dL, 30 mg/dL, 40 mg/dL, 50 mg/dL, 60 mg/dL, 70 mg/dL, 80 mg/dL, 90 mg/dL, or 100 mg/dL) in a bilirubin test.

### EXAMPLES

The following examples are put forth to provide those of ordinary skill in the art with a description of how the compositions and methods described herein may be used and evaluated and are intended to be purely exemplary of the invention and are not intended to limit the scope of what the inventors regard as their invention.

### Example 1. Emergent Liver Disorders Post Administration of a Viral Vector Encoding Acid Alpha-Glucosidase and Prednisolone as Prophylaxis and Treatment for Transaminitis

The objective of this study was to examine the potential adverse effects of an AAV8 vector containing a GAA transgene operably linked to a muscle creatine kinase (MCK) promoter in human patients having a glycogen storage disorder (e.g., Pompe disease). Pompe disease is a monogenic, autosomal recessive disease caused by mutations in the gene encoding the lysosomal enzyme GAA, which normally degrades lysosomal glycogen. Deficiency in functional GAA leads to accumulation of glycogen in the lysosomes of all cells in the body, with pathophysiological damage to skeletal and cardiac muscles. Pompe disease spans a continuum of disease severity in which age of disease onset, degree of myopathy and extent of organ involvement correlate with residual enzyme activity. Late-onset Pompe disease (LOPD) typically manifests as slowly progressive limb girdle myopathy with proximal muscle weakness and respiratory impairment. With disease progression LOPD can eventually result in impaired mobility and respiratory insufficiency requiring wheelchair use and a full-time ventilator.

### Materials and Methods

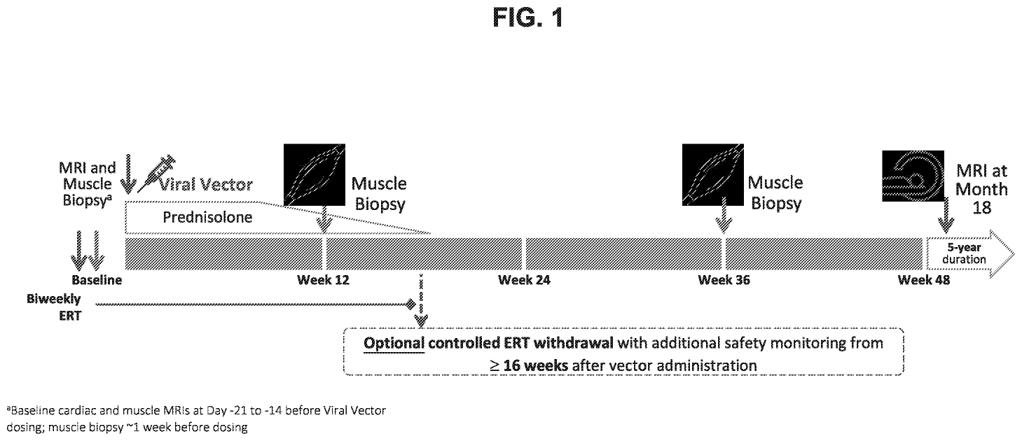

Four patients having Pompe disease and greater than or equal to 18 years old were administered a recombinant AAV vector serotype 8 expressing the recombinant human acid alpha glucosidase (rhGAA) gene specifically in the muscle, which is currently under clinical development for the treatment of LOPD. FORTIS (NCT04174105) is an ongoing multicenter, open-label, ascending dose Phase I/II first-in-human clinical trial to determine if the vector is safe and tolerable in adult subjects with LOPD. Subjects enrolled in FORTIS receive a one-time peripheral intravenous infusion of the vector described herein, followed by one year of frequent monitoring of clinical and biochemical endpoints including GAA activity and protein level in muscle and four years of long-term safety monitoring (FIG. 1). Additional inclusion criteria included being either ambulant or nonambulant and having received enzyme replacement therapy (ERT) with rhGAA for greater than or equal to two years. Furthermore, inclusion criteria included being on a standard dose (e.g., at least 20 mg/kg every two weeks) of ERT with rhGAA for at least the previous 6 months, the ability to be upright with a forced vital capacity (FVC) of greater than or equal to 30% of predicted normal value. Exclusion criteria included patients who are currently participating in an interventional study or have received gene or cell therapy, positive for high titers of AAV8 neutralizing or GAA antibodies, receiving immune-modulating agents within 90 days before dosing (with the use of inhaled corticosteroids allowed as an exception), at high risk for severe allergic reaction to rhGAA (e.g., previous moderate to severe anaphylactic reaction to ERT and/or a history of sustained high immunoglobulin antibody titers to ERT), have an active viral infection based on clinical observation, have a history of clinically important cardiac conditions (e.g., an ejection fraction (EF) of less than 40%) or have symptoms or signs of cardiomyopathy, clinically significant underlying liver disease, or contraindication to study drug or ingredients or corticosteroids.

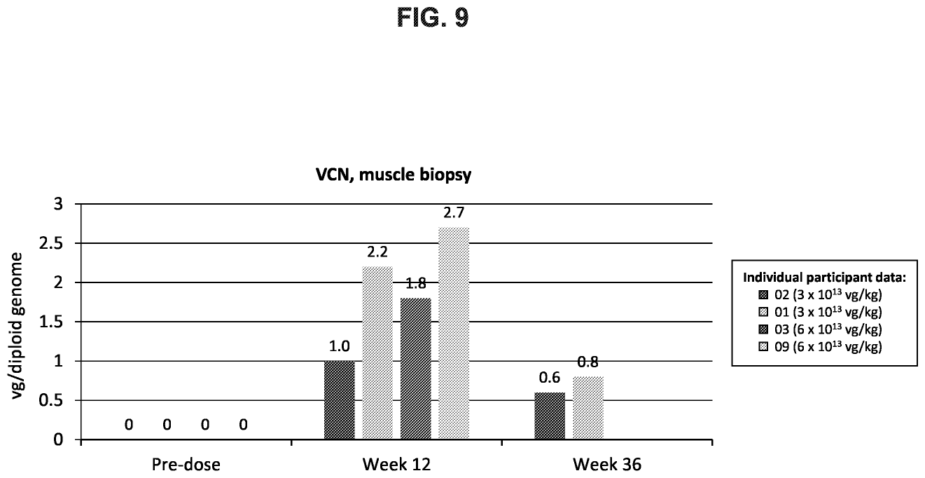

Primary endpoints in the study include monitoring the frequency of adverse events, serious adverse events and changes from baseline in relevant clinical tests (e.g., clinical chemistry, hematology, coagulation parameters, and urinalysis), elevations in cardiac enzymes, and potential immune responses. Secondary assessments include change from baseline in GAA protein expression and enzyme activity in muscle (Week 12; see e.g., FIG. 9) as well as an evaluation of improvements in respiratory, endurance/functional strength, and quality of life measures.

A summary of the participants and doses administered are presented in Table 1.

TABLE 1
Study Outline by Dose Level of AAV encoding GAA

| | | | Age at | AAV | Baseline | Total dose | | Follow-up |
|---|---|---|---|---|---|---|---|---|
| | Participant | Age | genetic | dose | weight | administered | Date of AAV | time |
| Cohort | ID | (y)/Sex | diagnosis (y) | (vg/kg) | (kg) | (vg) | administration | (weeks) |
| 1 | 202-2002 | 48/F | 30 | 3.0 × 10¹³ | 49.8 | 1.497 × 10¹⁵ | 18 Mar. 2021 | 53 |
| 1 | 201-2001 | 52/M | 48 | 3.0 × 10¹³ | 81.8 | 2.430 × 10¹⁵ | 26 May 2021 | 43 |
| 2 | 203-2003 | 66/M | 53 | 6.0 × 10¹³ | 70.3 | 4.218 × 10¹⁵ | 1 Sep. 2021 | 29 |
| 2 | 204-2009 | 49/F | 38 | 6.0 × 10¹³ | 77.1 | 4.626 × 10¹⁵ | 17 Nov. 2021 | 18 |

### Results

These interim data focus on safety findings for Cohort 1 and Cohort 2.

Four adult subjects with LOPD have received an AAV described herein, with two subjects dosed with the clinical starting dose of 3×10¹³ vg/kg and two subjects dosed with the escalated dose of 6×10¹³ vg/kg. The infusions were generally well-tolerated, and no serious adverse events (SAEs) related to the study drug were reported as of the data cut-off for this analysis. Since the data cut-off, one Treatment-Emergent Adverse Event (TEAE), tingling hands and feet, has been reclassified as peripheral sensory neuropathy and designated as a SAE. Grade 1 TEAEs were defined as mild; asymptomatic or mild symptoms; clinical or diagnostic observations only; no intervention indicated. Grade 2 TEAEs were defined as moderate; minimal, local or noninvasive intervention indicated. Grade 3 TEAEs were defined as severe or medically significant but not immediately life threatening; hospitalization or prolongation of hospitalization indicated; disabling. Grade 4 TEAEs were defined as life-threatening consequences; urgent intervention indicated, while Grade 5 TEAEs were defined as death related to AE. Subjects dosed in the study experienced TEAEs, defined as adverse effects (AEs) that occurred after administration of the AAV encoding GAA described herein. Table 2 presents the most frequent TEAEs that were reported in the subjects. TEAEs occurring at a severity of Grade 3 in 1 subject included transaminasemia-related adverse events, including an increase in alanine aminotransferase (ALT). TEAEs occurring at a severity of Grade 2 in 3 subjects included transaminasemia-related adverse events, including an increase in alanine aminotransferase (ALT) and aspartate aminotransferase (AST), as well as nausea. All other observed TEAEs were Grade 1 TEAEs and considered to be unrelated to study treatment. There were no AAV infusion-related AEs, and no deaths have been reported.

TABLE 2
TEAEs Reported in Subjects, by Preferred Dose Cohort

| | Cohort 1 | | Cohort 2 | |
|---|---|---|---|---|
| | (3 × 10¹³ vg/kg) | | (6 × 10¹³ vg/kg) | |
| | Patient | Patient | Patient | Patient |
| | 2002 | 2001 | 2003 | 2009 |
| Follow-up time, weeks | 53 | 43 | 29 | 18 |
| AII TEAE | 1 | 5 | 19 | 18 |
| Procedural pain | 1 | — | — | — |
| Headache | — | 1 | 1 | — |
| Ageusia | — | | 1 | 1 |
| ALT increased | — | 1 | 1 | 1 |
| AST increased | — | 1 | 1 | 1 |
| Decreased appetite | — | — | 1 | 1 |
| Constipation | — | — | 1 | 1 |
| Abdominal distension | — | — | 1 | — |
| Nausea | — | — | — | 1 |
| Diarrhea | — | — | — | 1 |
| Irritability | — | — | 1 | — |
| Palpitations | — | — | 2 | — |
| Night sweats | — | — | 1 | — |
| Cold sweat | — | — | 1 | — |
| Dyspnea | — | — | 1 | — |
| COVID-19 | — | 1 | 1 | — |
| Upper respiratory tract | — | — | 1 | — |
| congestion | | | | |
| Malaise | — | — | 1 | — |
| Fatigue | — | — | 1 | 1 |
| Insomnia | — | 1 | — | — |
| Anxiety | — | — | — | 1 |
| Hot flashes | — | — | 1 | — |
| Rash | — | — | 1 | 1 |
| Rash (arms and trunk) | — | — | — | 1 |
| Fever | — | — | — | 1 |
| Proteinuria | — | — | — | 1 |
| Right arm pain | — | — | — | 1 |
| Priuritis | — | — | — | 1 |
| Tingling hands and feet | — | — | — | 1 |
| Epistaxis | — | — | — | 1 |
| High blood pressure | — | — | — | 1 |

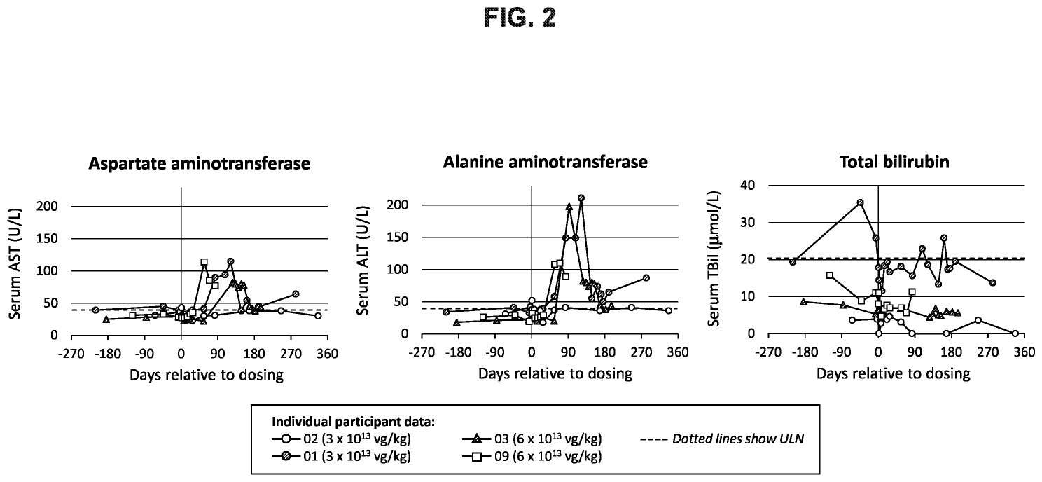

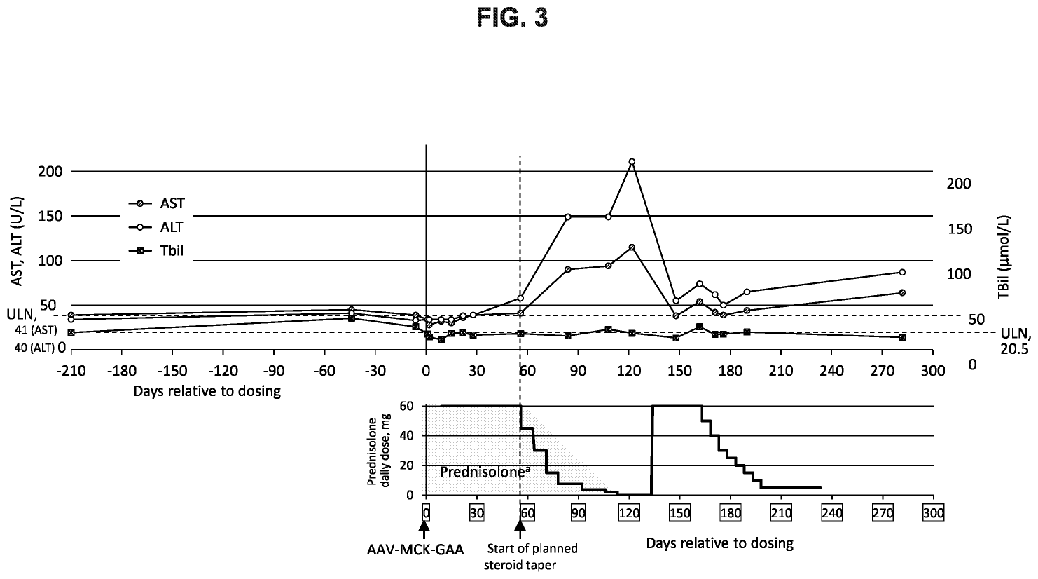

FIG. 2 is a set of graphs depicting the longitudinal observation of ALT and AST as well as bilirubin levels in patients dosed with an AAV8 vector containing a GAA transgene operably linked to a MCK promoter. One participant (2001) presented with baseline hyperbilirubinemia, which resolved post dosing but has subsequently been elevated intermittently (not considered an AE). The same patient showed a rise in transaminases after tapering of prednisolone prophylaxis, which responded well to re-initiation of corticosteroid treatment (FIG. 3).

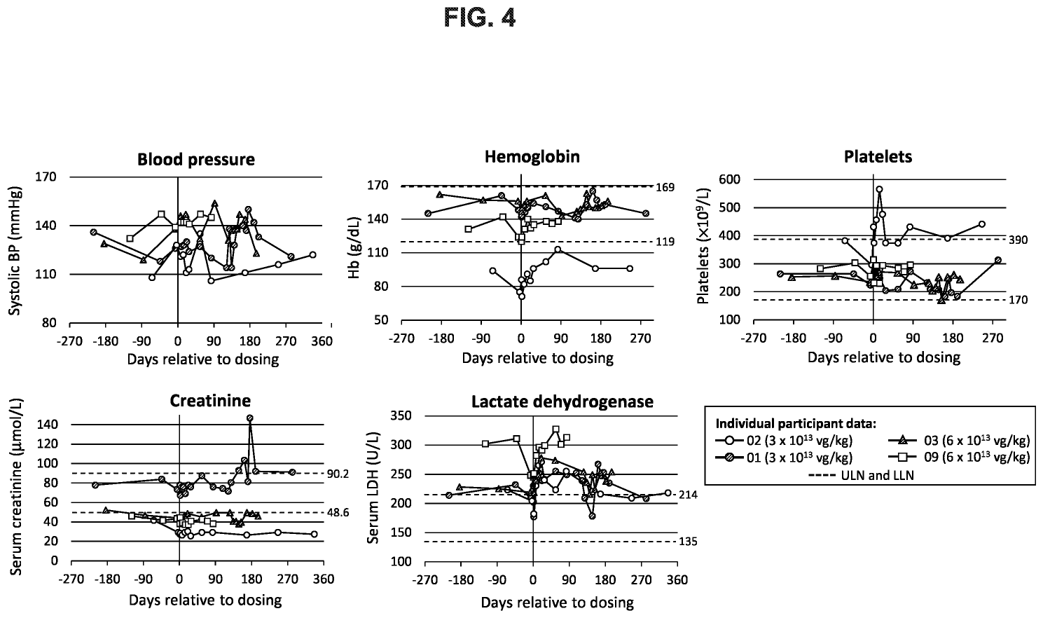

Safety monitoring for thrombotic microangiopathy (TMA) was conducted, and we found that there was no evidence of TMA in any of the participants (FIG. 4). Participant 2002 has a diagnosis of iron deficiency anemia, which likely accounts for her low Hb levels and thrombocytosis.

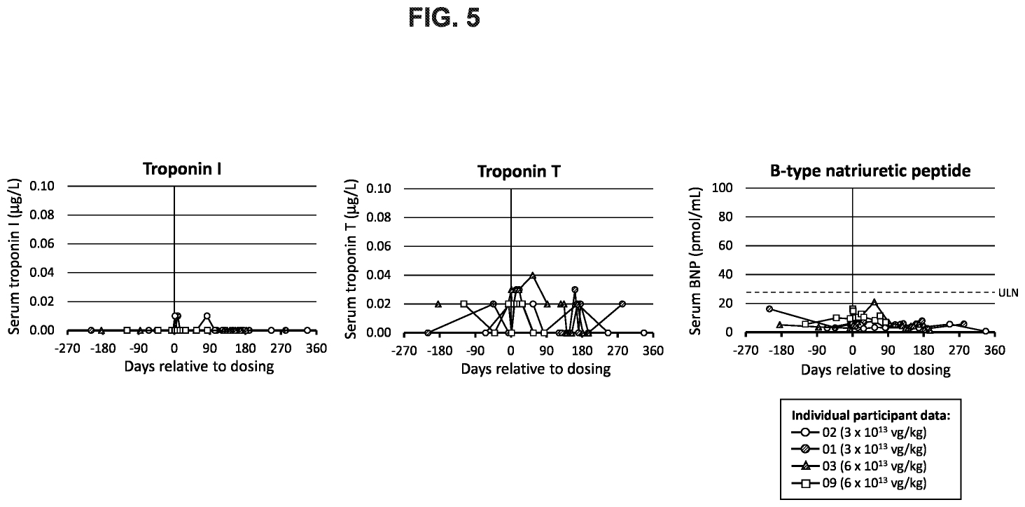

To evaluate cardiac safety, levels troponin I, troponin T, and B-type natriuretic peptide were monitored (FIG. 5). Serum troponin and B-type natriuretic peptide levels were minimal or below limits of detection before dosing and at all timepoints through up to one year of follow-up to date (n=4). Additionally, electrocardiogram (ECG) and echocardiogram results indicated that in Cohort 1, no abnormalities in cardiac parameters were observed (heart rate, PR, QRS, QT, QTc, and echo) at screening or any time following dosing. In Cohort 2, Participant 2003 presented at screening (Day −184) and run-in (Day −86) assessments with abnormal but not clinically significant ECG and possible left atrial enlargement but was assessed as normal at baseline (Day −7). Subsequently, non-clinically significant findings were reported at some, but not all post-dose assessments. Overall, no cardiac safety events were observed in the first four participants.

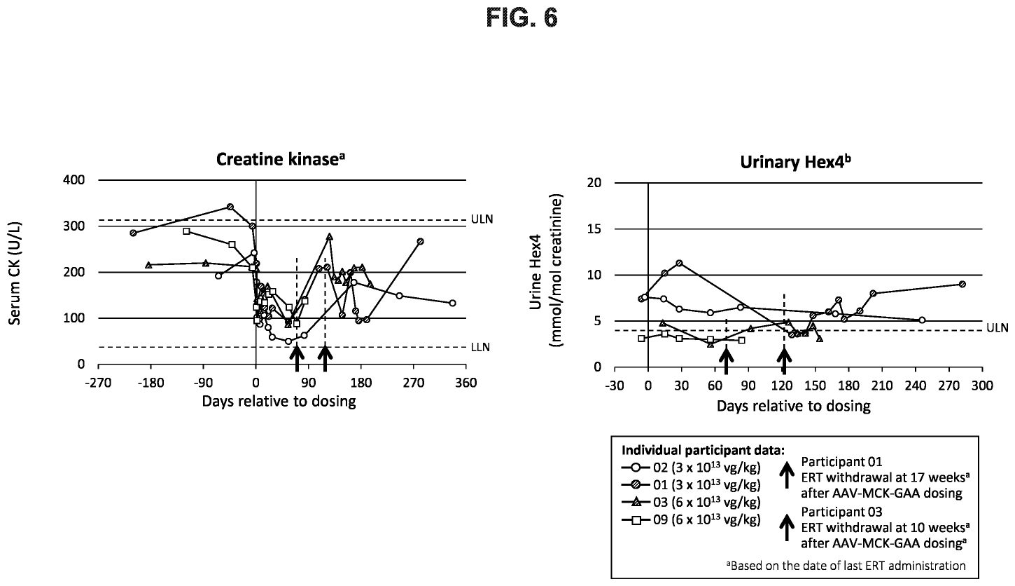

To evaluate biomarkers of disease, we measured blood creatine kinase and urine Hex4. Creatine kinase (CK) is a sensitive marker for muscle disease, although it is nonspecific for Pompe disease. Blood CK levels are elevated in most individuals with LOPD (e.g., 1.5 to 15×ULN) (e.g., normal range 38-174 U/L in men; 90-140 U/L in women). Urine Hex4 is often elevated in glycogen storage diseases and muscle disorders; although non-specific as a diagnostic test for Pompe disease, it can be useful for monitoring response to treatment (e.g., Hex4 concentrations are measured relative to creatine and reported as a normalized Hex4 in mmol/mol creatinine; normally <4). All four participants showed stabilization of biomarkers of disease, even after withdrawal of enzyme replacement therapy in participants 2001 and 2003 (FIG. 6). For participants in Cohort 1 and Cohort 2, we observed that creatine kinase levels show an early decline to levels within normal limits in all four participants (FIG. 6). It was also observed that urinary Hex4 levels are generally stable over the first months after dosing (FIG. 6).

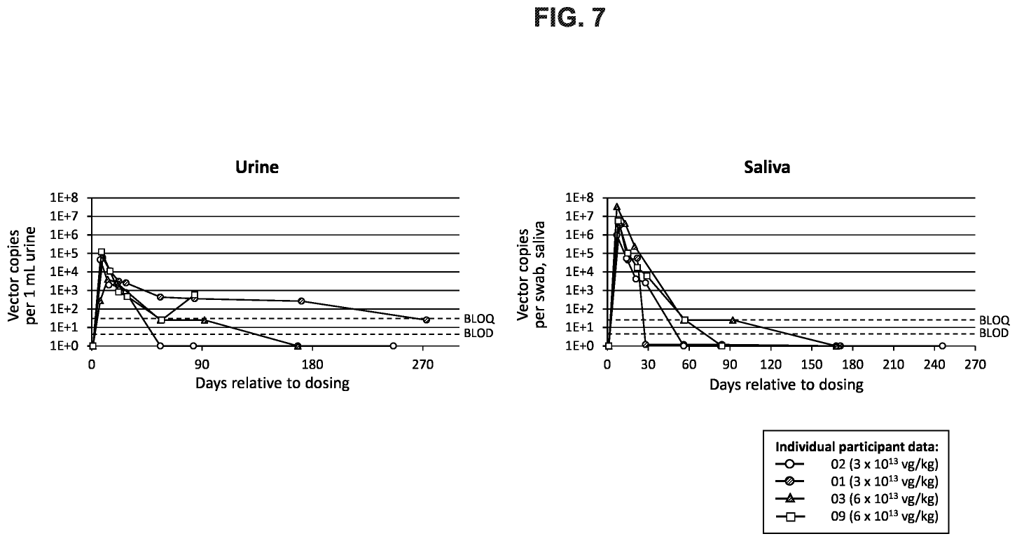

To evaluate vector shedding, urine and saliva samples were taken. In the participants dosed, vector shedding in urine and saliva peaked in the first 2 weeks after infusion of an AAV described herein (FIG. 7). Over the subsequent 2 months, vector shedding declined to levels below the limit of quantification (BLOQ) or below the limit of detection (BLOD).

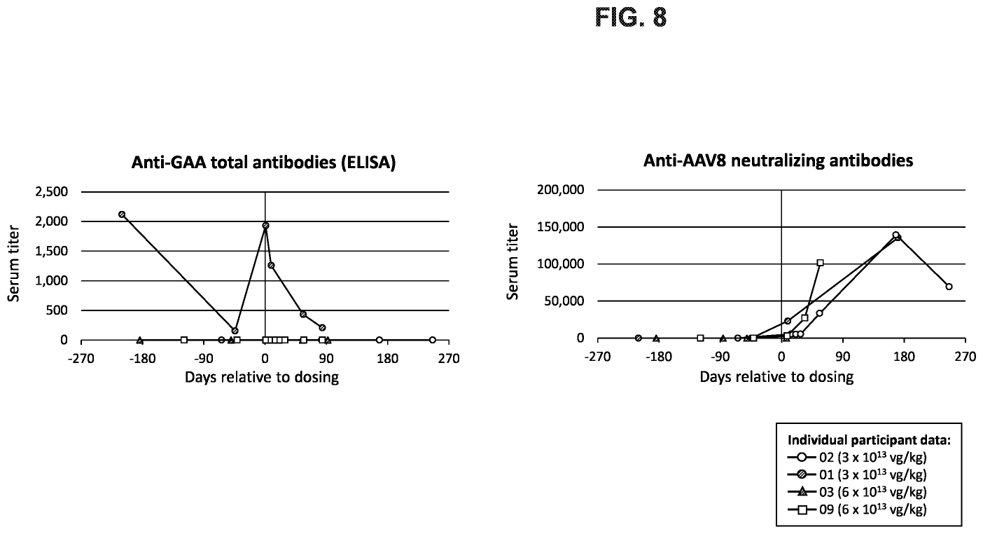

To evaluate the humoral immune response to a pseudotyped AAV2/8 vector including a nucleic acid sequence encoding an GAA gene operably linked to a MCK promoter, described herein, an enzyme-linked immunosorbent assay (ELISA) was performed using an antibody titer test against anti-GAA total or anti-AAV8 neutralizing antibodies, respectively (FIG. 8). Regarding the anti-GAA total antibodies, it was observed that three participants (2002, 2003, and 2009) had no detectable antibodies prior to infusion of the AAV-GAA vector and continue to have no antibodies up to day 246 post infusion. One participant (2001) has had varying levels of anti-GAA titers since screening with no increase following infusion of the AAV. With the anti-AAV neutralizing antibodies, it was observed that neutralizing antibodies to AAV8 increase as expected following infusion of the AAV.

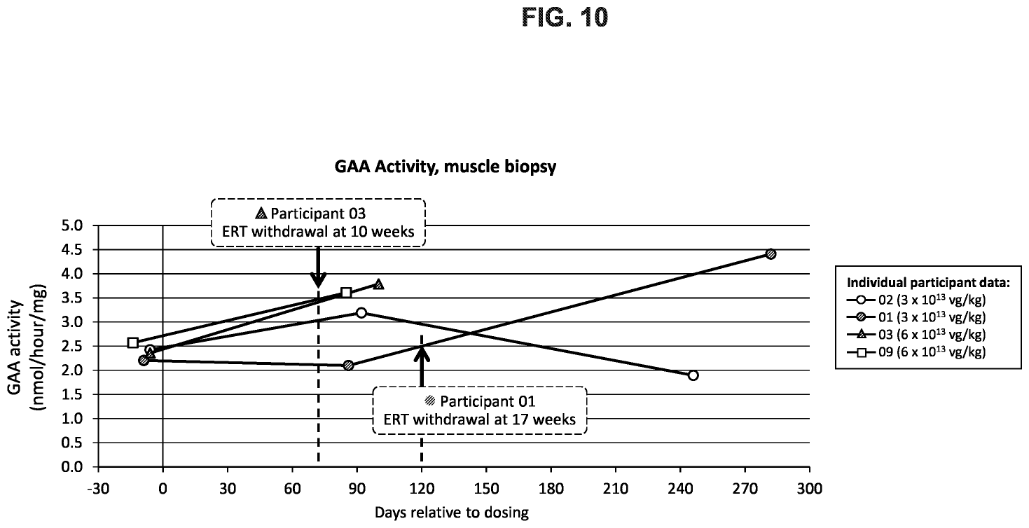

To confirm transduction of the AAV in target tissues, a muscle biopsy was taken, and a vector copy number assay was performed. Both participants in Cohort 1 and both participants in Cohort 2 showed transduction of the gene of interest in the muscle by 12 weeks post infusion of the AAV (FIG. 9). Week 36 muscle biopsies in Cohort 1 showed a sustained presence of the vector genomes (FIG. 9). Protein expression was evaluated in the target tissue for all four participants. Muscle biopsies showed increases in GAA activity in all four participants (FIG. 10). Withdrawal from enzyme replacement therapy was achieved for two participants (2001 and 2003) following AAV administration (FIG. 10).

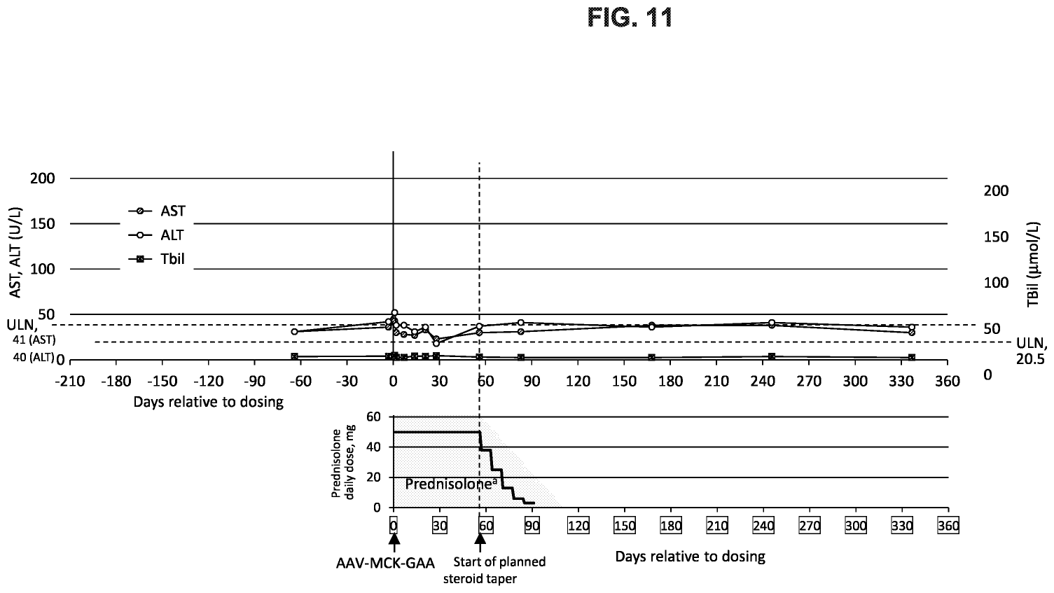

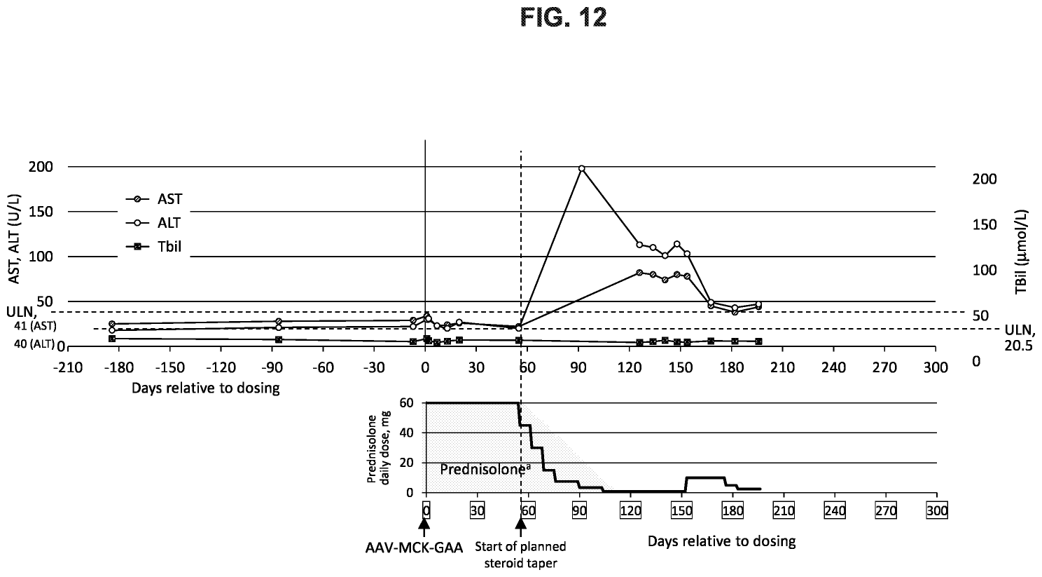

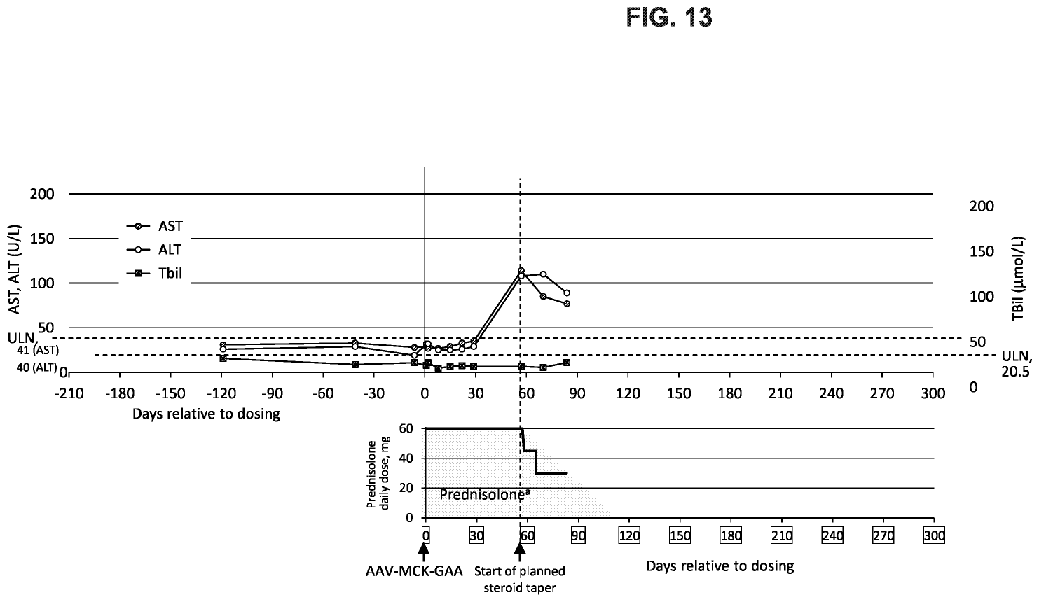

Liver transaminases and bilirubin were also monitored over time for patients 2002, 2003, and 2009 (FIG. 11-13). Patient 2002 was a female of low body weight who received a reduced dose of prophylactic prednisone. Liver function parameters remained within the normal range throughout steroid tapering and throughout the >1 year follow up period (FIG. 11). Similar to patient 2001, patients 2003 and 2009 showed a rise in transaminases upon tapering of prednisolone prophylaxis (FIGS. 12 and 13). For participant 2003, transaminitis was responsive to a slowing in the steroid tapering schedule and a small increment in steroid dose (FIG. 12). For participant 2009, transaminitis was responsive to a slowing in the steroid tapering schedule (FIG. 13). In these cases of transaminitis, there was no associated evidence of cholestasis or impaired synthetic function.

### Conclusion

In summary, we found that a combination therapy, including administration of a viral vector including a transgene encoding GAA and an anti-transaminitis agent (e.g., a corticosteroid, such as prednisolone), can serve as a prophylactic treatment for liver syndromes associated with the existing gene therapy approaches involving the delivery of GAA to patients in need thereof (e.g., patients with Pompe disease).

### Example 2. Treatment of Pompe Disease in Human Patients by Administration of the Viral Vector and an Anti-Transaminitis Agent

Using the compositions and methods of the disclosure, a patient (e.g., one year old or older) having LOPD may be administered the viral vector, for example, in a dose of from about 1×10¹³ vg/kg to about 3×10¹⁴ vg/kg (e.g., from about 1×10¹³ vg/kg to about 6×10¹³ vg/kg, from about 1×10¹³ vg/kg to about 5×10¹³ vg/kg, from about 1×10¹³ vg/kg to about 4×10¹³ vg/kg, from about 1×10¹³ vg/kg to about 3×10¹³ vg/kg, from about 2×10¹³ vg/kg to about 6×10¹³ vg/kg, from about 2×10¹³ vg/kg to about 5×10¹³ vg/kg, or from about 2×10¹³ vg/kg to about 4×10¹³ vg/kg) and an anti-transaminitis agent (e.g., prednisolone). The anti-transaminitis agent may be, for example, administered as doses of about 0.1 mg/kg/dose to about 2 mg/kg/dose and by way of a unit dosage form including 5 mg, 10 mg, 15, mg, or 30 mg and/or from 1 mg/day to about 120 mg/day.

Upon administering the viral vector to the patient, the patient exhibits endogenous GAA activity of about 50% to about 200% of the endogenous GAA activity of a human of the same gender and similar body mass index that does not have Pompe disease.

### Example 3. Treatment of Pompe Disease in Human Patients that are One Year Old or Older by Administration of a Pseudotyped AAV2/8 Vector Including a Nucleic Acid Sequence Encoding an Acid Alpha-Glucosidase Gene Operably Linked to a MCK Promoter

Using the compositions and methods of the disclosure, a patient having LOPD that is greater than about one year old may be administered a pseudotyped AAV2/8 vector including a nucleic acid sequence encoding an GAA gene operably linked to a MCK promoter (e.g., the viral vector), for example, in a dose of from about 1×10¹³ vg/kg to about 3×10¹⁴ vg/kg (e.g., from about 1×10¹³ vg/kg to about 6×10¹³ vg/kg, from about 1×10¹³ vg/kg to about 5×10¹³ vg/kg, from about 1×10¹³ vg/kg to about 4×10¹³ vg/kg, from about 1×10¹³ vg/kg to about 3×10¹³ vg/kg, from about 2×10¹³ vg/kg to about 6×10¹³ vg/kg, from about 2×10¹³ vg/kg to about 5×10¹³ vg/kg, or from about 2×10¹³ vg/kg to about 4×10¹³ vg/kg). Afterwards, the patient may be monitored for the development of transaminitis, and if the patient is determined to exhibit transaminitis or one or more symptoms thereof, the patient is administered an anti-transaminitis agent (e.g., prednisolone). The anti-transaminitis agent may be, for example, administered in one or doses of about 0.1 mg/kg/dose to about 2 mg/kg/dose and by way of a unit dosage form including 5 mg, 10 mg, 15, mg, or 30 mg and/or from 1 mg/day to about 120 mg/day.

Upon administering the pseudotyped AAV2/8 vector including a nucleic acid sequence encoding an GAA gene operably linked to a MCK promoter (e.g., the viral vector) to the patient, the patient displays a change from baseline in quantitative analysis of GAA expression in a muscle biopsy. For example, the patient displays the change from baseline in quantitative analysis of GAA expression in a muscle biopsy by about 24 weeks (e.g., by about 20 weeks, 16 weeks, 12 weeks, 8 weeks, or 4 weeks) after administration of the pseudotyped AAV2/8 vector including a nucleic acid sequence encoding an GAA gene operably linked to a MCK promoter (e.g., the viral vector) to the patient. Upon administering the pseudotyped AAV2/8 vector including a nucleic acid sequence encoding an GAA gene operably linked to a MCK promoter (e.g., the viral vector) to the patient, the patient displays a reduction of glycogen accumulation in muscle tissue and/or in neuronal tissue by about 24 weeks (e.g., by about 20 weeks, 16 weeks, 12 weeks, 8 weeks, or 4 weeks) after administration of the pseudotyped AAV2/8 vector including a nucleic acid sequence encoding an GAA gene operably linked to a MCK promoter (e.g., the viral vector) to the patient.

### Example 4. Treatment of Pompe Disease in Human Patients that are Ten Years Old or Older by Administration of the Viral Vector

Using the compositions and methods of the disclosure, a patient having LOPD that is greater than about ten years old may be administered the viral vector, for example, in a dose of from about 1×10¹³ vg/kg to about 3×10⁴ vg/kg (e.g., from about 1×10¹³ vg/kg to about 6×10¹³ vg/kg, from about 1×10¹³ vg/kg to about 5×10¹³ vg/kg, from about 1×10¹³ vg/kg to about 4×10¹³ vg/kg, from about 1×10¹³ vg/kg to about 3×10¹³ vg/kg, from about 2×10¹³ vg/kg to about 6×10¹³ vg/kg, from about 2×10¹³ vg/kg to about 5×10¹³ vg/kg, or from about 2×10¹³ vg/kg to about 4×10¹³ vg/kg). Afterwards, the patient may be monitored for the development of transaminitis, and if the patient is determined to exhibit transaminitis or one or more symptoms thereof, the patient is administered an anti-transaminitis agent (e.g., prednisolone), for example, in one or doses of about 0.1 mg/kg/dose to about 2 mg/kg/dose and by way of a unit dosage form including 5 mg, 10 mg, 15, mg, or 30 mg and/or from 1 mg/day to about 120 mg/day.

Upon administering the viral vector to the patient, the patient displays a change from baseline in quantitative analysis of GAA expression in a muscle biopsy. For example, the patient displays the change from baseline in quantitative analysis of GAA expression in a muscle biopsy by about 24 weeks (e.g., by about 20 weeks, 16 weeks, 12 weeks, 8 weeks, or 4 weeks) after administration of the viral vector to the patient. For example, the change from baseline in quantitative analysis of GAA expression in a muscle biopsy persists for at least 48 weeks after administration of the viral vector to the patient. Upon administering the viral vector to the patient, the patient displays pulmonary function improvement by about 24 weeks (e.g., by about 20 weeks, 16 weeks, 12 weeks, 8 weeks, or 4 weeks) after administration of the viral vector to the patient.

### Example 5. Treatment of Pompe Disease in Human Patients that are 20 Years Old or Older by Administration of a Pseudotyped AAV2/8 Vector Including a Nucleic Acid Sequence Encoding an Acid Alpha-Glucosidase Gene Operably Linked to a MCK Promoter and an Anti-Transaminitis Agent

Using the compositions and methods of the disclosure, a patient having LOPD that is greater than about 20 years old may be administered a pseudotyped AAV2/8 vector including a nucleic acid sequence encoding an GAA gene operably linked to a MCK promoter (e.g., the viral vector), for example, in a dose of from about 1×10¹³ vg/kg to about 3×10¹⁴ vg/kg (e.g., from about 1×10¹³ vg/kg to about 6×10¹³ vg/kg, from about 1×10¹³ vg/kg to about 5×10¹³ vg/kg, from about 1×10¹³ vg/kg to about 4×10¹³ vg/kg, from about 1×10¹³ vg/kg to about 3×10¹³ vg/kg, from about 2×10¹³ vg/kg to about 6×10¹³ vg/kg, from about 2×10¹³ vg/kg to about 5×10¹³ vg/kg, or from about 2×10¹³ vg/kg to about 4×10¹³ vg/kg). Afterwards, it is determined that the patient exhibits transaminitis or hyperbilirubinemia or one or more symptoms thereof, and the patient is be administered an anti-transaminitis agent (e.g., prednisolone), for example, in one or doses of about 0.1 mg/kg/dose to about 2 mg/kg/dose and by way of a unit dosage form including 5 mg, 10 mg, 15, mg, or 30 mg and/or from 1 mg/day to about 120 mg/day.

Upon administering the pseudotyped AAV2/8 vector including a nucleic acid sequence encoding an GAA gene operably linked to a MCK promoter (e.g., the viral vector) to the patient, the patient displays a change from baseline in quantitative analysis of GAA expression in a muscle biopsy. For example, the patient displays the change from baseline in quantitative analysis of GAA expression in a muscle biopsy by about 24 weeks (e.g., by about 20 weeks, 16 weeks, 12 weeks, 8 weeks, or 4 weeks) after administration of the pseudotyped AAV2/8 vector including a nucleic acid sequence encoding an GAA gene operably linked to a MCK promoter (e.g., the viral vector) to the patient. For example, the change from baseline in quantitative analysis of GAA expression in a muscle biopsy persists for at least 48 weeks after administration of the viral vector to the patient.

### Example 6. Treatment of Pompe Disease in Human Patients that are One Year Old or Younger by Administration of the Viral Vector and an Anti-Transaminitis Agent

Using the compositions and methods of the disclosure, a patient having Pompe disease that is less than about one year old may be administered the viral vector, for example, in a dose of from about 1×10¹³ vg/kg to about 3×10¹⁴ vg/kg (e.g., from about 1×10¹³ vg/kg to about 6×10¹³ vg/kg, from about 1×10¹³ vg/kg to about 5×10¹³ vg/kg, from about 1×10¹³ vg/kg to about 4×10¹³ vg/kg, from about 1×10¹³ vg/kg to about 3×10¹³ vg/kg, from about 2×10¹³ vg/kg to about 6×10¹³ vg/kg, from about 2×10¹³ vg/kg to about 5×10¹³ vg/kg, or from about 2×10¹³ vg/kg to about 4×10¹³ vg/kg). Afterwards, it is determined that the patient exhibits transaminitis or hyperbilirubinemia or one or more symptoms thereof, and the patient is be administered an anti-transaminitis agent (e.g., prednisolone), for example, in doses of about 0.1 mg/kg/dose to about 2 mg/kg/dose and by way of a unit dosage form including 5 mg, 10 mg, 15, mg, or 30 mg and/or from 1 mg/day to about 120 mg/day.

Upon administering the viral vector to the patient, the patient exhibits a reduction in glycogen in skeletal muscle, cardiac muscle, and/or neuronal tissue. For example, the patient displays the reduction in glycogen in skeletal muscle, cardiac muscle, and/or neuronal tissue by about 24 weeks (e.g., by about 20 weeks, 16 weeks, 12 weeks, 8 weeks, or 4 weeks) after administration of the viral vector to the patient.

### Example 7. Treatment or Prevention of Transaminitis or Hyperbilirubinemia in a Human Patient that has Pompe Disease by Administration of an Anti-Transaminitis Agent

Using the compositions and methods of the disclosure, a patient having Pompe disease that was previously administered the viral vector in a dose of from about 1×10¹³ vg/kg to about 3×10¹⁴ vg/kg (e.g., from about 1×10¹³ vg/kg to about 6×10¹³ vg/kg, from about 1×10¹³ vg/kg to about 5×10¹³ vg/kg, from about 1×10¹³ vg/kg to about 4×10¹³ vg/kg, from about 1×10¹³ vg/kg to about 3×10¹³ vg/kg, from about 2×10¹³ vg/kg to about 6×10¹³ vg/kg, from about 2×10¹³ vg/kg to about 5×10¹³ vg/kg, or from about 2×10¹³ vg/kg to about 4×10¹³ vg/kg), is administered an anti-transaminitis agent (e.g., prednisolone), for example, in doses of about 0.1 mg/kg/dose to about 2 mg/kg/dose and by way of a unit dosage form including 5 mg, 10 mg, 15, mg, or 30 mg and/or from 1 mg/day to about 120 mg/day.

Upon administering the pseudotyped AAV2/8 vector including a nucleic acid sequence encoding an GAA gene operably linked to a MCK promoter (e.g., the viral vector) to the patient, the patient exhibits endogenous GAA activity of about 50% to about 200% of the endogenous GAA activity of a human of the same gender and similar body mass index that does not have Pompe disease by about 24 weeks (e.g., by about 20 weeks, 16 weeks, 12 weeks, 8 weeks, or 4 weeks) after administration of the pseudotyped AAV2/8 vector including a nucleic acid sequence encoding an GAA gene operably linked to a MCK promoter (e.g., the viral vector) to the patient.

### Example 8. Treatment or Prevention of Transaminitis or Hyperbilirubinemia in a Human Patient that has Pompe Disease by Administration of Prednisolone

Using the compositions and methods of the disclosure, a patient having Pompe disease that was previously administered the viral vector in a dose of from about 1×10¹³ vg/kg to about 3×10¹⁴ vg/kg (e.g., from about 1×10¹³ vg/kg to about 6×10¹³ vg/kg, from about 1×10¹³ vg/kg to about 5×10¹³ vg/kg, from about 1×10¹³ vg/kg to about 4×10¹³ vg/kg, from about 1×10¹³ vg/kg to about 3×10¹³ vg/kg, from about 2×10¹³ vg/kg to about 6×10¹³ vg/kg, from about 2×10¹³ vg/kg to about 5×10¹³ vg/kg, or from about 2×10¹³ vg/kg to about 4×10¹³ vg/kg) is administered prednisolone, for example, in doses of about 0.1 mg/kg/dose to about 2 mg/kg/dose and by way of a unit dosage form including 5 mg, 10 mg, 15, mg, or 30 mg and/or from 1 mg/day to about 120 mg/day.

### Example 9. Treatment or Prevention of Transaminitis or Hyperbilirubinemia in a Human that is One Year Old or Younger and Who has Pompe Disease by Administration of an Anti-Transaminitis Agent

Using the compositions and methods of the disclosure, a patient that is one year old or younger having Pompe disease that was previously administered the viral vector, for example, in a dose of from about 1×10¹³ vg/kg to about 3×10¹⁴ vg/kg (e.g., from about 1×10¹³ vg/kg to about 6×10¹³ vg/kg, from about 1×10¹³ vg/kg to about 5×10¹³ vg/kg, from about 1×10¹³ vg/kg to about 4×10¹³ vg/kg, from about 1×10¹³ vg/kg to about 3×10¹³ vg/kg, from about 2×10¹³ vg/kg to about 6×10¹³ vg/kg, from about 2×10¹³ vg/kg to about 5×10¹³ vg/kg, or from about 2×10¹³ vg/kg to about 4×10¹³ vg/kg), is administered an anti-transaminitis agent (e.g., prednisolone). The anti-transaminitis agent is, for example, administered in doses of about 0.1 mg/kg/dose to about 2 mg/kg/dose and by way of a unit dosage form including 5 mg, 10 mg, 15, mg, or 30 mg and/or from 1 mg/day to about 120 mg/day.

Upon administering the pseudotyped AAV2/8 vector including a nucleic acid sequence encoding an GAA gene operably linked to a MCK promoter (e.g., the viral vector) to the patient, the patient exhibits endogenous GAA activity of about 50% to about 200% of the endogenous GAA activity of a human of the same gender and similar body mass index that does not have Pompe disease by about 24 weeks (e.g., by about 20 weeks, 16 weeks, 12 weeks, 8 weeks, or 4 weeks) after administration of the pseudotyped AAV2/8 vector including a nucleic acid sequence encoding an GAA gene operably linked to a MCK promoter (e.g., the viral vector) to the patient.

### Other Embodiments

In addition to the sections outlined above, the compositions and methods of the present disclosure are also captured in the following enumerated embodiments:

[1] A method of treating Pompe disease in a human patient in need thereof, the method comprising administering to the patient (i) a therapeutically effective amount of a transgene encoding GAA and (ii) an anti-transaminitis agent.

[2] A method of reducing glycogen accumulation in muscle tissue in a human patient diagnosed as having Pompe disease, the method comprising administering to the patient (i) a therapeutically effective amount of a transgene encoding GAA and (ii) an anti-transaminitis agent.

[3] A method of improving pulmonary function in a human patient diagnosed as having Pompe disease, the method comprising administering to the patient (i) a therapeutically effective amount of a transgene encoding GAA and (ii) an anti-transaminitis agent.

[4] A method of increasing GAA expression in a human patient diagnosed as having Pompe disease, the method comprising administering to the patient (i) a therapeutically effective amount of a viral vector comprising a transgene encoding GAA and (ii) an anti-transaminitis agent.

[5] The method of any one of embodiments 1-4, wherein the transgene encoding GAA is administered to the patient by transduction with a viral vector comprising a transgene encoding GAA.

[6] The method of any one of embodiments 1-5, wherein the anti-transaminitis agent is administered to the patient in one or more (e.g., one or more, two or more, three or more, four or more, five or more, six or more, seven or more, eight or more, nine or more, or ten or more) doses that commence within 48 weeks (e.g., 48 weeks before or five weeks after) of administration of the transgene or viral vector to the patient.

[7] The method of embodiment 6, wherein the anti-transaminitis agent is administered to the patient in one or more (e.g., one or more, two or more, three or more, four or more, five or more, six or more, seven or more, eight or more, nine or more, or ten or more) doses that commence within 36 weeks (e.g., 36 weeks before or four weeks after) of administration of the transgene or viral vector to the patient.

[8] The method of embodiment 6, wherein the anti-transaminitis agent is administered to the patient in one or more (e.g., one or more, two or more, three or more, four or more, five or more, six or more, seven or more, eight or more, nine or more, or ten or more) doses that commence within 24 weeks (e.g., 24 weeks before or three weeks after) of administration of the transgene or viral vector to the patient.

[9] The method of embodiment 6, wherein the anti-transaminitis agent is administered to the patient in one or more (e.g., one or more, two or more, three or more, four or more, five or more, six or more, seven or more, eight or more, nine or more, or ten or more) doses that commence within 12 weeks (e.g., 12 weeks before or two weeks after) of administration of the transgene or viral vector to the patient.

[10] The method of embodiment 6, wherein the anti-transaminitis agent is administered to the patient in one or more (e.g., one or more, two or more, three or more, four or more, five or more, six or more, seven or more, eight or more, nine or more, or ten or more) doses that commence within one week (e.g., one week before or one week after, six days before or six days after, five days before or five days after, four days before or four days after, three days before or three days after, two days before or two days after, or one day before or one day after) of administration of the transgene or viral vector to the patient.

[11] The method of embodiment 6, wherein the anti-transaminitis agent is administered to the patient in one or more (e.g., one or more, two or more, three or more, four or more, five or more, six or more, seven or more, eight or more, nine or more, or ten or more) doses that commence on the same day (e.g., 24^{th} hour, on the 23^{rd} hour, on the 22^{nd} hour, on the 21^{st} hour, on the 20^{th} hour, on the 19^{th} hour, on the 18^{th} hour, on the 17^{th} hour, on the 16^{th} hour, on the 15^{th} hour, on the 14^{th} hour, on the 13^{th} hour, on the 12^{th} hour, on the 11^{th} hour, on the 10^{th} hour, on the 9^{th} hour, on the 8^{th} hour, on the 7^{th} hour, on the 6^{th} hour, on the 5^{th} hour, on the 4^{th} hour, on the 3^{rd} hour, on the 2^{nd} hour, on the 1^{st} hour, on the 60^{th} minute, on the 59^{th} minute, on the 58^{th} minute, on the 57^{th} minute, on the 56^{th} minute, on the 55^{th} minute, on the 50^{th} minute, on the 40^{th} minute, on the 30^{th} minute, on the 20^{th} minute, on the 10^{th} minute, or on the same minute) as administration of the transgene or viral vector to the patient.

[12] A method of treating Pompe disease in a human patient in need thereof and who has been previously administered an anti-transaminitis agent, the method comprising administering to the patient a therapeutically effective amount of a transgene encoding GAA.

[13] A method of reducing glycogen accumulation in muscle tissue in a human patient diagnosed as having Pompe disease and who has been previously administered an anti-transaminitis agent, the method comprising administering to the patient a therapeutically effective amount of a transgene encoding GAA.

[14] A method of improving pulmonary function in a human patient diagnosed as having Pompe disease and who has been previously administered an anti-transaminitis agent, the method comprising administering to the patient a therapeutically effective amount of a transgene encoding GAA.

[15] A method of increasing GAA expression in a human patient diagnosed as having Pompe disease and who has been previously administered an anti-transaminitis agent, the method comprising administering to the patient a therapeutically effective amount of a transgene encoding GAA.

[16] The method of any one of embodiments 12-15, wherein the transgene encoding GAA was administered to the patient by transduction with a viral vector comprising a transgene encoding GAA.

[17] The method of any one of embodiments 5-11 or 16, wherein the viral vector was administered to the patient in an amount of from 1×10¹³ vg/kg to 3×10¹⁴ vg/kg (e.g., from 1×10¹³ vg/kg to 6×10¹³ vg/kg, from 1×10¹³ vg/kg to 5×10¹³ vg/kg, from 1×10¹³ vg/kg to 4×10¹³ vg/kg, from 1×10¹³ vg/kg to 3×10¹³ vg/kg, from 2×10¹³ vg/kg to 6×10¹³ vg/kg, from 2×10¹³ vg/kg to 5×10¹³ vg/kg, or from 2×10¹³ vg/kg to 4×10¹³ vg/kg, such as in an amount of 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, or 3×10¹⁴ vg/kg).

[18] The method of embodiment 17, wherein the viral vector was administered to the patient in an amount of from 1×10¹⁴ vg/kg to 2×10¹⁴ vg/kg (e.g., in amount of 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg).

[19] The method of any one of embodiments 1-18, wherein the patient was one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older) at the time of administration of the transgene or viral vector.

[20] The method of embodiment 19, wherein the patient was 18 years old or older (e.g., 19 years old or older, 20 years old or older, 25 years old or older, 30 years old or older, 40 years old or older, or 50 years old or older) at the time of administration of the transgene or viral vector.

[21] The method of any one of embodiments 1-20, wherein the patient was from one year old to 40 years old (e.g., 1 year old to 35 years old, 2 years old to 30 years old, 3 years old to 25 years old, 4 years old to 20 years old, or 18 years old) at the time of administration of the transgene or viral vector.

[22] The method of any one of embodiments 1-21, the method further comprising monitoring the patient for development of transaminasemia, hyperbilirubinemia, or one or more symptoms thereof.

[23] The method of embodiment 22, wherein the patient is monitored for the development of transaminasemia, hyperbilirubinemia, or one or more symptoms thereof by evaluating a parameter in a blood sample obtained from the patient, wherein a finding that the parameter is above a reference level identifies the patient as having transaminasemia, hyperbilirubinemia, or one or more symptoms thereof.

[24] The method of embodiment 23, wherein the parameter comprises the level of aspartate aminotransferase or alanine aminotransferase in the blood sample.

[25] A method of treating Pompe disease in a human patient in need thereof, the method comprising:
(a) administering to the patient a transgene encoding GAA,
(b) monitoring the patient for development of transaminasemia, hyperbilirubinemia, or one or more symptoms thereof, and, if the patient exhibits transaminasemia, hyperbilirubinemia, or one or more symptoms thereof,
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[26] A method of reducing glycogen accumulation in muscle tissue in a human patient diagnosed as having Pompe disease, the method comprising:
(a) administering to the patient a transgene encoding GAA,
(b) monitoring the patient for development of transaminasemia, hyperbilirubinemia, or one or more symptoms thereof, and, if the patient exhibits transaminasemia, hyperbilirubinemia, or one or more symptoms thereof,
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[27] A method of improving pulmonary function in a human patient diagnosed as having Pompe disease, the method comprising:
a) administering to the patient a transgene encoding GAA,
(b) monitoring the patient for development of transaminasemia, hyperbilirubinemia, or one or more symptoms thereof, and, if the patient exhibits transaminasemia, hyperbilirubinemia, or one or more symptoms thereof,
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[28] A method of increasing GAA expression in a human patient diagnosed as having Pompe disease, the method comprising:
a) administering to the patient a transgene encoding GAA,
(b) monitoring the patient for development of transaminasemia, hyperbilirubinemia, or one or more symptoms thereof, and, if the patient exhibits transaminasemia, hyperbilirubinemia, or one or more symptoms thereof,
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[29] The method of any one of embodiments 25-28, wherein the transgene encoding GAA is administered to the patient by transduction with a viral vector comprising a transgene encoding GAA.

[30] A method of treating Pompe disease in a human patient in need thereof, the method comprising:
(a) administering to the patient a transgene encoding GAA,
(b) determining that the patient exhibits transaminasemia, hyperbilirubinemia, or one or more symptoms thereof, and
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[31] A method of reducing glycogen accumulation in muscle tissue in a human patient diagnosed as having Pompe disease, the method comprising:
(a) administering to the patient a transgene encoding GAA,
(b) determining that the patient exhibits transaminasemia, hyperbilirubinemia, or one or more symptoms thereof, and
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[32] A method of improving pulmonary function in a human patient diagnosed as having Pompe disease, the method comprising:
(a) administering to the patient a transgene encoding GAA,
(b) determining that the patient exhibits transaminasemia, hyperbilirubinemia, or one or more symptoms thereof, and
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[33] A method of increasing GAA expression in a human patient diagnosed as having Pompe disease, the method comprising:
(a) administering to the patient a transgene encoding GAA,
(b) determining that the patient exhibits transaminasemia, hyperbilirubinemia, or one or more symptoms thereof, and
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[34] The method of any one of embodiments 29-33, wherein the transgene encoding GAA is administered to the patient by transduction with a viral vector comprising a transgene encoding GAA.

[35] The method of embodiment 29-34, wherein the viral vector is administered to the patient in an amount of from 1×10¹³ vg/kg to 3×10¹⁴ vg/kg (e.g., from 1×10¹³ vg/kg to 6×10¹³ vg/kg, from 1×10¹³ vg/kg to 5×10¹³ vg/kg, from 1×10¹³ vg/kg to 4×10¹³ vg/kg, from 1×10¹³ vg/kg to 3×10¹³ vg/kg, from 2×10¹³ vg/kg to 6×10¹³ vg/kg, from 2×10¹³ vg/kg to 5×10¹³ vg/kg, or from 2×10¹³ vg/kg to 4×10¹³ vg/kg, such as in an amount of 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, or 3×10¹⁴ vg/kg).

[36] The method of embodiment 35, wherein the viral vector is administered to the patient in an amount of from 1×10¹⁴ vg/kg to 2×10¹⁴ vg/kg (e.g., in amount of 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg).

[37] The method of embodiment 36, wherein the patient is one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older) at the time of administration of the transgene or viral vector.

[38] The method of embodiment 36, wherein the patient is 18 years old or older (e.g., 19 years old or older, 20 years old or older, 25 years old or older, 30 years old or older, 40 years old or older, or 50 years old or older) at the time of administration of the transgene or viral vector.

[39] The method of any one of embodiments 25-36, wherein the patient is from one year old to 40 years old (e.g., 1 year old to 35 years old, 2 years old to 30 years old, 3 years old to 25 years old, 4 years old to 20 years old, or 18 years old) at the time of administration of the transgene or viral vector.

[40] A method of treating Pompe disease in a human patient in need thereof that is one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older), the method comprising:
(a) administering to the patient a therapeutically effective amount of a transgene encoding GAA,
(b) monitoring the patient for development of transaminasemia, hyperbilirubinemia, or one or more symptoms thereof, and, if the patient exhibits transaminasemia, hyperbilirubinemia, or one or more symptoms thereof,
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[41] A method of reducing glycogen accumulation in muscle tissue in a human patient diagnosed as having Pompe disease, the method comprising:
(a) administering to the patient a therapeutically effective amount of a transgene encoding GAA,
(b) monitoring the patient for development of transaminasemia, hyperbilirubinemia, or one or more symptoms thereof, and, if the patient exhibits transaminasemia, hyperbilirubinemia, or one or more symptoms thereof,
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[42] A method of improving pulmonary function in a human patient diagnosed as having Pompe disease, the method comprising:
(a) administering to the patient a therapeutically effective amount of a transgene encoding GAA,
(b) monitoring the patient for development of transaminasemia, hyperbilirubinemia, or one or more symptoms thereof, and, if the patient exhibits transaminasemia, hyperbilirubinemia, or one or more symptoms thereof,
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[43] A method of increasing GAA expression in a human patient diagnosed as having Pompe disease, the method comprising:
(a) administering to the patient a therapeutically effective amount of a transgene encoding GAA,
(b) monitoring the patient for development of transaminasemia, hyperbilirubinemia, or one or more symptoms thereof, and, if the patient exhibits transaminasemia, hyperbilirubinemia, or one or more symptoms thereof,
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[44] The method of any one of embodiments 40-43, wherein the transgene encoding GAA is administered to the patient by transduction with a viral vector comprising a transgene encoding GAA comprising a transgene encoding GAA.

[45] A method of treating Pompe disease in a human patient in need thereof that is one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older), the method comprising:
(a) administering to the patient a therapeutically effective amount of a transgene encoding GAA,
(b) determining that the patient exhibits transaminasemia, hyperbilirubinemia, or one or more symptoms thereof, and
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[46] A method of reducing glycogen accumulation in muscle tissue in a human patient diagnosed as having Pompe disease that is one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older), the method comprising:
(a) administering to the patient a therapeutically effective amount of a transgene encoding GAA,
(b) determining that the patient exhibits transaminasemia, hyperbilirubinemia, or one or more symptoms thereof, and
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[47] A method of improving pulmonary function in a human patient diagnosed as having Pompe disease that is one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older), the method comprising:
(a) administering to the patient a therapeutically effective amount of a transgene encoding GAA,
(b) determining that the patient exhibits transaminasemia, hyperbilirubinemia, or one or more symptoms thereof, and
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[48] A method of increasing GAA expression in a human patient diagnosed as having Pompe disease that is one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older), the method comprising:
(a) administering to the patient a therapeutically effective amount of a transgene encoding GAA,
(b) determining that the patient exhibits transaminasemia, hyperbilirubinemia, or one or more symptoms thereof, and
(c) (i) administering to the patient an anti-transaminitis agent, (ii) re-administering an anti-transaminitis agent to the patient, wherein the patient had previously been treated with an anti-transaminitis agent upon administration of the viral vector, or (iii) increasing the dosage and/or frequency of an anti-transaminitis agent that is being provided to the patient.

[49] The method of any one of embodiments 40-48, wherein the transgene encoding GAA is administered to the patient by transduction with a viral vector comprising a transgene encoding GAA comprising a transgene encoding GAA.

[50] The method of embodiment 49, wherein the patient is one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older) at the time of administration of the transgene or viral vector.

[51] The method of any one of embodiments 40-50, wherein the patient is from one year old to 40 years old (e.g., 1 year old to 35 years old, 2 years old to 30 years old, 3 years old to 25 years old, 4 years old to 20 years old, or 18 years old) at the time of administration of the transgene or viral vector.

[52] The method of any one of embodiments 44 or 49-51, wherein the viral vector is administered to the patient in an amount of from 1×10¹³ vg/kg to 3×10¹⁴ vg/kg (from 1×10¹³ vg/kg to 6×10¹³ vg/kg, from 1×10¹³ vg/kg to 5×10¹³ vg/kg, from 1×10¹³ vg/kg to 4×10¹³ vg/kg, from 1×10¹³ vg/kg to 3×10¹³ vg/kg, from 2×10¹³ vg/kg to 6×10¹³ vg/kg, from 2×10¹³ vg/kg to 5×10¹³ vg/kg, or from 2×10¹³ vg/kg to 4×10¹³ vg/kg, such as in an amount of 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, or 3×10¹⁴ vg/kg).

[53] The method of embodiment 52, wherein the viral vector is administered to the patient in an amount of from 1×10¹⁴ vg/kg to 2×10¹⁴ vg/kg (e.g., in amount of 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg).

[54] A method of treating or preventing transaminasemia or hyperbilirubinemia in a human patient that has Pompe disease and who has been previously administered a transgene encoding GAA, the method comprising administering to the patient an anti-transaminitis agent.

[55] A method of treating or preventing transaminasemia or hyperbilirubinemia in a human patient that has Pompe disease and who has been previously administered a viral vector comprising a transgene encoding GAA, the method comprising administering to the patient an anti-transaminitis agent.

[56] The method of embodiment 55, wherein the viral vector was administered to the patient in an amount of from 1×10¹³ vg/kg to 6×10¹³ vg/kg, from 1×10¹³ vg/kg to 5×10¹³ vg/kg, from 1×10¹³ vg/kg to 4×10¹³ vg/kg, from 1×10¹³ vg/kg to 3×10¹³ vg/kg, from 2×10¹³ vg/kg to 6×10¹³ vg/kg, from 2×10¹³ vg/kg to 5×10¹³ vg/kg, or from 2×10¹³ vg/kg to 4×10¹³ vg/kg, such as in an amount of 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, or 3×10¹⁴ vg/kg.

[57] The method of embodiment 56, wherein the viral vector was administered to the patient in an amount of from 1×10¹⁴ vg/kg to 2×10¹⁴ vg/kg (e.g., in amount of 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, or 2×10¹⁴ vg/kg).

[58] The method of embodiment 57, wherein the patient was one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older) at the time of administration of the transgene or viral vector.

[59] The method of embodiment 58, wherein the patient was 18 years old or older (e.g., 19 years old or older, 20 years old or older, 25 years old or older, 30 years old or older, 40 years old or older, or 50 years old or older) at the time of administration of the transgene or viral vector.

[60] The method of any one of embodiments 54-59, wherein the patient was from one year old to 40 years old (e.g., 1 year old to 35 years old, 2 years old to 30 years old, 3 years old to 25 years old, 4 years old to 20 years old, or 18 years old) at the time of administration of the transgene or viral vector.

[61] A method of treating or preventing transaminasemia or hyperbilirubinemia in a human patient that has Pompe disease, has been previously administered a transgene encoding GAA, and that was one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older) at the time of administration of the transgene, the method comprising administering to the patient an anti-transaminitis agent.

[62] A method of treating or preventing transaminasemia or hyperbilirubinemia in a human patient that has Pompe disease, has been previously administered a viral vector comprising a transgene encoding GAA, and that was one year old or older (e.g., 2 years old or older, 3 years old or older, 4 years old or older, 5 years old or older, 6 years old or older, 7 years old or older, 8 years old or older, 9 years old or older, 10 years old or older, 15 years old or older, 20 years old or older, 30 years old or older, or 40 month old or older) at the time of administration of the transgene or viral vector.

[63] The method of embodiment 62, wherein the patient was 18 years old or older (e.g., 19 years old or older, 20 years old or older, 25 years old or older, 30 years old or older, 40 years old or older, or 50 years old or older) at the time of administration of the transgene or viral vector.

[64] The method of embodiment 61 or 62, wherein the patient was from one year old to 40 years old (e.g., 1 year old to 35 years old, 2 years old to 30 years old, 3 years old to 25 years old, 4 years old to 20 years old, or 18 years old) at the time of administration of the transgene or viral vector.

[65] The method of any one of embodiments 62-64, wherein the viral vector was administered to the patient in an amount of from 1×10¹³ vg/kg to 3×10¹⁴ vg/kg (from 1×10¹³ vg/kg to 6×10¹³ vg/kg, from 1×10¹³ vg/kg to 5×10¹³ vg/kg, from 1×10¹³ vg/kg to 4×10¹³ vg/kg, from 1×10¹³ vg/kg to 3×10¹³ vg/kg, from 2×10¹³ vg/kg to 6×10¹³ vg/kg, from 2×10¹³ vg/kg to 5×10¹³ vg/kg, or from 2×10¹³ vg/kg to 4×10¹³ vg/kg, such as in an amount of 1×10¹³ vg/kg, 1.1×10¹³ vg/kg, 1.2×10¹³ vg/kg, 1.3×10¹³ vg/kg, 1.4×10¹³ vg/kg, 1.5×10¹³ vg/kg, 1.6×10¹³ vg/kg, 1.7×10¹³ vg/kg, 1.8×10¹³ vg/kg, 1.9×10¹³ vg/kg, 2×10¹³ vg/kg, 2.1×10¹³ vg/kg, 2.2×10¹³ vg/kg, 2.3×10¹³ vg/kg, 2.4×10¹³ vg/kg, 2.5×10¹³ vg/kg, 2.6×10¹³ vg/kg, 2.7×10¹³ vg/kg, 2.8×10¹³ vg/kg, 2.9×10¹³ vg/kg, 3×10¹³ vg/kg, 3.1×10¹³ vg/kg, 3.2×10¹³ vg/kg, 3.3×10¹³ vg/kg, 3.4×10¹³ vg/kg, 3.5×10¹³ vg/kg, 3.6×10¹³ vg/kg, 3.7×10¹³ vg/kg, 3.8×10¹³ vg/kg, 3.9×10¹³ vg/kg, 4×10¹³ vg/kg, 4.1×10¹³ vg/kg, 4.2×10¹³ vg/kg, 4.3×10¹³ vg/kg, 4.4×10¹³ vg/kg, 4.5×10¹³ vg/kg, 4.6×10¹³ vg/kg, 4.7×10¹³ vg/kg, 4.8×10¹³ vg/kg, 4.9×10¹³ vg/kg, 5×10¹³ vg/kg, 5.1×10¹³ vg/kg, 5.2×10¹³ vg/kg, 5.3×10¹³ vg/kg, 5.4×10¹³ vg/kg, 5.5×10¹³ vg/kg, 5.6×10¹³ vg/kg, 5.7×10¹³ vg/kg, 5.8×10¹³ vg/kg, 5.9×10¹³ vg/kg, 6×10¹³ vg/kg, 6.1×10¹³ vg/kg, 6.2×10¹³ vg/kg, 6.3×10¹³ vg/kg, 6.4×10¹³ vg/kg, 6.5×10¹³ vg/kg, 6.6×10¹³ vg/kg, 6.7×10¹³ vg/kg, 6.8×10¹³ vg/kg, 6.9×10¹³ vg/kg, 7×10¹³ vg/kg, 7.1×10¹³ vg/kg, 7.2×10¹³ vg/kg, 7.3×10¹³ vg/kg, 7.4×10¹³ vg/kg, 7.5×10¹³ vg/kg, 7.6×10¹³ vg/kg, 7.7×10¹³ vg/kg, 7.8×10¹³ vg/kg, 7.9×10¹³ vg/kg, 8×10¹³ vg/kg, 8.1×10¹³ vg/kg, 8.2×10¹³ vg/kg, 8.3×10¹³ vg/kg, 8.4×10¹³ vg/kg, 8.5×10¹³ vg/kg, 8.6×10¹³ vg/kg, 8.7×10¹³ vg/kg, 8.8×10¹³ vg/kg, 8.9×10¹³ vg/kg, 9×10¹³ vg/kg, 9.1×10¹³ vg/kg, 9.2×10¹³ vg/kg, 9.3×10¹³ vg/kg, 9.4×10¹³ vg/kg, 9.5×10¹³ vg/kg, 9.6×10¹³ vg/kg, 9.7×10¹³ vg/kg, 9.8×10¹³ vg/kg, 9.9×10¹³ vg/kg, 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10¹⁴ vg/kg, 1.6×10¹⁴ vg/kg, 1.7×10¹⁴ vg/kg, 1.8×10¹⁴ vg/kg, 1.9×10¹⁴ vg/kg, 2×10¹⁴ vg/kg, 2.1×10¹⁴ vg/kg, 2.2×10¹⁴ vg/kg, 2.3×10¹⁴ vg/kg, 2.4×10¹⁴ vg/kg, 2.5×10¹⁴ vg/kg, 2.6×10¹⁴ vg/kg, 2.7×10¹⁴ vg/kg, 2.8×10¹⁴ vg/kg, 2.9×10¹⁴ vg/kg, or 3×10¹⁴ vg/kg).

[66] The method of embodiment 65, wherein the viral vector was administered to the patient in an amount of from 1×10¹⁴ vg/kg to 2×10¹⁴ vg/kg (e.g., in amount of 1×10¹⁴ vg/kg, 1.1×10¹⁴ vg/kg, 1.2×10¹⁴ vg/kg, 1.3×10¹⁴ vg/kg, 1.4×10¹⁴ vg/kg, 1.5×10⁴ vg/kg, 1.6×10¹⁴ vg/kg, 1.7×10¹⁴ vg/kg, 1.8×10¹⁴ vg/kg, 1.9×10¹⁴ vg/kg, or 2×10⁴ vg/kg).

[67] The method of any one of embodiments 1-60 and 64-66, wherein the transgene or viral vector is administered to the patient in a single dose comprising the amount.

[68] The method of any one of embodiments 1-60 and 64-66, wherein the transgene or viral vector is administered to the patient in two or more (e.g., two or more, three or more, four or more, five or more, six or more, seven or more, eight or more, nine or more, or ten or more) doses that, together, comprise the amount.

[69] The method of any one of embodiments 1-60 and 64-66, wherein the transgene or viral vector is administered to the patient in two or more (e.g., two or more, three or more, four or more, five or more, six or more, seven or more, eight or more, nine or more, or ten or more) doses that each, individually, comprise the amount.

[70] The method of embodiment 68-69, wherein the two or more (e.g., two or more, three or more, four or more, five or more, six or more, seven or more, eight or more, nine or more, or ten or more) doses are separated from one another by one year or more (e.g., one year or more, two years or more, three years or more, four years or more, or five years or more).

[71] The method of embodiment 68 and 69, wherein the two or more doses are administered to the patient within 12 months (e.g., within 12 months, within 11 months, within 10 months, within 9 months, within 8 months, within 7 months, within 6 months, within 5 months, within 4 months, within 3 months, within 2 months, or within 1 month) of one another.

[72] The method of any one of embodiments 1-71, wherein the viral vector is selected from the group consisting of adeno-associated virus (AAV), adenovirus, lentivirus, retrovirus, poxvirus, baculovirus, herpes simplex virus, vaccinia virus, and a synthetic virus.

[73] The method of embodiment 72, wherein the viral vector is an AAV.

[74] The method of embodiment 73, wherein the AAV is an AAV1, AAV2, AAV3, AAV4, AAV5, AAV6, AAV7, AAV8, AAV9, AAVrh10, or AAVrh74 serotype.

[75] The method of embodiment 73, wherein the viral vector is a pseudotyped AAV.

[76] The method of embodiment 75, wherein the pseudotyped AAV is AAV2/9. [77] The method of embodiment 76, wherein the pseudotyped AAV is AAV2/8. [78] The method of any one of embodiments 1-77, wherein the transgene encoding GAA is operably linked to a promoter that induces expression of the transgene in a muscle and/or neuronal cell.

[79] The method of embodiment 78, wherein the promoter is a muscle MCK promoter, MCK promoter, chicken beta actin promoter, CMV promoter, myosin light chain-2 promoter, alpha actin promoter, troponin 1 promoter, Na⁺/Ca²⁺ exchanger promoter, dystrophin promoter, alpha7 integrin promoter, brain natriuretic peptide promoter, alpha B-crystallin/small heat shock protein promoter, alpha myosin heavy chain promoter, or atrial natriuretic factor promoter.

[80] The method of any one of embodiments 1-78, wherein the viral vector is or was administered to the patient by way of intravenous, intramuscular, intradermal, or subcutaneous administration.

[81] The method of any one of embodiments 1-80, wherein the anti-transaminitis agent is selected from the group consisting of a corticosteroid, a bile acid, a farnesoid X receptor (FXR) ligand, a fibroblast growth factor 19 (FGF-19) mimetic, a Takeda-G-protein-receptor-5 (TGR5) agonist, a peroxisome proliferator-activated receptor (PPAR) agonist, a PPAR-alpha agonist, a PPAR-delta agonist, a dual PPAR-alpha and PPAR-delta agonist, an apical sodium-dependent corticosteroid transporter (ASBT) inhibitor, an immunomodulatory drug, an antifibrotic therapy, and a nicotinamide adenine dinucleotide phosphate oxidase (NOX) inhibitor.

[82] The method of embodiment 81, wherein the corticosteroid is cortisone.

[83] The method of embodiment 81, wherein the corticosteroid is prednisone.

[84] The method of embodiment 81, wherein the corticosteroid is prednisolone.

[85] The method of embodiment 81, wherein the corticosteroid is methylprednisolone.

[86] The method of embodiment 81, wherein the corticosteroid is dexamethasone.

[87] The method of embodiment 81, wherein the corticosteroid is betamethasone.

[88] The method of embodiment 81, wherein the corticosteroid is hydrocortisone.

[89] The method of embodiment 81, wherein the bile acid is ursodeoxycholic acid.

[90] The method of embodiment 81, wherein the bile acid is nor-ursodeoxycholic acid.

[91] The method of embodiment 81, wherein the FXR ligand is obeticholic acid.

[92] The method of embodiment 81, wherein the FXR ligand is cilofexor.

[93] The method of embodiment 81, wherein the FXR ligand is tropifextor.

[94] The method of embodiment 81, wherein the FXR ligand is tretinoin.

[95] The method of embodiment 81, wherein the FXR ligand is EDP-305.

[96] The method of embodiment 81, wherein the FGF-19 mimetic is aldafermin.

[97] The method of embodiment 81, wherein the TGR5 agonist is INT-777.

[98] The method of embodiment 81, wherein the TGR5 agonist is INT-767.

[99] The method of embodiment 81, wherein the PPAR agonist is bezafibrate.

[100] The method of embodiment 81, wherein the PPAR agonist is seladelpar.

[101] The method of embodiment 81, wherein the PPAR agonist is elafibrinor.

[102] The method of embodiment 81, wherein the PPAR-alpha agonist is fenofibrate.

[103] The method of embodiment 81, wherein the PPAR-delta agonist is seladelpar.

[104] The method of embodiment 81, wherein the dual PPAR-alpha and PPAR-delta agonist is elafibranor.

[105] The method of embodiment 81, wherein the ASBT inhibitor is odevixibat.

[106] The method of embodiment 81, wherein the ASBT inhibitor is maralixibat.

[107] The method of embodiment 81, wherein the ASBT inhibitor is linerixibat.

[108] The method of embodiment 81, wherein the immunomodulatory drug is rituximab.

[109] The method of embodiment 81, wherein the immunomodulatory drug is abatacept.

[110] The method of embodiment 81, wherein the immunomodulatory drug is ustekinumab.

[111] The method of embodiment 81, wherein the immunomodulatory drug is infliximab.

[112] The method of embodiment 81, wherein the immunomodulatory drug is baricitinib.

[113] The method of embodiment 81, wherein the immunomodulatory drug is FFP-104.

[114] The method of embodiment 81, wherein the antifibrotic therapy is a vitamin D receptor agonist.

[115] The method of embodiment 81, wherein the antifibrotic therapy is simtuzumab.

[116] The method of embodiment 81, wherein the NOX inhibitor is setanaxib.

[117] The method of embodiment 81, wherein the corticosteroid is prednisolone.

[118] The method of embodiment 81-88 or 117, wherein the corticosteroid is administered to the patient in a single dose.

[119] The method of embodiment 81-88 or 117, wherein the corticosteroid is administered to the patient in a plurality of doses.

[120] The method of embodiment 118 or 119, wherein the corticosteroid is administered to the patient in an amount of from 0.1 mg/kg/dose to 2 mg/kg/dose (e.g., 0.2 mg/kg/dose to 1.9 mg/kg/dose, 0.3 mg/kg/dose to 1.8 mg/kg/dose, 0.4 mg/kg/dose to 1.7 mg/kg/dose, 0.5 mg/kg/dose to 1.6 mg/kg/dose, 1 mg/kg/dose to 1.5 mg/kg/dose.

[121] The method of embodiment 120, wherein the corticosteroid is administered to the patient in an amount of from 0.5 mg/kg/dose, 1 mg/kg/dose, or 2 mg/kg/dose.

[122] The method of embodiment 118 or 119, wherein the corticosteroid is administered to the patient in an amount of from 1 mg to 120 mg (e.g. 2 mg to 119 mg, 3 mg to 118 mg, 4 mg to 117 mg, 5 mg to 116 mg, 10 mg to 115 mg, 20 mg to 110 mg, 30 mg to 100 mg, 40 mg to 90 mg, 50 mg to 80 mg, or 60 mg to 70 mg).

[123] The method of embodiment 122, wherein the corticosteroid is administered to the patient in an amount of 30 mg.

[124] The method of embodiment 122, wherein the corticosteroid is administered to the patient in an amount of 60 mg.

[125] The method of embodiment 122, wherein the corticosteroid is administered to the patient in an amount of 120 mg.

[126] The method of embodiment 81-88 and 117-125, wherein the corticosteroid is administered to the patient in one or more (e.g., one or more, two or more, three or more, four or more, five or more, six or more, seven or more, eight or more, nine or more, or ten or more) doses per day, week, or month.

[127] The method of embodiment 126, wherein the corticosteroid is administered to the patient in one or more (e.g., one or more, two or more, three or more, four or more, five or more, six or more, seven or more, eight or more, nine or more, or ten or more) doses per day.

[128] The method of embodiment 127, wherein the corticosteroid is administered to the patient in one dose per day.

[129] The method of embodiment 127, wherein the corticosteroid is administered to the patient in two doses per day.

[130] The method of embodiment 127, wherein the corticosteroid is administered to the patient in three doses per day.

[131] The method of embodiment 127, wherein the corticosteroid is administered to the patient in four doses per day.

[132] The method of embodiment 127, wherein the corticosteroid is administered to the patient in five doses per day.

[133] The method of any one of embodiments 118-132, wherein the corticosteroid is administered to the patient in an amount of from 1 mg/day to 120 mg/day (e.g., 2 mg/day to 119 mg/day, 3 mg/day to 118 mg/day, 4 mg/day to 117 mg/day, 5 mg/day to 116 mg/day, 10 mg/day to 115 mg/day, 20 mg/day to 110 mg/day, 30 mg/day to 100 mg/day, 40 mg/day to 90 mg/day, 50 mg/day to 80 mg/day, or 60 mg/day to 70 mg/day).

[134] The method of embodiment 133, wherein the corticosteroid is administered to the patient in an amount of from 30 mg/day to 60 mg/day (e.g., 30 mg/day, 30.1 mg/day, 30.2 mg/day, 30.3 mg/day, 30.4 mg/day, 30.5 mg/day, 30.6 mg/day, 30.7 mg/day, 30.8 mg/day, 30.9 mg/day, 31 mg/day, 32 mg/day, 233 mg/day, 34 mg/day, 35 mg/day, 36 mg/day, 37 mg/day, 38 mg/day, 39 mg/day, 40 mg/day, 41 mg/day, 42 mg/day, 43 mg/day, 44 mg/day, 45 mg/day, 46 mg/day, 47 mg/day, 48 mg/day, 49 mg/day, 50 mg/day, 51 mg/day, 52 mg/day, 53 mg/day, 54 mg/day, 55 mg/day, 56 mg/day, 57 mg/day, 58 mg/day, 59 mg/day, or 60 mg/day).

[135] The method of any one of embodiments 81-134, wherein the corticosteroid is administered to the patient in an amount of 30 mg/day.

[136] The method of embodiment 122, wherein the corticosteroid is administered to the patient in an amount of 60 mg/day.

[137] The method of embodiment 122, wherein the corticosteroid is administered to the patient in an amount of 120 mg/day.

[138] The method of any one of embodiments 118-137, wherein the corticosteroid is administered to the patient by way of a unit dosage form comprising 5 mg of the corticosteroid.

[139] The method of any one of embodiments 118-137, wherein the corticosteroid is administered to the patient by way of a unit dosage form comprising 10 mg of the corticosteroid.

[140] The method of any one of embodiments 118-137, wherein the corticosteroid is administered to the patient by way of a unit dosage form comprising 15 mg of the corticosteroid.

[141] The method of any one of embodiments 118-137, wherein the corticosteroid is administered to the patient by way of a unit dosage form comprising 30 mg of the corticosteroid.

[142] The method of any one of embodiments 118-141, wherein the corticosteroid is administered to the patient by way of oral administration.

[143] The method of any one of embodiments 1-142, wherein the patient does not have a history of transaminasemia or hyperbilirubinemia.

[144] The method of embodiment 143, wherein the patient does not have a history of any underlying liver disease.

[145] The method of any one of embodiments 1-144, wherein the patient exhibits a symptom selected from feeding difficulties, failure to thrive, hypotonia, progressive weakness, respiratory distress, severe enlargement of the tongue, and thickening of the heart muscle.

[146] The method of any one of embodiments 1-145, wherein the patient is undergoing GAA enzyme replacement therapy.

[147] The method of any one of embodiments 1-146, wherein upon administering the viral vector to the patient, the patient exhibits endogenous GAA activity of from 50% to 200% of the endogenous GAA activity of a human of the same gender and similar body mass index that does not have Pompe disease.

[148] The method of embodiment 1-147, wherein upon administering the viral vector to the patient, the patient exhibits a reduction in glycogen in skeletal muscle, cardiac muscle, and/or neuronal tissue.

[149] The method of any one of embodiments 30-148, wherein the patient is determined to exhibit transaminasemia or one or more symptoms thereof by a finding that the patient exhibits one or more parameters in a blood test that is increased relative to a reference level.

[150] The method of embodiment 149, wherein the blood test is a liver function test.

[151] The method of embodiment 149 or 150, wherein the one or more parameters comprises the level of aspartate aminotransferase and/or alanine aminotransferase.

[152] The method of any one of embodiments 30-151, the patient is determined to exhibit transaminasemia or one or more symptoms thereof by a finding that the patient exhibits an alanine transaminase level that is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L) in a liver function test.

[153] The method of any one of embodiments 30-152, the patient is determined to exhibit transaminasemia or one or more symptoms thereof by a finding that the patient exhibits an aspartate aminotransferase level that is greater than 50 U/L (e.g., 55 U/L, 60 U/L, 65 U/L, 70 U/L, 75 U/L, 80 U/L, 85 U/L, 90 U/L, 100 U/L, 110 U/L, 120 U/L, 130 U/L, 140 U/L, 150 U/L, 200 U/L, 300 U/L, 400 U/L, and 500 U/L) in a liver function test.

[154] A kit comprising a transgene encoding GAA and a package insert, wherein the package insert instructs a user of the kit to administer the viral vector to a patient having Pompe disease in accordance with the method of any one of embodiments 1-53 or 67-153.

[155] A kit comprising a viral vector comprising a transgene encoding GAA and a package insert, wherein the package insert instructs a user of the kit to administer the anti-transaminitis agent to a patient to treat or prevent transaminasemia or hyperbilirubinemia in accordance with the method of any one of embodiments 1-11 or 25-153.

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating Pompe disease in a human patient in need thereof, the method comprising administering to the patient (i) a therapeutically effective amount of a viral vector comprising a transgene encoding acid alpha-glucosidase (GAA) and (ii) a corticosteroid.

2. A method of reducing glycogen accumulation in muscle tissue and/or in neuronal tissue in a human patient diagnosed as having Pompe disease, the method comprising administering to the patient (i) a therapeutically effective amount of a viral vector comprising a transgene encoding GAA and (ii) a corticosteroid.

3. A method of improving pulmonary function in a human patient diagnosed as having Pompe disease, the method comprising administering to the patient (i) a therapeutically effective amount of a viral vector comprising a transgene encoding GAA and (ii) a corticosteroid.

4. A method of increasing GAA expression in a human patient diagnosed as having Pompe disease, the method comprising administering to the patient (i) a therapeutically effective amount of a viral vector comprising a transgene encoding GAA and (ii) a corticosteroid.

5. The method of any one of claims 1-4, wherein the corticosteroid is administered to the patient in one or more doses that commence within 48 weeks of administration of the viral vector to the patient, optionally wherein the corticosteroid is administered to the patient in one or more doses that commence within 36 weeks or 24 weeks of administration of the viral vector to the patient.

6. The method of any one of claims 1-5, wherein the corticosteroid is administered to the patient in one or more doses that commence within 12 weeks of administration of the viral vector to the patient, optionally wherein the corticosteroid is administered to the patient in one or more doses that commence within 10 weeks, 8 weeks, 6 weeks, or 4 weeks of administration of the viral vector to the patient.

7. The method of claim 6, wherein the corticosteroid is administered to the patient in one or more doses that commence on the same day as administration of the viral vector to the patient.

8. A method of treating Pompe disease in a human patient in need thereof and who has been previously administered a corticosteroid, the method comprising administering to the patient a therapeutically effective amount of a viral vector comprising a transgene encoding GAA.

9. A method of reducing glycogen accumulation in muscle tissue and/or in neuronal tissue in a human patient diagnosed as having Pompe disease and who has been previously administered a corticosteroid, the method comprising administering to the patient a therapeutically effective amount of a viral vector comprising a transgene encoding GAA.

10. A method of improving pulmonary function in a human patient diagnosed as having Pompe disease and who has been previously administered a corticosteroid, the method comprising administering to the patient a therapeutically effective amount of a viral vector comprising a transgene encoding GAA.

11. A method of increasing GAA expression in a human patient diagnosed as having Pompe disease and who has been previously administered a corticosteroid, the method comprising administering to the patient a therapeutically effective amount of a viral vector comprising a transgene encoding GAA.

12. The method of any one of claims 1-11, wherein the viral vector is administered to the patient in an amount of from 1×10¹³ vg/kg to 3×10¹⁴ vg/kg, optionally wherein the viral vector is administered to the patient in an amount of from 1×10¹³ vg/kg to 6×10¹³ vg/kg, from 1×10¹³ vg/kg to 5×10¹³ vg/kg, from 1×10¹³ vg/kg to 4×10¹³ vg/kg, from 1×10¹³ vg/kg to 3×10¹³ vg/kg, from 2×10¹³ vg/kg to 6×10¹³ vg/kg, from 2×10¹³ vg/kg to 5×10¹³ vg/kg, or from 2×10¹³ vg/kg to 4×10¹³ vg/kg.

13. The method of any one of claims 1-12, wherein the viral vector is administered to the patient in an amount of from 3×10¹³ vg/kg to 6×10¹³.

14. The method of any one of claims 1-13, wherein the patient is one year old or older at the time of administration of the viral vector.

15. The method of claim 14, wherein the patient is 18 years old or older at the time of administration of the viral vector.

16. The method of any one of claims 1-13, wherein the patient is from one year old to 40 years old at the time of administration of the viral vector.

17. The method of any one of claims 1-16, the method further comprising monitoring the patient for development of transaminasemia, hyperbilirubinemia, or one or more symptoms thereof.

18. The method of claim 17, wherein the patient is monitored for the development of transaminasemia, hyperbilirubinemia, or one or more symptoms thereof by evaluating a parameter in a blood sample obtained from the patient, wherein a finding that the parameter is above a reference level identifies the patient as having transaminasemia, hyperbilirubinemia, or one or more symptoms thereof.

19. The method of claim 18, wherein the parameter comprises the level of aspartate aminotransferase, alanine aminotransferase, and/or bilirubin in the blood sample.

20. A method of treating Pompe disease in a human patient in need thereof, the method comprising:
(a) administering to the patient a viral vector comprising a transgene encoding GAA in an amount of from 1×10¹³ vg/kg to 3×10¹⁴ vg/kg,
(b) monitoring the patient for development of transaminasemia, hyperbilirubinemia, or one or more symptoms thereof, and, if the patient exhibits transaminasemia, hyperbilirubinemia, or one or more symptoms thereof,
(c) (i) administering to the patient a corticosteroid, (ii) re-administering a corticosteroid to the patient, wherein the patient had previously been treated with a corticosteroid upon administration of the viral vector, or (iii) increasing the dosage and/or frequency of a corticosteroid that is being provided to the patient.

21. A method of reducing glycogen accumulation in muscle tissue and/or in neuronal tissue in a human patient diagnosed as having Pompe disease, the method comprising:
(a) administering to the patient a viral vector comprising a transgene encoding GAA in an amount of from 1×10¹³ vg/kg to 3×10¹⁴ vg/kg,
(b) monitoring the patient for development of transaminasemia, hyperbilirubinemia, or one or more symptoms thereof, and, if the patient exhibits transaminasemia, hyperbilirubinemia, or one or more symptoms thereof,
(c) (i) administering to the patient a corticosteroid, (ii) re-administering a corticosteroid to the patient, wherein the patient had previously been treated with a corticosteroid upon administration of the viral vector, or (iii) increasing the dosage and/or frequency of a corticosteroid that is being provided to the patient.

22. A method of improving pulmonary function in a human patient diagnosed as having Pompe disease, the method comprising:
(a) administering to the patient a viral vector comprising a transgene encoding GAA in an amount of from 1×10¹³ vg/kg to 3×10¹⁴ vg/kg,
(b) monitoring the patient for development of transaminasemia, hyperbilirubinemia, or one or more symptoms thereof, and, if the patient exhibits transaminasemia, hyperbilirubinemia, or one or more symptoms thereof,
(c) (i) administering to the patient a corticosteroid, (ii) re-administering a corticosteroid to the patient, wherein the patient had previously been treated with a corticosteroid upon administration of the viral vector, or (iii) increasing the dosage and/or frequency of a corticosteroid that is being provided to the patient.

23. A method of increasing GAA expression in a human patient diagnosed as having Pompe disease, the method comprising:
(a) administering to the patient a viral vector comprising a transgene encoding GAA in an amount of from 1×10¹³ vg/kg to 3×10¹⁴ vg/kg,
(b) monitoring the patient for development of transaminasemia, hyperbilirubinemia, or one or more symptoms thereof, and, if the patient exhibits transaminasemia, hyperbilirubinemia, or one or more symptoms thereof,
(c) (i) administering to the patient a corticosteroid, (ii) re-administering a corticosteroid to the patient, wherein the patient had previously been treated with a corticosteroid upon administration of the viral vector, or (iii) increasing the dosage and/or frequency of a corticosteroid that is being provided to the patient.

24. A method of treating Pompe disease in a human patient in need thereof, the method comprising:
(a) administering to the patient a viral vector comprising a transgene encoding GAA in an amount of from 1×10¹³ vg/kg to 3×10¹⁴ vg/kg,
(b) determining that the patient exhibits transaminasemia, hyperbilirubinemia, or one or more symptoms thereof, and
(c) (i) administering to the patient a corticosteroid, (ii) re-administering a corticosteroid to the patient, wherein the patient had previously been treated with a corticosteroid upon administration of the viral vector, or (iii) increasing the dosage and/or frequency of a corticosteroid that is being provided to the patient.

25. A method of reducing glycogen accumulation in muscle tissue and/or in neuronal tissue in a human patient diagnosed as having Pompe disease, the method comprising:
(a) administering to the patient a viral vector comprising a transgene encoding GAA in an amount of from 1×10¹³ vg/kg to 3×10¹⁴ vg/kg,
(b) determining that the patient exhibits transaminasemia, hyperbilirubinemia, or one or more symptoms thereof, and
(c) (i) administering to the patient a corticosteroid, (ii) re-administering a corticosteroid to the patient, wherein the patient had previously been treated with a corticosteroid upon administration of the viral vector, or (iii) increasing the dosage and/or frequency of a corticosteroid that is being provided to the patient.

26. A method of improving pulmonary function in a human patient diagnosed as having Pompe disease, the method comprising:
(a) administering to the patient a viral vector comprising a transgene encoding GAA in an amount of from 1×10¹³ vg/kg to 3×10¹⁴ vg/kg,
(b) determining that the patient exhibits transaminasemia, hyperbilirubinemia, or one or more symptoms thereof, and
(c) (i) administering to the patient a corticosteroid, (ii) re-administering a corticosteroid to the patient, wherein the patient had previously been treated with a corticosteroid upon administration of the viral vector, or (iii) increasing the dosage and/or frequency of a corticosteroid that is being provided to the patient.

27. A method of increasing GAA expression in a human patient diagnosed as having Pompe disease, the method comprising:
(a) administering to the patient a viral vector comprising a transgene encoding GAA in an amount of from 1×10¹³ vg/kg to 3×10¹⁴ vg/kg,
(b) determining that the patient exhibits transaminasemia, hyperbilirubinemia, or one or more symptoms thereof, and
(c) (i) administering to the patient a corticosteroid, (ii) re-administering a corticosteroid to the patient, wherein the patient had previously been treated with a corticosteroid upon administration of the viral vector, or (iii) increasing the dosage and/or frequency of a corticosteroid that is being provided to the patient.

28. The method of any one of claims 19-26, wherein the viral vector is administered to the patient in an amount of from 1×10¹³ vg/kg to 6×10¹³ vg/kg, from 1×10¹³ vg/kg to 5×10¹³ vg/kg, from 1×10¹³ vg/kg to 4×10¹³ vg/kg, from 1×10¹³ vg/kg to 3×10¹³ vg/kg, from 2×10¹³ vg/kg to 6×10¹³ vg/kg, from 2×10¹³ vg/kg to 5×10¹³ vg/kg, or from 2×10¹³ vg/kg to 4×10¹³ vg/kg.

29. The method of any one of claims 20-28, wherein the viral vector is administered to the patient in an amount of from 3×10¹³ vg/kg to 6×10¹³.

30. The method of any one of claims 20-29, wherein the patient is one year old or older at the time of administration of the viral vector.

31. The method of claim 30, wherein the patient is 18 years old or older at the time of administration of the viral vector.

32. The method of any one of claims 20-31, wherein the patient is from one year old to 40 years old at the time of administration of the viral vector.

33. A method of treating or preventing transaminasemia or hyperbilirubinemia in a human patient that has Pompe disease and who has been previously administered a viral vector comprising a transgene encoding GAA in an amount of from 1×10¹³ vg/kg to 3×10¹⁴ vg/kg, the method comprising administering to the patient a corticosteroid.

34. The method of claim 33, wherein the viral vector is administered to the patient in an amount of from 1×10¹³ vg/kg to 3×10¹⁴ vg/kg, optionally wherein the viral vector is administered to the patient in an amount of from 2×10¹³ vg/kg to 7×10¹³ vg/kg, from 2×10¹³ vg/kg to 4×10¹³ vg/kg, or from 5×10¹³ vg/kg to 7×10¹³ vg/kg.

35. The method of claim 33 or 34, wherein the viral vector is administered to the patient in an amount of from 3×10¹³ vg/kg to 6×10¹³.

36. The method of any one of claims 33-35, wherein the patient is one year old or older at the time of administration of the viral vector.

37. The method of claim 36, wherein the patient is 18 years old or older at the time of administration of the viral vector.

38. The method of any one of claims 33-36, wherein the patient is from one year old to 40 years old at the time of administration of the viral vector.

39. The method of any one of claims 1-38, wherein the viral vector is administered to the patient in a single dose comprising the amount.

40. The method of any one of claims 1-38, wherein the viral vector is administered to the patient in two or more doses that, together, comprise the amount.

41. The method of any one of claims 1-38, wherein the viral vector is administered to the patient in two or more doses that each, individually, comprise the amount.

42. The method of claim 40 or 41, wherein the two or more doses are separated from one another by one year or more.

43. The method of claim 40 or 41, wherein the two or more doses are administered to the patient within 12 months of one another.

44. The method of any one of claims 1-43, wherein the viral vector is selected from the group consisting of adeno-associated virus (AAV), adenovirus, lentivirus, retrovirus, poxvirus, baculovirus, herpes simplex virus, vaccinia virus, and a synthetic virus.

45. The method of claim 44, wherein the viral vector is an AAV.

46. The method of claim 45, wherein the AAV is an AAV1, AAV2, AAV3, AAV4, AAV5, AAV6, AAV7, AAV8, AAV9, AAVrh10, or AAVrh74 serotype.

47. The method of claim 46, wherein the viral vector is a pseudotyped AAV.

48. The method of claim 47, wherein the pseudotyped AAV is AAV2/8.

49. The method of claim 48, wherein the pseudotyped AAV is AAV2/9.

50. The method of any one of claims 1-49, wherein the transgene encoding GAA is operably linked to a promoter that induces expression of the transgene in a muscle and/or neuronal cell.

51. The method of claim 50, wherein the promoter is a muscle MCK promoter, MCK promoter, chicken beta actin promoter, CMV promoter, myosin light chain-2 promoter, alpha actin promoter, troponin 1 promoter, Na⁺/Ca²⁺ exchanger promoter, dystrophin promoter, alpha7 integrin promoter, brain natriuretic peptide promoter, alpha B-crystallin/small heat shock protein promoter, alpha myosin heavy chain promoter, or atrial natriuretic factor promoter.

52. The method of any one of claims 1-51, wherein the GAA is operably linked to an enhancer that induces expression of the transgene in a muscle and/or neuronal cell.

53. The method of claim 52, wherein the enhancer is a CMV enhancer, a MEF2 enhancer, or a MyoD enhancer.

54. The method of any one of claims 1-53, wherein the viral vector is administered to the patient by way of intravenous, intrathecal, intracisternal, intracerebroventricular, or intramuscular administration to the patient administration.

55. The method of claims 1-54, wherein the corticosteroid is prednisolone.

56. The method of claim 55, wherein the corticosteroid is administered to the patient in a single dose.

57. The method of claim 55 or 56, wherein the corticosteroid is administered to the patient in a plurality of doses.

58. The method of claim 56 or 57, wherein the corticosteroid is administered to the patient in an amount of from 0.1 mg/kg/dose to 2 mg/kg/dose.

59. The method of claim 58, wherein the corticosteroid is administered to the patient in an amount of 0.5 mg/kg/dose, optionally wherein the corticosteroid is administered to the patient in an amount of 1 mg/kg/dose or 2 mg/kg/dose.

60. The method of claim 56 or 57, wherein the corticosteroid is administered to the patient in an amount of from 1 mg to 120 mg.

61. The method of claim 60, wherein the corticosteroid is administered to the patient in an amount of 30 mg.

62. The method of claim 60, wherein the corticosteroid is administered to the patient in an amount of 60 mg.

63. The method of claim 60, wherein the corticosteroid is administered to the patient in an amount of 120 mg.

64. The method of any one of claims 59-63, wherein the corticosteroid is administered to the patient in one or more doses per day, week, or month.

65. The method of claim 64, wherein the corticosteroid is administered to the patient in one or more doses per day, optionally wherein the corticosteroid is administered to the patient in one dose per day, in two doses per day, three doses per day, four doses per day, or five doses per day.

66. The method of claim 65, wherein the corticosteroid is administered to the patient in one dose per day.

67. The method of any one of claims 55-66, wherein the corticosteroid is administered to the patient in an amount of from 1 mg/day to 120 mg/day.

68. The method of any one of claims 55-67, wherein the corticosteroid is administered to the patient in an amount of 30 mg/day.

69. The method of any one of claims 55-67, wherein the corticosteroid is administered to the patient in an amount of 60 mg/day.

70. The method of any one of claims 55-67, wherein the corticosteroid is administered to the patient in an amount of 120 mg/day.

71. The method of any one of claims 55-70, wherein the corticosteroid is administered to the patient by way of a unit dosage form comprising 5 mg of the corticosteroid.

72. The method of any one of claims 55-70, wherein the corticosteroid is administered to the patient by way of a unit dosage form comprising 10 mg of the corticosteroid.

73. The method of any one of claims 55-70, wherein the corticosteroid is administered to the patient by way of a unit dosage form comprising 15 mg of the corticosteroid.

74. The method of any one of claims 55-70, wherein the corticosteroid is administered to the patient by way of a unit dosage form comprising 30 mg of the corticosteroid.

75. The method of any one of claims 55-74, wherein the corticosteroid is administered to the patient by way of oral administration.

76. The method of any one of claims 1-75, wherein the patient does not have a history of transaminasemia or hyperbilirubinemia.

77. The method of claim 76, wherein the patient does not have a history of any underlying liver disease.

78. The method of any one of claims 1-77, wherein the patient is from one year old to 40 years old at the time of administration of the viral vector.

79. The method of any one of claims 1-78, wherein the patient exhibits a symptom selected from feeding difficulties, failure to thrive, hypotonia, progressive weakness, respiratory distress, severe enlargement of the tongue, and thickening of the heart muscle.

80. The method of any one of claims 1-79, wherein the patient is undergoing GAA enzyme replacement therapy.

81. The method of any one of claims 1-80, wherein upon administering the viral vector to the patient, the patient exhibits endogenous GAA activity of from 50% to 200% of the endogenous GAA activity of a human of the same gender and similar body mass index that does not have Pompe disease.

82. The method of any one of claims 1-81, wherein upon administering the viral vector to the patient, the patient exhibits a reduction in glycogen in skeletal muscle, cardiac muscle, and/or neuronal tissue.

83. The method of any one of claims 17-82, wherein the patient is determined to exhibit transaminasemia or one or more symptoms thereof by a finding that the patient exhibits one or more transaminases in a liver function test that is increased relative to a reference level.

84. The method of claim 83, wherein the one or more transaminases comprises the level of aspartate aminotransferase and/or alanine aminotransferase.

85. The method of any one of claims 17-84, wherein the patient is determined to exhibit transaminasemia or one or more symptoms thereof by a finding that the patient exhibits an alanine transaminase level is greater than 50 U/L in a liver function test.

86. The method of any one of claims 17-85, wherein the patient is determined to exhibit transaminasemia or one or more symptoms thereof by a finding that the patient exhibits an aspartate aminotransferase level is greater than 50 U/L in a liver function test.

87. A kit comprising a viral vector comprising a transgene encoding GAA and a package insert, wherein the package insert instructs a user of the kit to administer the viral vector to a patient having Pompe disease in accordance with the method of any one of claims 1-86.

88. A kit comprising a corticosteroid and a package insert, wherein the package insert instructs a user of the kit to administer the corticosteroid to a patient to treat or prevent transaminasemia or hyperbilirubinemia in accordance with the method of any one of claims 1-86.
